SEQUENCE LISTING (21 sequences)

SEQ ID NO: 1 - DNA, 2166 bp, Homo sapiens
  CDS (327)..(1628)
gcacagtggcccttaagcgaggagcggcggcgcccgcagcaatcacagcagtgccgacgtcgtgggtgtttggtgtgaggctgcgagccgccgcgagttctcacggtcccgccggcgccaccaccgcggtcactcaccgccgccgccgccaccactgccaccacggtcgcctgccacagggtcttactctgttgcccaggctggagtgcagtggcacaatcttggctcactgcaacctctgcctcccgggttcaagcaattctcctgcctcagcctcccgagtagctgggattacaggtgtctgcaattgaactccaaggtgcagaatggtttggaaagtagctgtattcctcagtgtggccctgggcattggtgccgttcctatagatgatcctgaagatggaggcaagcactgggtggtgatcgtggcaggttcaaatggctggtataattataggcaccaggcagacgcgtgccatgcctaccagatcattcaccgcaatgggattcctgacgaacagatcgttgtgatgatgtacgatgacattgcttactctgaagacaatcccactccaggaattgtgatcaacaggcccaatggcacagatgtctatcagggagtcccgaaggactacactggagaggatgttaccccacaaaatttccttgctgtgttgagaggcgatgcagaagcagtgaagggcataggatccggcaaagtcctgaagagtggcccccaggatcacgtgttcatttacttcactgaccatggatctactggaatactggtttttcccaatgaagatcttcatgtaaaggacctgaatgagaccatccattacatgtacaaacacaaaatgtaccgaaagatggtgttctacattgaagcctgtgagtctgggtccatgatgaaccacctgccggataacatcaatgtttatgcaactactgctgccaaccccagagagtcgtcctacgcctgttactatgatgagaagaggtccacgtacctgggggactggtacagcgtcaactggatggaagattcggacgtggaagatctgactaaagagaccctgcacaagcagtaccacctggtaaaatcgcacaccaacaccagccacgtcatgcagtatggaaacaaaacaatctccaccatgaaagtgatgcagtttcagggtatgaaacgcaaagccagttctcccgtccccctacctccagtcacacaccttgacctcacccccagccctgatgtgcctctcaccatcatgaaaaggaaactgatgaacaccaatgatctggaggagtccaggcagctcacggaggagatccagcggcatctggatgccaggcacctcattgagaagtcagtgcgtaagatcgtctccttgctggcagcgtccgaggctgaggtggagcagctcctgtccgagagagccccgctcacggggcacagctgctacccagaggccctgctgcacttccggacccactgcttcaactggcactcccccacgtacgagtatgcgttgagacatttgtacgtgctggtcaacctttgtgagaagccgtatccgcttcacaggataaaattgtccatggaccacgtgtgccttggtcactactgaagagctgcctcctggaagcttttccaagtgtgagcgccccaccgactgtgtgctgatcagagactggagaggtggagtgagaagtctccgctgctcgggccctcctggggagcccccgctccagggctcgctccaggaccttcttcacaagatgacttgctcgctgttacctgcttccccagtcttttctgaaaaactacaaattagggtgggaaaagctctgtattgagaagggtcatatttgctttctaggaggtttgttgttttgcctgttagttttgaggagcaggaagctcatgggggcttctgtagcccctctcaaaaggagtctttattctgagaatttgaagctgaaacctctttaaatcttcagaatgattttattgaagagggccgcaagccccaaatggaaaactgtttttagaaaatatgatgatttttgattgcttttgtatttaattctgcaggtgttcaagtcttaaaaaataaagatttataacagaacccaaaaaaaaaaaaaaaaaaaaaaaaaaaaaaa

SEQ ID NO: 2 - protein, 433 aa, Homo sapiens
MVWKVAVFLSVALGIGAVPIDDPEDGGKHWVVIVAGSNGWYNYRHQADACHAYQIIHRNGIPDEQIVVMMYDDIAYSEDNPTPGIVINRPNGTDVYQGVPKDYTGEDVTPQNFLAVLRGDAEAVKGIGSGKVLKSGPQDHVFIYFTDHGSTGILVFPNEDLHVKDLNETIHYMYKHKMYRKMVFYIEACESGSMMNHLPDNINVYATTAANPRESSYACYYDEKRSTYLGDWYSVNWMEDSDVEDLTKETLHKQYHLVKSHTNTSHVMQYGNKTISTMKVMQFQGMKRKASSPVPLPPVTHLDLTPSPDVPLTIMKRKLMNTNDLEESRQLTEEIQRHLDARHLIEKSVRKIVSLLAASEAEVEQLLSERAPLTGHSCYPEALLHFRTHCFNWHSPTYEYALRHLYVLVNLCEKPYPLHRIKLSMDHVCLGHY

SEQ ID NO: 3 - DNA, 2048 bp, Homo sapiens
  CDS (209)..(1510)
gcacagtggcccttaagcgaggagcggcggcgcccgcagcaatcacagcagtgccgacgtcgtgggtgtttggtgtgaggctgcgagccgccgcgagttctcacggtcccgccggcgccaccaccgcggtcactcaccgccgccgccgccaccactgccaccacggtcgcctgccacaggtgtctgcaattgaactccaaggtgcagaatggtttggaaagtagctgtattcctcagtgtggccctgggcattggtgccgttcctatagatgatcctgaagatggaggcaagcactgggtggtgatcgtggcaggttcaaatggctggtataattataggcaccaggcagacgcgtgccatgcctaccagatcattcaccgcaatgggattcctgacgaacagatcgttgtgatgatgtacgatgacattgcttactctgaagacaatcccactccaggaattgtgatcaacaggcccaatggcacagatgtctatcagggagtcccgaaggactacactggagaggatgttaccccacaaaatttccttgctgtgttgagaggcgatgcagaagcagtgaagggcataggatccggcaaagtcctgaagagtggcccccaggatcacgtgttcatttacttcactgaccatggatctactggaatactggtttttcccaatgaagatcttcatgtaaaggacctgaatgagaccatccattacatgtacaaacacaaaatgtaccgaaagatggtgttctacattgaagcctgtgagtctgggtccatgatgaaccacctgccggataacatcaatgtttatgcaactactgctgccaaccccagagagtcgtcctacgcctgttactatgatgagaagaggtccacgtacctgggggactggtacagcgtcaactggatggaagattcggacgtggaagatctgactaaagagaccctgcacaagcagtaccacctggtaaaatcgcacaccaacaccagccacgtcatgcagtatggaaacaaaacaatctccaccatgaaagtgatgcagtttcagggtatgaaacgcaaagccagttctcccgtccccctacctccagtcacacaccttgacctcacccccagccctgatgtgcctctcaccatcatgaaaaggaaactgatgaacaccaatgatctggaggagtccaggcagctcacggaggagatccagcggcatctggatgccaggcacctcattgagaagtcagtgcgtaagatcgtctccttgctggcagcgtccgaggctgaggtggagcagctcctgtccgagagagccccgctcacggggcacagctgctacccagaggccctgctgcacttccggacccactgcttcaactggcactcccccacgtacgagtatgcgttgagacatttgtacgtgctggtcaacctttgtgagaagccgtatccgcttcacaggataaaattgtccatggaccacgtgtgccttggtcactactgaagagctgcctcctggaagcttttccaagtgtgagcgccccaccgactgtgtgctgatcagagactggagaggtggagtgagaagtctccgctgctcgggccctcctggggagcccccgctccagggctcgctccaggaccttcttcacaagatgacttgctcgctgttacctgcttccccagtcttttctgaaaaactacaaattagggtgggaaaagctctgtattgagaagggtcatatttgctttctaggaggtttgttgttttgcctgttagttttgaggagcaggaagctcatgggggcttctgtagcccctctcaaaaggagtctttattctgagaatttgaagctgaaacctctttaaatcttcagaatgattttattgaagagggccgcaagccccaaatggaaaactgtttttagaaaatatgatgatttttgattgcttttgtatttaattctgcaggtgttcaagtcttaaaaaataaagatttataacagaacccaaaaaaaaaaaaaaaaaaaaaaaaaaaaaaa

SEQ ID NO: 4 - protein, 433 aa, Homo sapiens; identical to SEQ ID NO: 2

SEQ ID NO: 5 - DNA, 1889 bp, Mus musculus
  CDS (95)..(1402)
gctctgagtctgcgcgacgcccggaattcccacggttctgcagtcaccgcggcgatcacccgcccagtcttctgtagcggacacggggtgcagaatgacctggagagtggctgtgcttctcagcctggtgctgggtgctggtgccgttcccgtcggtgtggacgatcccgaggatggaggcaagcactgggtggtgattgtggcgggctccaatggctggtataattaccgacaccaggcagacgcatgccacgcctaccagatcatccaccggaacgggattcctgacgagcagatcatagtgatgatgtatgacgacattgccaactctgaagaaaaccctaccccaggtgttgtgatcaaccgacctaacggcacagatgtatacaagggagtcctgaaggactacaccggagaggatgtgactccagagaatttcctcgccgtgctgagaggtgacgcagaagctgtgaagggcaaagggtctggaaaagtcttgaagagtggcccccgagatcatgtcttcatttacttcaccgaccacggagccaccgggatcctggtgtttcctaatgatgatcttcatgtcaaggacctgaataagactattcgctacatgtatgaacacaaaatgtaccagaagatggtgttctacattgaagcttgtgagtctggctccatgatgaaccacctgcccgacgacatcaacgtttatgcaactactgcggccaaccccaaggagtcatcttatgcctgctactacgacgaggagaggggcacttacctgggtgactggtacagcgtcaactggatggaagactccgatgtggaggacctgaccaaagagacccttcacaagcagtaccacctggtcaagtcccacaccaacaccagccatgtcatgcaatatgggaacaaatctatctctaccatgaaagtgatgcagtttcagggaatgaagcacagagccagttcccccatctccctgcctccggtcacacaccttgacctcacccccagccctgacgtgcccctgaccatcttgaagaggaagctgctgagaaccaacgacgtgaaggaatcccagaatctcattgggcagatccagcaatttctggatgccaggcacgtcattgagaagtctgtgcacaagatcgtttccctgctggcgggatttggggaaactgctgagagacatctgtcagagaggaccatgctcacagcacatgactgctaccaggaggctgtaacccacttccgcacacactgctttaactggcactctgtcacgtacgagcatgccttgcggtacttgtatgtgctggccaatctctgtgaggcaccatatccgattgacaggatagagatggccatggacaaagtgtgtcttagtcactactgaacagctcgcttcccaatgagtgagcacagtccactggaatatgaaccaaccagagactgaaagggcggaccagaggcagcacccgcgcccctggcccccaggaatactgcccgcccaccccagggcttgctttttgaagatacctgcttactaagaagccagtttgggtgggtaaagctctctggaagaaggaactttgcttcttaggagtttttttgtttgtttgttttggtttgttttgttgtccattagctttcaagagcaaattccccgcggcttctctagccagggaaggaatcgtctgagaaattcaaagctgaaacctcttgccgtcttcacagtgatttcactgaagaagagggtggaaagcaagcccctatgggagaattatttttagaattatataatttttgattgcttttatattttattctgtaataatggatgttttaaaacaaataagtgaagtgaaaaaaaaaaaaaaaaa

SEQ ID NO: 6 - protein, 435 aa, Mus musculus
MTWRVAVLLSLVLGAGAVPVGVDDPEDGGKHWVVIVAGSNGWYNYRHQADACHAYQIIHRNGIPDEQIIVMMYDDIANSEENPTPGVVINRPNGTDVYKGVLKDYTGEDVTPENFLAVLRGDAEAVKGKGSGKVLKSGPRDHVFIYFTDHGATGILVFPNDDLHVKDLNKTIRYMYEHKMYQKMVFYIEACESGSMMNHLPDDINVYATTAANPKESSYACYYDEERGTYLGDWYSVNWMEDSDVEDLTKETLHKQYHLVKSHTNTSHVMQYGNKSISTMKVMQFQGMKHRASSPISLPPVTHLDLTPSPDVPLTILKRKLLRTNDVKESQNLIGQIQQFLDARHVIEKSVHKIVSLLAGFGETAERHLSERTMLTAHDCYQEAVTHFRTHCFNWHSVTYEHALRYLYVLANLCEAPYPIDRIEMAMDKVCLSHY

SEQ ID NO: 7 - DNA, 1034 bp, Pan troglodytes
  CDS (192)..(1034)
cgaggagcggcggcgcccgcagcaatcacagcagtgcggccgtcgtgggtgtttggtgtgaggctgcgcgccgccgcgagttctcacggtcccgccggcgccaccaccgcggtcactcactgccgccgccgccaccactgccaccacggtcgcctgccacaggtttctgcaattgaactccaaggtgcagaatggtttggaaagtagctgtattcctcagtgtggccctgggcattggtgccgttcctatagatgatcctgaagatggaggcaagcactgggtggtgatcgtggcgggttcaaatggctggtataattataggcaccaggcagacgcgtgccatgcctaccagatcattcaccgcaatgggattcctgacgaacagatcgttgtgatgatgtacgatgacatcgcttactctgaagacaatcccactccaggaattgtgatcaacaggcccaatggcacagatgtctatcagggagtcccgaaggactacaccggagaggatgttaccccacaaaatttccttgctgtgttgagaggcgatgcagaagcagtgaagggcataggatccggcaaagtcctgaagagcggcccccaggatcacgtgttcatttacttcactgaccatggatctactggaatactggtttttcccaatgaagatcttcatgtaaaggacctgaatgagaccatccattacatgtacaaacacaaaatgtaccgaaagatggtgttctacattgaagcctgtgagtctgggtccatgatgaaccacctgccggataacatcaatgtttatgcaactactgctgccaaccccagagagtcgtcctacgcctgttactatgatgagaagaggtccacgtacctgggggactggtacagcgtcaactggatggaagactcggacgtggaagatctgactaaagagaccctgcacaagcagtaccacctggtaaaatcgcacaccaacaccagccacgtcatgcagtatggaaacaaaacaatctccaccatgaaagggtag

SEQ ID NO: 8 - protein, 280 aa, Pan troglodytes
MVWKVAVFLSVALGIGAVPIDDPEDGGKHWVVIVAGSNGWYNYRHQADACHAYQIIHRNGIPDEQIVVMMYDDIAYSEDNPTPGIVINRPNGTDVYQGVPKDYTGEDVTPQNFLAVLRGDAEAVKGIGSGKVLKSGPQDHVFIYFTDHGSTGILVFPNEDLHVKDLNETIHYMYKHKMYRKMVFYIEACESGSMMNHLPDNINVYATTAANPRESSYACYYDEKRSTYLGDWYSVNWMEDSDVEDLTKETLHKQYHLVKSHTNTSHVMQYGNKTISTMKG

SEQ ID NO: 9 - DNA, 1610 bp, Macaca mulatta
  CDS (330)..(1610)
gcgcagtggcccttaagccaggagctgcggcgcccgcagcaatcacagcagtggggccgtcgtgggtggttggtgtgaggctgcgcgccgccgcgagttctcacggtcccgcaggcgccagcagcgcagtcactcaccgccgccgccgccgccaccactgccaccacggtcgcctgccacagggtctcactgtgttacccatgctggagtgcagtggcacaatcttggctcactgcaacctctgcctcctgggttcaagcagttctcctgcctcagcctcccgagtagctgggattacaggtttctgcagttgaactccaaggtgcagaatggtttggaaagtagctgtatttctcagtgtgaccctgggcattggtgctgttcccatagatgatcctgaagatggaggcaagcactgggtagtgatcgtggcgggttcaaatggctggtataattataggcaccaggcagatgcgtgccatgcctaccagatcattcaccggaatgggattcctgacgagcagatcgttgtgatgatgtatgacgacattgcttactctgaagataatcccactccaggaattgtgatcaacaggcccaatggcacggatgtctatcagggagtcccgaaggactacactggagaggatgttaccccacaaaatttccttgctgtgttgagaggcgatgcagaagcagtgaagggcatcggatccgggaaagtcctgaagagcggcccccaggatcacgtgttcgtttacttcactgaccatggatctactggaatactggtttttcccaatgaagatcttcatgtaaaggacctgaatgagaccatccattacatgtacaaacacaaaatgtaccgaaagatggtgttctacattgaagcctgtgagtctgggtccatgatgaaccacctgccggataacatcaatgtttatgcaactactgccgccaaccccagagagtcgtcctacgcctgttactatgatgagaagaggtccacatacctgggggactggtacagcgtcaactggatggaagactcggacgtggaagatctgactaaagagaccctgcacaagcagtaccacctggtgaaatcacacaccaacaccagccacgtcatgcagtacggaaacaaaacgatctccaccatgaaagtgatgcagtttcagggtatgaagcacaaagccagttctcctctctccctgcctccagtcacacacctggacctcacccccagccctgacgtgcccctcacgatcatgaagaggaaactgatgaacaccaatgatctggaggagtccaggcagctcacggaggagatccagcggcatctggatgccaggcacctcattgagaagtcagtgcacaagatcgtctccttgctggcagcgtccgaggctgaggtggagcagctcctgtccgagagagccccgctcacagggcacagctgctacccagaggccctgctgcacttccggacccactgcttcaactggcactcccccacggtgagccggcggggctctccccacgtctggaatccagaagctgaattcatattgtcccccggcagtcacactggatggacaactttatag

SEQ ID NO: 10 - protein, 426 aa, Macaca mulatta
MVWKVAVFLSVTLGIGAVPIDDPEDGGKHWVVIVAGSNGWYNYRHQADACHAYQIIHRNGIPDEQIVVMMYDDIAYSEDNPTPGIVINRPNGTDVYQGVPKDYTGEDVTPQNFLAVLRGDAEAVKGIGSGKVLKSGPQDHVFVYFTDHGSTGILVFPNEDLHVKDLNETIHYMYKHKMYRKMVFYIEACESGSMMNHLPDNINVYATTAANPRESSYACYYDEKRSTYLGDWYSVNWMEDSDVEDLTKETLHKQYHLVKSHTNTSHVMQYGNKTISTMKVMQFQGMKHKASSPLSLPPVTHLDLTPSPDVPLTIMKRKLMNTNDLEESRQLTEEIQRHLDARHLIEKSVHKIVSLLAASEAEVEQLLSERAPLTGHSCYPEALLHFRTHCFNWHSPTVSRRGSPHVWNPEAEFILSPGSHTGWTTL

SEQ ID NO: 11 - DNA, 1131 bp, Homo sapiens
  CDS (1)..(1128)
atggtttggaaagtagctgtattcctcagtgtggccctgggcattggtgccgttcctatagatgatcctgaagatggaggcaagcactgggtggtgatcgtggcaggttcaaatggctggtataattataggcaccaggcagacgcgtgccatgcctaccagatcattcaccgcaatgggattcctgacgaacagatcgttgtgatgatgtacgatgacattgcttactctgaagacaatcccactccaggaattgtgatcaacaggcccaatggcacagatgtctatcagggagtcccgaaggactacactggagaggatgttaccccacaaaatttccttgctgtgttgagaggcgatgcagaagcagtgaagggcataggatccggcaaagtcctgaagagtggcccccaggatcacgtgttcatttacttcactgaccatggatctactggaatactggtttttcccaatgaagatcttcatgtaaaggacctgaatgagaccatccattacatgtacaaacacaaaatgtaccgaaagatggtgttctacattgaagcctgtgagtctgggtccatgatgaaccacctgccggataacatcaatgtttatgcaactactgctgccaaccccagagagtcgtcctacgcctgttactatgatgagaagaggtccacgtacctgggggactggtacagcgtcaactggatggaagactcggacgtggaagatctgactaaagagaccctgcacaagcagtaccacctggtaaaatcgcacaccaacaccagccacgtcatgcagtatggaaacaaaacaatctccaccatgaaagtgatgcagtttcagggtatgaaacgcaaagccagttctcccgtccccctacctccagtcacacaccttgacctcacccccagccctgatgtgcctctcaccatcatgaaaaggaaactgatgaacaccaatgatctggaggagtccaggcagctcacggaggagatccagcggcatctggattacgagtatgcgttgagacatttgtacgtgctggtcaacctttgtgagaagccgtatccgcttcacaggataaaattgtccatggaccacgtgtgccttggtcactactga

SEQ ID NO: 12 - protein, 376 aa, Homo sapiens
MVWKVAVFLSVALGIGAVPIDDPEDGGKHWVVIVAGSNGWYNYRHQADACHAYQIIHRNGIPDEQIVVMMYDDIAYSEDNPTPGIVINRPNGTDVYQGVPKDYTGEDVTPQNFLAVLRGDAEAVKGIGSGKVLKSGPQDHVFIYFTDHGSTGILVFPNEDLHVKDLNETIHYMYKHKMYRKMVFYIEACESGSMMNHLPDNINVYATTAANPRESSYACYYDEKRSTYLGDWYSVNWMEDSDVEDLTKETLHKQYHLVKSHTNTSHVMQYGNKTISTMKVMQFQGMKRKASSPVPLPPVTHLDLTPSPDVPLTIMKRKLMNTNDLEESRQLTEEIQRHLDYEYALRHLYVLVNLCEKPYPLHRIKLSMDHVCLGHY

SEQ ID NO: 13 - protein, 21 aa, Apis mellifera
MKFLVNVALVFMVVYISYIYA

SEQ ID NO: 14 - DNA, 21 bp, Artificial
  Forward primer for quantitative Real-Time PCR
ccaggaggctgtaacccactt

SEQ ID NO: 15 - DNA, 19 bp, Artificial
  Reverse primer for quantitative Real-Time PCR
gcaaggcatgctcgtacgt

SEQ ID NO: 16 - DNA, 49 bp, artificial
  Forward primer for generating murine legumain sense riboprobe for in situ hybridization
gactgataatacgactcactatagggcgaacaccaacaccagccatgtc

SEQ ID NO: 17 - DNA, 22 bp, artificial
  Reverse primer for generating murine legumain sense riboprobe for in situ hybridization
ctctcagcagtttccccaaatc

SEQ ID NO: 18 - DNA, 313 bp, Mus musculus
acaccaacaccagccatgtcatgcaatatgggaacaaatctatctctaccatgaaagtgatgcagtttcagggaatgaagcacagagccagttcccccatctccctgcctccggtcacacaccttgacctcacccccagccctgacgtgcccctgaccatcttgaagaggaagctgctgagaaccaacgacgtgaaggaatcccagaatctcattgggcagatccagcaatttctggatgccaggcacgtcattgagaagtctgtgcacaagatcgtttccctgctggcgggatttggggaaactgctgagag

SEQ ID NO: 19 - DNA, 20 bp, artificial
  Forward primer for generating murine legumain antisense riboprobe for in situ hybridization
acaccaacaccagccatgtc

SEQ ID NO: 20 - DNA, 51 bp, artificial
  Reverse primer for generating murine legumain antisense riboprobe for in situ hybridization
gactgataatacgactcactatagggcgactctcagcagtttccccaaatc

SEQ ID NO: 21 - DNA, 313 bp, Mus musculus
ctctcagcagtttccccaaatcccgccagcagggaaacgatcttgtgcacagacttctcaatgacgtgcctggcatccagaaattgctggatctgcccaatgagattctgggattccttcacgtcgttggttctcagcagcttcctcttcaagatggtcaggggcacgtcagggctgggggtgaggtcaaggtgtgtgaccggaggcagggagatgggggaactggctctgtgcttcattccctgaaactgcatcactttcatggtagagatagatttgttcccatattgcatgacatggctggtgttggtgt

(19) United States
(12) Patent Application Publication
(10) Pub. No.: US 2007/0274987 A1
(43) Pub. Date: Nov. 29, 2007

(54) Expression of the cysteine protease legumain in vascular and inflammatory diseases

(76) Inventors: Valerie Clerin, Watertown, MA (US); Heather H. Shih, Andover, MA (US); Kathleen Shields, Harvard, MA (US); Jeffrey Feldman, Arlington, MA (US); Gustave T. Hebert, Arlington, MA (US); Debra D. Pittman, Windham, NH (US); Nanhua Dan Deng, Brookline, MA (US)

Correspondence Address: FITZPATRICK CELLA (WYETH), 30 ROCKEFELLER PLAZA, NEW YORK, NY, 10112-3800, US

(21) Appl. No.: 11/806,000

(22) Filed: May 25, 2007

Related U.S. Application Data
(60) Provisional application No. 60/808,381, filed on May 25, 2006.
(60) Provisional application No. 60/837,604, filed on Aug. 15, 2006.

(51) Int. Cl. A61K 39/395; A61K 38/17; C07H 21/04; A61K 39/00; C07K 14/705; C07K 16/28

(52) U.S. Cl. 424/133.1; 435/69.1; 435/320.1; 435/325; 530/395; 514/8; 536/23.5; 530/388.22

(57) ABSTRACT

The present invention provides isolated and purified polynucleotides, polypeptides, and antibodies related to mammalian (e.g., mouse and human) legumain and the novel legumain splice variant, ZB-1. The invention further relates to the use of these isolated and purified polynucleotides, polypeptides, and antibodies, as well as other legumain and ZB-1 agonists and antagonists, in modulating legumain and/or ZB-1 activity, expression, and/or secretion in a cell or cell population, e.g., monocytes, macrophages, foam cells, vascular endothelial cells, kidney proximal tubule cells, arterial endothelial cells, sites of inflammatory cell invasion into a vessel intima, and neointimal lesional areas of an artery. The invention also provides legumain and ZB-1 antagonists, e.g., antagonistic small molecules, antibodies and antibody fragments to legumain and ZB-1, legumain and ZB-1 inhibitory polypeptides, and legumain and ZB-1 inhibitory polynucleotides. The present invention is also directed to novel methods for diagnosing, prognosing, monitoring, treating, ameliorating and/or preventing vascular disorders/diseases and inflammatory disorders/diseases.

### RELATED APPLICATIONS

This application claims the benefit of priority from U.S. Provisional Patent Application Nos. 60/808,381, filed May 25, 2006, and 60/837,604, filed Aug. 15, 2006, the contents of which are hereby incorporated by reference herein in their entireties.

### BACKGROUND OF THE INVENTION

1. Field of the Invention

This invention relates to legumain and the use of legumain in regulating vascular disorders/diseases and inflammatory disorders/diseases. This invention additionally relates to a novel splice variant of legumain, designated ZB-1. The methods and pharmaceutical compositions disclosed herein are useful to diagnose, prognose, monitor, treat, ameliorate and/or prevent vascular disorders/diseases and inflammatory disorders/diseases.

2. Related Background Art

Cysteine proteases (CPs) are a related class of ubiquitous enzymes that are classified in mammals as protein clans based on the structural organization of the enzyme active site (Dickinson (2002) Crit. Rev. Oral Biol. Med. 13:238-75). The mammalian CP clan includes, inter alia, the CA clan, which is comprised of protease members having structural and evolutionary commonality with papain, and the CD clan, which contains caspases and legumain (id.). Legumain, also known as asparaginyl endopeptidase (AEP) or osteoclast inhibitory peptide 2 (OIP-2) (Choi et al. (2001) J. Bone Miner. Res. 16(10):1804-11), is encoded by the PRSC1 gene (Tanaka et al. (1996) Cytogenet. Cell Genet. 74:120-23), and is a relatively new member of the CD clan, with strict specificity for hydrolysis of asparaginyl bonds at the P1 site of the substrate sequence (Chen et al. (1997) J. Biol. Chem. 272:8090-98). Legumain belongs to the C13 family of cysteine proteases that include caspases and separases (Ishii (1994) Methods Enzymol. 244:604-15). Legumain is a unique lysosomal cysteine protease that does not share homology with the papain family of lysosomal proteases to which the cathepsins belong. Under physiological conditions, legumain is present in acidic endosome/lysosome compartments and functions in intracellular protein degradation (Shirahama-Noda et al. (2003) J. Biol. Chem. 278:33194-99). Legumain may play a role in antigen presentation (Manoury et al. (1998) Nature 396:695-99), although legumain-deficient mice do not exhibit defects in antigen presentation of the invariant chain or maturation of class II MHC products (Maehr et al. (2005) J. Immunol. 174:7066-74).

Legumain is a lysosomal endopeptidase that is highly conserved in mammals, with mouse and human legumain displaying about 83% amino acid identity (Chen et al. (1998) J. Biochem. 335:111-17), and human and pig legumain displaying about 84% amino acid identity (Chen et al. (1997) supra).

Legumain protease expression and activity have been evaluated in scores of various tissues (see, e.g., PCT Publication No. WO 05/075675), and found to be detectable in many; high peptidase activity occurs in the kidney (Chen et al. (1998) supra), particularly in kidney proximal tubule cells (Shirahama-Noda et al. (2003) supra). Legumain is additionally expressed in monocytes, where it is believed to play a role in antigen and/or cathepsin L processing (Wolk et al. (2005) Genes Immun. 5:452-56; Maehr et al. (2005) supra; Watts (2005) Immunol. Rev. 207:218-28; Alvarez-Fernandez et al. (1999) J. Biol. Chem. 274:19195-203). Interestingly, the expression of legumain is upregulated during the differentiation of human blood monocytes into dendritic cells, as well as during the activation of human blood macrophages by M-CSF (Li et al. (2003) J. Biol. Chem. 278:38980-90; Hashimoto et al. (1999) Blood 94:837-44). Legumain has also been reported to play a role in osteoclast formation and bone resorption (Choi et al. (1999) J. Biol. Chem. 274:27747-53), endotoxin tolerance (Wolk et al., supra), and epidermal cornification (Zeeuwen et al. (2004) Hum. Mol. Genetics. 13:1069-79). Several protein substrates have been identified for legumain, including MMP2 (Chen et al. (2001) Biol. Chem. 382:777-83), cathepsins H, B, and L (Shirahama-Noda et al. (2003) supra), and α-thymosin (Sarandeses et al. (2003) J. Biol. Chem. 278:13286-93).

Legumain is expressed as a zymogen that is autoactivated by sequential removal of C- and N-terminal propeptides (e.g., cleavage at the N-terminus occurs at residue Asp²⁵ or Asp²¹, while cleavage at the C-terminus occurs at residue Asn³²³) at different pH thresholds, which is believed to be controlled by endosomal acidification or progress through the endosome/lysosome system (Li et al., supra; Kato et al. (2005) Nature Chem. Biol. 1:33-38; Chen et al. (2000) Biochem. J. 352:327-34). The mature legumain protein is additionally N-glycosylated, and displays protease activity that is largely dependent upon low pH, i.e., less than about pH 6.0 (Chen et al. (1997) supra).

Legumain is inhibited by certain cystatins (e.g., ovocystatin and cystatins C and M (see, e.g., PCT Publication No. WO 00/064945 and Vigneswaran et al. (2006) Life Sciences 78:898-907)) and inhibitors of thiol-dependent enzymes (e.g., iodoacetates, iodoacetamides, and maleimides), but is unaffected by the papain inhibitors E64 (trans-epoxysuccinyl-L-leucylamido-(4-guanidino)butane), leupeptin, and Z-Phe-Ala-CHN₂ (id.; Rozman-Pungercar et al. (2003) Cell Death Diff. 10:881-88; Vigneswaran et al., supra; Chen et al. (1998) supra). Certain fluoro- and chloromethylketone peptide caspase inhibitors (such as those disclosed in Rozman-Pungercar et al., supra), and anti-legumain antibodies (Choi et al. (1999) supra) also inhibit legumain activity.

Various synthetic compounds have also been shown to inhibit legumain protease activity, including aza-peptide Micheal acceptors/inhibitors (Niestroj et al. (2002) Biol. Chem. 383:1205-14; Ekici et al. (2004) J. Med. Chem. 47:1889-92; Götz (2004) “Design, Synthesis and Evaluation of Irreversible Peptidyl Inhibitors for Clan CA and Clan CD Cysteine Proteases” Thesis Dissertation, May 2004, Georgia Institute of Technology), aza-peptide epoxides (Götz, supra; Asgian et al. (2002) J. Med. Chem. 45:4958-60; James et al. (2003) Biol. Chem. 384:1613-18; U.S. Pat. No. 7,056,947), methylketones (such as acyloxymethylketones, e.g., 2,6-dimethyl-benzoic acid 3-benzyloxycarbonylamino-4-carbamoyl-2-oxo-butyl ester [MV026630] as disclosed in Loak et al. (2003) Biol. Chem. 384:1239-46, and halomethylketones, e.g., those disclosed in Niestroj et al., supra), and other synthetics (see, e.g., U.S. Pat. No. 6,004,933; PCT Publication Nos. WO 03/016335 and WO 99/048910; Yamane et al. (2002) Biochim. Biophys. Acta 1596:108-20 (disclosing inhibition of legumain by p-chloromercuribenzene-sulfonic acid, Hg2+, and Cu2+); and Li et al., supra (disclosing the reversible AEP inhibitor F_{moc}-AENK-amide)).

Atherosclerosis is a generalized and inflammatory vascular disease of the arterial blood vessel, commonly referred to as “hardening” of the arteries, which results from fat deposition inside the vessel wall. The initial step of atherogenesis is the formation of fatty streaks, which are largely comprised of foam cells, i.e., macrophage cells filled with massive amounts of phagocytosed cholesterol (e.g., Greaves and Gordon (2005) J. Lipid Res. 46:11-20). It is believed that these streaks are initiated by the adherence of monocytes to activated endothelial cells in the arterial cell walls (id.). Adherent monocytes then migrate from the vessel lumen into the subendothelial space of the vessel intima (i.e., the neointima), in the process known as extravasation, where they differentiate into macrophages that recognize and engulf low-density lipoproteins (LDLs) via scavenger receptors such as CD36 and SR-A (Wasserman and Shipley (2006) Mt. Sinai J. Med. 73:431-39; Lucas and Greaves (2001) Expert Rev. Mol. Med. 5:1-18). Over time, smooth muscle cells of the vessel media also begin to proliferate and migrate into the neointima where they accumulate cholesterol, becoming smooth muscle-derived foam cells (id.). Both smooth muscle-derived and macrophage-derived foam cells eventually necrose, leaving a lipid-filled core that is enriched with matrix molecules and cellular debris, and which is walled-off from the lumen of the artery by a matrix cap secreted by the remaining smooth muscle cells (id.). The resultant structure is an atherosclerotic lesion, which is covered by a fibrous “atherosclerotic” or “atheromatous” plaque.

Because the inelastic atheromatous plaque thickens the vessel wall, thereby decreasing the arterial lumenal diameter, the artery expands in size, resulting in arterial aneurysms (Wasserman and Shipley, supra; Stary et al. (1995) Circulation 92:1355-74). If the expansion is insufficient to expand the lumen of the artery in relation to the thickening of the artery wall, stenosis results (id.). Moreover, the thinner and weaker fibrous caps (i.e., “vulnerable” or “unstable” caps) often rupture (Wasserman and Shipley, supra). During plaque rupture, inflammatory cells localize to the shoulder region of the vulnerable plaque (Lucas and Greaves, supra). In this area of the lesion, T lymphocytes (CD4⁺) secrete IFNγ, an inflammatory cytokine that impairs vascular smooth muscle cell proliferation and collagen synthesis, which weakens the atheromatous plaque (id.). In addition, activated macrophages within the lesion produce matrix metalloproteinases (MMPs) that degrade collagen (id.). These mechanisms highlight the role of inflammatory cells in the denudation and rupture of the fibrous cap.

Plaque rupture may result in thrombosis due to platelet aggregation at the rupture site, partial or complete occlusion of the blood vessel, and progression of the atherosclerotic lesion due to incorporation of the thrombus into the atherosclerotic plaque. Thrombus formation and accumulation in the artery enhances the stenosis already induced by the presence of the atheromatous plaque, resulting in obstruction of blood flow (i.e., ischemia or stroke) to downstream tissues, such as heart or kidney (id.). Platelet-derived growth factor (PDGF), insulin-like growth factor (IGF), transforming growth factor (TGF) alpha and beta, macrophage colony stimulating factor (M-CSF), thrombin, macrophage chemoattractant protein-1 (MCP-1), and angiotensin II are mitogens produced by activated platelets, macrophages, and activated endothelial cells at sites of endothelial cell disruption that characterize early atherogenesis, vascular inflammation, and atherothrombosis (id.).

Proteolysis is a pathological event involved in multiple aspects of atherogenesis, including the infiltration of leukocytes into subendothelial space, the migration of SMCs into the intima, the degradation of the extracellular matrix and destabilization of the plaque, and neovascularization (Liu et al. (2004) Arterioscler. Thromb. Vasc. Biol. 24:1359-66). A number of proteases have been implicated in the development of atherosclerosis. In addition to metalloproteases (MMPs) and serine proteases, the lysosomal cysteine proteases have recently been linked to atherogenesis. For example, deletion of cathepsin S, K or L led to reduced atherosclerosis in LDLR−/− or ApoE−/− mice, demonstrating a functional role for these cysteine proteases in atherogenesis (Sukhova et al. (2003) J. Clin. Invest. 111(6):897-906; Lutgens et al. (2006) Circulation 113(1):98-107; Kitamoto et al. (2007) Circulation 115(15):2065-75). Recently, the legumain gene was found to be differentially expressed in stable and in unstable human atherosclerotic plaques (Papaspyridonos et al. (2006) Arterioscler. Thromb. Vasc. Biol. 26:1837-44.).

### SUMMARY OF THE INVENTION

The present invention provides various methods and compositions related to mammalian legumains, e.g., human, mouse, and pig legumain, and mammalian legumain splice variants, particularly the novel splice variant ZB-1. In the present study, the inventors document the gene and protein expression of legumain in mouse models of atherosclerosis and further characterize legumain expression in human atherosclerotic tissue. In addition, the inventors report that macrophage-expressed legumain may contribute to atherogenesis via protease-dependent as well as protease-independent mechanisms.

In at least one embodiment, the invention disclosed herein provides a polynucleotide comprising the nucleic acid sequence set forth in SEQ ID NO:11. In another embodiment, the polynucleotide comprises a nucleic acid sequence that hybridizes under high stringency conditions to the nucleic acid sequence or the complement of the nucleic acid sequence set forth in SEQ ID NO:11. In another embodiment, the invention provides a polynucleotide comprising a nucleic acid sequence that encodes an amino acid sequence selected from the group consisting of the amino acid sequence set forth in SEQ ID NO:12, amino acids 21 to 323 of SEQ ID NO:12, amino acids 25 to 323 of SEQ ID NO:12, and other active fragments of SEQ ID NO:12. In another embodiment, the polynucleotide comprises a nucleic acid sequence that hybridizes under high stringency conditions to a nucleic acid sequence or a complement of a nucleic acid sequence that encodes an amino acid sequence selected from the group consisting of the amino acid sequence set forth in SEQ ID NO:12, amino acids 21 to 323 of SEQ ID NO:12, amino acids 25 to 323 of SEQ ID NO:12, and other active fragments of SEQ ID NO:12. In other embodiments, a polynucleotide with a high sequence identity to one or more of these sequences is provided.

In at least one embodiment, the invention disclosed herein provides a polypeptide comprising the amino acid sequence set forth in SEQ ID NO:12, amino acids 21 to 323 of SEQ ID NO:12, or amino acids 25 to 323 of SEQ ID NO:12. In another embodiment, the invention provides a polypeptide encoded by the nucleic acid sequence set forth in SEQ ID NO:1. In another embodiment, the invention provides a polypeptide encoded by a nucleic acid sequence that hybridizes under high stringency conditions to the complement of the nucleic acid sequence set forth in SEQ ID NO:11. In other embodiments, a polypeptide with a high sequence identity to one or more of these sequences is provided.

In at least one embodiment, the invention disclosed herein provides an antibody or antigen binding fragment thereof that specifically binds a mammalian ZB-1 polypeptide or a fragment of a mammalian ZB-1 polypeptide. In another embodiment, the mammalian ZB-1 polypeptide or the fragment of a mammalian ZB-1 polypeptide is derived from a human. In another embodiment, the human ZB-1 polypeptide comprises the amino acid sequence set forth in SEQ ID NO:12 or an active fragment of the amino acid sequence set forth in SEQ ID NO:12. In another embodiment, the antibody or antigen binding fragment thereof is antagonistic or agonistic.

In at least one embodiment, the invention disclosed herein provides a pharmaceutical composition comprising a therapeutically effective amount of ZB-1 and a pharmaceutically acceptable carrier.

In at least one embodiment, the invention disclosed herein provides the use of a legumain antagonist and/or a ZB-1 antagonist for the preparation of a pharmaceutical composition for use in a method of treating, ameliorating, or preventing a vascular disorder or an inflammatory disorder, wherein the pharmaceutical composition comprises a therapeutically effective amount of the legumain antagonist and/or the ZB-1 antagonist, and a pharmaceutically acceptable carrier. In another embodiment, the disorder is an inflammatory disorder. In another embodiment, the inflammatory disorder is selected from the group consisting of arthritis, tuberculosis, multiple sclerosis, Crohn's disease, or ulcerative colitis. In another embodiment, the disorder is a vascular disorder. In another embodiment, the vascular disorder is selected from the group consisting of atherosclerosis, congestive heart failure, myocardial infarction, arrhythmia, atrial arrhythmia, ventricular arrhythmia, stenosis, aneurysm, peripheral vascular disease, peripheral arterial disease, chronic peripheral arterial occlusive disease, thrombosis, atherothrombosis, deep venous thrombosis, acute arterial thrombosis, embolism, inflammatory vascular disorders, Raynaud's phenomenon, vasculitis, arteritis, venous disorders, hypertensive vascular disease, claudication, angina, stable angina, unstable angina, stroke, peripheral artery occlusive disease, coronary artery disease, acute coronary syndrome, metabolic syndrome, ischemia, reperfusion, chronic kidney disease, end-stage renal disease, diabetic nephropathy, hyperlipidemia, hypertension, and diabetes. In another embodiment, the legumain antagonist and/or ZB-1 antagonist is selected from the group consisting of inhibitory polynucleotides, inhibitory polypeptides, small molecules, antagonistic antibodies and antigen binding fragments thereof. In another embodiment, the inhibitory polynucleotide is selected from the group consisting of antisense polynucleotides, siRNA molecules, ribozymes, and aptamers. In another embodiment, the inhibitory polypeptide is selected from the group consisting of cystatins or active fragments thereof, aza-peptide Micheal acceptors/inhibitors, aza-peptide epoxides, fluoromethylketone peptide caspase inhibitors, and chloromethylketone peptide caspase inhibitors. In another embodiment, the small molecule is selected from the group consisting of methylketones, iodoacetates, iodoacetamides, and maleimides.

In at least one embodiment, the invention disclosed herein provides a method for treating, ameliorating, or preventing a vascular disorder or an inflammatory disorder in a mammal comprising administering to the mammal a therapeutically effective amount of a legumain antagonist and/or a ZB-1 antagonist. In another embodiment, the disorder is an inflammatory disorder. In another embodiment, the inflammatory disorder is selected from the group consisting of arthritis, tuberculosis, multiple sclerosis, Crohn's disease, or ulcerative colitis. In another embodiment, the disorder is a vascular disorder. In another embodiment, the vascular disorder is selected from the group consisting of atherosclerosis, congestive heart failure, myocardial infarction, arrhythmia, atrial arrhythmia, ventricular arrhythmia, stenosis, aneurysm, peripheral vascular disease, peripheral arterial disease, chronic peripheral arterial occlusive disease, thrombosis, atherothrombosis, deep venous thrombosis, acute arterial thrombosis, embolism, inflammatory vascular disorders, Raynaud's phenomenon, vasculitis, arteritis, venous disorders, hypertensive vascular disease, atherothrombosis, claudication, angina, stable angina, unstable angina, stroke, peripheral artery occlusive disease, coronary artery disease, acute coronary syndrome, metabolic syndrome, ischemia, reperfusion, chronic kidney disease, end-stage renal disease, diabetic nephropathy, hyperlipidemia, hypertension, and diabetes. In another embodiment, the legumain antagonist and/or ZB-1 antagonist is selected from the group consisting of inhibitory polynucleotides, inhibitory polypeptides, small molecules, antagonistic antibodies and antigen binding fragments thereof. In another embodiment, the inhibitory polynucleotide is selected from the group consisting of antisense polynucleotides, siRNA molecules, ribozymes, and aptamers. In another embodiment, the inhibitory polypeptide is selected from the group consisting of cystatins or active fragments thereof, aza-peptide Micheal acceptors/inhibitors, aza-peptide epoxides, fluoromethylketone peptide caspase inhibitors, and chloromethylketone peptide caspase inhibitors. In another embodiment, the small molecule is selected from the group consisting of methylketones, iodoacetates, iodoacetamides, and maleimides.

In at least one embodiment, the invention disclosed herein provides a method for treating, ameliorating, or preventing a vascular disorder or an inflammatory disorder in a mammal comprising contacting a cell or cell population of the mammal with a therapeutically effective amount of a legumain antagonist and/or a ZB-1 antagonist. In another embodiment, the cell or cell population comprises a macrophage, a monocyte, a vascular endothelial cell, a foam cell, or a mixture of monocytes, macrophages, vascular endothelial cells and/or foam cells. In another embodiment, the cell or cell population secretes legumain and/or ZB-1. In another embodiment, the cell or cell population comprises an arterial endothelial cell or a kidney proximal tubule cell. In another embodiment, the cell or cell population is derived from a site of inflammatory cell infiltration into the intima of an artery. In another embodiment, the cell or cell population is derived from a neointimal lesional area of an artery.

In at least one embodiment, the invention disclosed herein provides a method for decreasing the level of legumain and/or ZB-1 activity, expression, and/or secretion in a cell or cell population, comprising contacting the cell or cell population with a legumain antagonist and/or a ZB-1 antagonist in an amount sufficient to decrease the level of activity, expression, and/or secretion of legumain and/or ZB-1 in the cell or cell population. In another embodiment, the cell or cell population comprises a macrophage, a monocyte, a vascular endothelial cell, a foam cell, or a mixture of monocytes, macrophages, vascular endothelial cells and/or foam cells. In another embodiment, the cell or cell population secretes legumain and/or ZB-1. In another embodiment, the cell or cell population comprises an arterial endothelial cell or a kidney proximal tubule cell. In another embodiment, the cell or cell population is derived from a site of inflammatory cell infiltration into the intima of an artery. In another embodiment, the cell or cell population is derived from a neointimal lesional area of an artery.

In at least one embodiment, the invention disclosed herein provides a method for decreasing the level of legumain and/or ZB-1 activity, expression, and/or secretion in a mammal comprising administering to the mammal a legumain antagonist and/or a ZB-1 antagonist in an amount sufficient to decrease the level of activity, expression, and/or secretion of legumain and/or ZB-1 in the mammal. In another embodiment, the legumain antagonist and/or ZB-1 antagonist is selected from the group consisting of inhibitory polynucleotides, inhibitory polypeptides, small molecules, antagonistic antibodies and antigen binding fragments thereof. In another embodiment, the inhibitory polynucleotide is selected from the group consisting of antisense polynucleotides, siRNA molecules, ribozymes, and aptamers. In another embodiment, the inhibitory polypeptide is selected from the group consisting of cystatins or active fragments thereof, aza-peptide Micheal acceptors/inhibitors, aza-peptide epoxides, fluoromethylketone peptide caspase inhibitors, and chloromethylketone peptide caspase inhibitors. In another embodiment, the small molecule is selected from the group consisting of methylketones, iodoacetates, iodoacetamides, and maleimides.

In at least one embodiment, the invention disclosed herein provides a method for monitoring the course of a treatment for a vascular disorder or inflammatory disorder in a patient, comprising: measuring the level of activity, expression and/or secretion of legumain and/or ZB-1 in a cell or cell population from the patient; administering a legumain antagonist and/or a ZB-1 antagonist to the patient; and measuring the level of activity, expression and/or secretion of legumain and/or ZB-1 in a cell or cell population from the patient following administration of the legumain antagonist and/or ZB-1 antagonist, wherein a lower level of activity, expression and/or secretion of legumain and/or ZB-1 in the cell or cell population from the patient following administration of the legumain antagonist and/or ZB-1 antagonist, in comparison to the level of activity, expression and/or secretion of legumain and/or ZB-1 in the cell or cell population from the patient prior to administration of the legumain antagonist and/or ZB-1 antagonist, provides a positive indication of the effect of the treatment for the vascular disorder or inflammatory disorder in the patient.

In at least one embodiment, the invention disclosed herein provides a method for monitoring a vascular disorder or inflammatory disorder in a patient, comprising: measuring the level of activity, expression and/or secretion of legumain and/or ZB-1 in a cell or cell population from the patient at a first time point; and measuring the level of activity, expression and/or secretion of legumain and/or ZB-1 in a cell or cell population from the patient at a second time point, wherein a lower level of activity, expression and/or secretion of legumain and/or ZB-1 in the cell or cell population from the patient at the second time point, in comparison to the level of activity, expression and/or secretion of legumain and/or ZB-1 in the cell or cell population from the patient at the first time point, provides an indication that the vascular disorder or inflammatory disorder has decreased in severity.

In at least one embodiment, the invention disclosed herein provides a method for monitoring a vascular disorder or inflammatory disorder in a patient, comprising: measuring the level of activity, expression and/or secretion of legumain and/or ZB-1 in a cell or cell population from the patient; and comparing the level of activity, expression and/or secretion of legumain and/or ZB-1 in the cell or cell population from the patient to the level of activity, expression and/or secretion of legumain and/or ZB-1 in a reference cell or cell population, wherein a lower level of activity, expression and/or secretion of legumain and/or ZB-1 in the cell or cell population from the patient, in comparison to the level of activity, expression and/or secretion of legumain and/or ZB-1 in the reference cell or cell population, provides an indication that the vascular disorder or inflammatory disorder has decreased in severity.

In at least one embodiment, the invention disclosed herein provides a method for prognosing a vascular disorder or inflammatory disorder in a patient, comprising: measuring the level of activity, expression and/or secretion of legumain and/or ZB-1 in a cell or cell population from the patient at a first time point; and measuring the level of activity, expression and/or secretion of legumain and/or ZB-1 in a cell or cell population from the patient at a second time point, wherein a lower level of activity, expression and/or secretion of legumain and/or ZB-1 in the cell or cell population from the patient at the second time point, in comparison to the level of activity, expression and/or secretion of legumain and/or ZB-1 in the cell or cell population from the patient at the first time point, indicates a decreased likelihood that the patient either will develop the vascular disorder or inflammatory disorder, or will develop a more severe form of the vascular disorder or inflammatory disorder.

In at least one embodiment, the invention disclosed herein provides a method for prognosing a vascular disorder or inflammatory disorder in a patient, comprising: measuring the level of activity, expression and/or secretion of legumain and/or ZB-1 in a cell or cell population from the patient; and comparing the level of activity, expression and/or secretion of legumain and/or ZB-1 in the cell or cell population to the level of activity, expression and/or secretion of legumain and/or ZB-1 in a reference cell or cell population, wherein a lower level or similar level of activity, expression and/or secretion of legumain and/or ZB-1 in the cell or cell population from the patient, in comparison to the level of activity, expression and/or secretion of legumain and/or ZB-1 in the reference cell or cell population, indicates a decreased likelihood that the patient either will develop the vascular disorder or inflammatory disorder, or will develop a more severe form of the vascular disorder or inflammatory disorder.

In at least one embodiment, the invention disclosed herein provides a method of screening for a compound capable of treating, ameliorating, or preventing a vascular disorder or an inflammatory disorder comprising the steps of: contacting a sample containing legumain and/or ZB-1 with a compound of interest; and determining whether the level of activity, expression, and/or secretion of legumain and/or ZB-1 in the contacted sample is decreased relative to the level of activity, expression, and/or secretion of legumain and/or ZB-1 in a sample not contacted with the compound, wherein a decrease in the level of activity, expression, and/or secretion of legumain and/or ZB-1 in the contacted sample identifies the compound as a compound that is capable of treating, ameliorating, or preventing a vascular disorder or an inflammatory disorder.

In at least one embodiment, the invention disclosed herein provides a method for treating, ameliorating, or preventing a vascular disorder or an inflammatory disorder in a mammal comprising administering to the mammal a therapeutically effective amount of a legumain agonist and/or a ZB-1 agonist. In another embodiment, the invention provides the use of a legumain agonist and/or a ZB-1 agonist for the preparation of a pharmaceutical composition for use in a method of treating, ameliorating, or preventing a vascular disorder or an inflammatory disorder, wherein the pharmaceutical composition comprises a therapeutically effective amount of the legumain agonist and/or the ZB-1 agonist, and a pharmaceutically acceptable carrier.

In at least one embodiment, the invention disclosed herein provides a method for inhibiting cell migration in a mammal comprising administering to the mammal a legumain antagonist and/or a ZB-1 antagonist. In another embodiment, the legumain antagonist and/or ZB-1 antagonist is selected from the group consisting of inhibitory polynucleotides, inhibitory polypeptides, small molecules, antagonistic antibodies and antigen binding fragments thereof. In another embodiment, the inhibitory polynucleotide is selected from the group consisting of antisense polynucleotides, siRNA molecules, ribozymes, and aptamers. In another embodiment, the inhibitory polypeptide is selected from the group consisting of cystatins or active fragments thereof, aza-peptide Micheal acceptors/inhibitors, aza-peptide epoxides, fluoromethylketone peptide caspase inhibitors, and chloromethylketone peptide caspase inhibitors. In another embodiment, the small molecule is selected from the group consisting of methylketones, iodoacetates, iodoacetamides, and maleimides. In another embodiment, the invention provides the use of a legumain antagonist and/or a ZB-1 antagonist for the preparation of a pharmaceutical composition for use in a method of inhibiting cell migration in a mammal, wherein the pharmaceutical composition comprises a therapeutically effective amount of the legumain antagonist and/or the ZB-1 antagonist, and a pharmaceutically acceptable carrier.

In at least one embodiment, the invention disclosed herein provides a method for promoting cell migration in a mammal comprising administering to the mammal a legumain agonist and/or a ZB-1 agonist. In another embodiment, the invention provides the use of a legumain agonist and/or a ZB-1 agonist for the preparation of a pharmaceutical composition for use in a method of cell migration in a mammal, wherein the pharmaceutical composition comprises a therapeutically effective amount of the legumain agonist and/or the ZB-1 agonist, and a pharmaceutically acceptable carrier.

In at least one embodiment, the invention disclosed herein provides a method of promoting wound healing in a mammal comprising administering to the mammal a legumain agonist and/or a ZB-1 agonist. In another embodiment, the invention provides the use of a legumain agonist and/or a ZB-1 agonist for the preparation of a pharmaceutical composition for use in a method of promoting wound healing in a mammal, wherein the pharmaceutical composition comprises a therapeutically effective amount of the legumain agonist and/or the ZB-1 agonist, and a pharmaceutically acceptable carrier.

In at least one embodiment, the invention disclosed herein provides a method for inhibiting angiogenesis in a mammal comprising administering to the mammal a legumain antagonist and/or a ZB-1 antagonist. In another embodiment, the legumain antagonist and/or ZB-1 antagonist is selected from the group consisting of inhibitory polynucleotides, inhibitory polypeptides, small molecules, antagonistic antibodies and antigen binding fragments thereof. In another embodiment, the inhibitory polynucleotide is selected from the group consisting of antisense polynucleotides, siRNA molecules, ribozymes, and aptamers. In another embodiment, the inhibitory polypeptide is selected from the group consisting of cystatins or active fragments thereof, aza-peptide Micheal acceptors/inhibitors, aza-peptide epoxides, fluoromethylketone peptide caspase inhibitors, and chloromethylketone peptide caspase inhibitors. In another embodiment, the small molecule is selected from the group consisting of methylketones, iodoacetates, iodoacetamides, and maleimides. In another embodiment, the invention provides the use of a legumain antagonist and/or a ZB-1 antagonist for the preparation of a pharmaceutical composition for use in a method of inhibiting angiogenesis in a mammal, wherein the pharmaceutical composition comprises a therapeutically effective amount of the legumain antagonist and/or the ZB-1 antagonist, and a pharmaceutically acceptable carrier.

In at least one embodiment, the invention disclosed herein provides a method for promoting angiogenesis in a mammal comprising administering to the mammal a legumain agonist and/or a ZB-1 agonist. In another embodiment, the invention provides the use of a legumain agonist and/or a ZB-1 agonist for the preparation of a pharmaceutical composition for use in a method of promoting angiogenesis in a mammal, wherein the pharmaceutical composition comprises a therapeutically effective amount of the legumain agonist and/or the ZB-1 agonist, and a pharmaceutically acceptable carrier.

In at least one embodiment, the invention disclosed herein provides a method for inhibiting proliferation of endothelial cells in a mammal comprising administering to the mammal a legumain antagonist and/or a ZB-1 antagonist. In another embodiment, the legumain antagonist and/or ZB-1 antagonist is selected from the group consisting of inhibitory polynucleotides, inhibitory polypeptides, small molecules, antagonistic antibodies and antigen binding fragments thereof. In another embodiment, the inhibitory polynucleotide is selected from the group consisting of antisense polynucleotides, siRNA molecules, ribozymes, and aptamers. In another embodiment, the inhibitory polypeptide is selected from the group consisting of cystatins or active fragments thereof, aza-peptide Micheal acceptors/inhibitors, aza-peptide epoxides, fluoromethylketone peptide caspase inhibitors, and chloromethylketone peptide caspase inhibitors. In another embodiment, the small molecule is selected from the group consisting of methylketones, iodoacetates, iodoacetamides, and maleimides. In another embodiment, the invention provides the use of a legumain antagonist and/or a ZB-1 antagonist for the preparation of a pharmaceutical composition for use in a method of inhibiting proliferation of endothelial cells in a mammal, wherein the pharmaceutical composition comprises a therapeutically effective amount of the legumain antagonist and/or the ZB-1 antagonist, and a pharmaceutically acceptable carrier.

In at least one embodiment, the invention disclosed herein provides a method of promoting proliferation of endothelial cells in a mammal comprising administering to the mammal a legumain agonist and/or a ZB-1 agonist. In another embodiment, the invention provides the use of a legumain agonist and/or a ZB-1 agonist for the preparation of a pharmaceutical composition for use in a method of promoting proliferation of endothelial cells in a mammal, wherein the pharmaceutical composition comprises a therapeutically effective amount of the legumain agonist and/or the ZB-1 agonist, and a pharmaceutically acceptable carrier.

In at least one embodiment, the invention disclosed herein provides a method of inhibiting tumor metastasis, comprising contacting a cell or cell population of the mammal with a legumain antagonist and/or ZB-1 antagonist. In at least one other embodiment, the invention disclosed herein provides a method of inhibiting tumor metastasis in a mammal comprising administering to the mammal a legumain antagonist and/or ZB-1 antagonist. In another embodiment, the legumain antagonist and/or ZB-1 antagonist is selected from the group consisting of inhibitory polynucleotides, inhibitory polypeptides, small molecules, antagonistic antibodies and antigen binding fragments thereof. In another embodiment, the inhibitory polynucleotide is selected from the group consisting of antisense polynucleotides, siRNA molecules, ribozymes, and aptamers. In another embodiment, the inhibitory polypeptide is selected from the group consisting of cystatins or active fragments thereof, aza-peptide Micheal acceptors/inhibitors, aza-peptide epoxides, fluoromethylketone peptide caspase inhibitors, and chloromethylketone peptide caspase inhibitors. In another embodiment, the small molecule is selected from the group consisting of methylketones, iodoacetates, iodoacetamides, and maleimides. In another embodiment, the invention provides the use of a legumain antagonist and/or a ZB-1 antagonist for the preparation of a pharmaceutical composition for use in a method of inhibiting tumor metastasis in a mammal, wherein the pharmaceutical composition comprises a therapeutically effective amount of the legumain antagonist and/or the ZB-1 antagonist, and a pharmaceutically acceptable carrier.

In at least one embodiment, the invention disclosed herein provides a method of promoting transplant surgery recovery, comprising contacting a cell or cell population of the mammal with a legumain agonist and/or a ZB-1 agonist. In another embodiment, the invention provides the use of a legumain agonist and/or a ZB-1 agonist for the preparation of a pharmaceutical composition for use in a method of promoting transplant surgery recovery in a mammal, wherein the pharmaceutical composition comprises a therapeutically effective amount of the legumain agonist and/or the ZB-1 agonist, and a pharmaceutically acceptable carrier.

In at least one embodiment, the invention disclosed herein provides a method for treating, ameliorating, or preventing a vascular disorder or an inflammatory disorder in a mammal comprising administering to the mammal a therapeutically effective amount of OIP-2. In another embodiment, the invention provides the use of OIP-2 for the preparation of a pharmaceutical composition for use in a method of treating, ameliorating, or preventing a vascular disorder or an inflammatory disorder, wherein the pharmaceutical composition comprises a therapeutically effective amount of OIP-2 and a pharmaceutically acceptable carrier.

### BRIEF DESCRIPTION OF THE DRAWINGS

FIG. 1 shows legumain mRNA expression (upper panel; Y-axis: “Normalized Intensity (fold scale)”) in human atherosclerotic arterial samples with plaques (X-axis: “Athero Plaque”) or plaque-free (X-axis: “Athero Vessel”) segments compared to healthy arterial samples (X-axis: “Normal”). Legumain (201212_at) expression is increased in atherosclerotic arterial samples containing plaques relative to plaque-free segments or nondiseased arterial samples; values are shown in the lower panel.

FIG. 2 shows legumain mRNA expression (Y-axis: “Frequency (ppm)”) in the aortic arch of ApoE−/− mice (“ApoE−/−”) and C57BL/6 wild-type control mice (“C57BL/6”) at 5 to 55 weeks of age (X-axis: “Age (weeks)”). Legumain gene expression increases with progression of disease.

FIG. 3 shows validation of legumain mRNA expression (Y-axis: “Relative TAQMAN® Units (β-Actin Normalized)”) profile in atherosclerosis by Real-Time PCR (RT-PCR). mRNA was isolated from the aortic arch of ApoE−/− mice (“ApoE−/−”) and C57BL/6 wild-type control mice (“C57BL/6”) at 40 weeks at 12 to 54 weeks of age (X-axis: Age (weeks)) and analyzed by RT-PCR. The data demonstrate that legumain gene expression increases as atherosclerosis progresses in mice.

FIG. 4 shows that legumain mRNA, protein, and activity are increased in differentiated (macrophage) THP1 human monocytic leukemia cells. (FIG. 4A) THP1 cells were differentiated in PMA (phorbol 12-myristate 13-acetate)-containing media for 0, 1, 2, or 3 days (X-axis: “Days after differentiation”). The mRNA levels (Y-axis: “Fold Change”) were determined by TAQMAN® (Applied Biosystems, Foster City, Calif.) quantitative-PCR. (FIG. 4B) Mature legumain and actin (control) protein levels in undifferentiated (left lane) or PMA-differentiated THP1 cells (right lane) were determined by Western analysis using an anti-legumain polyclonal antibody and an anti-actin polyclonal antibody, respectively. Molecular weight markers (in kDa) are shown on the left side. (FIG. 4C) Using the same cell lysates as in FIG. 4B, the protease activity of legumain (Y-axis: “Rate of Reaction”) was determined by measuring the hydrolysis of the legumain substrate peptide, Z-AAN-MCA (Peptide Institute, Louisville, Ky.) (X-axis: undifferentiated THP1 monocytes, “Non-dif;” differentiated THP1 macrophages, “Dif”).

FIG. 5 shows that legumain protein levels and activity are increased in M-CSF-activated primary human macrophages. Cell lysates were prepared from primary human macrophages cultured in serum-free media (FIGS. 5A and 5B) or RPMI media supplemented with 0.25% FBS (FIG. 5C) with or without M-CSF. (FIG. 5A): Detection of legumain protein (“Mature legumain” and “Pro-legumain”) in M-CSF-stimulated human macrophages by Western analysis. Lane 1: unstimulated; Lane 2: M-CSF treated. “Actin”: actin loading control. (FIG. 5B): Detection of pro-legumain in conditioned media from M-CSF stimulated human macrophages. Lane 1: unstimulated; Lane 2: M-CSF treated. (FIG. 5C): Detection of legumain activity (Y-axis: “Rate of Reaction”) in M-CSF-stimulated human macrophages (X-axis: “M-CSF”) or unstimulated macrophages (X-axis: “un-treated”) measured by the hydrolysis of Z-AAN-MCA.

FIG. 6 shows dose-dependent migration of primary human monocytes towards purified legumain in Boyden chambers, measured and quantified using a luminescent cell viability assay (Y-axis: “Luminescence”). VEGF at 10 ng/mL and 5% FBS were used as positive controls; statistical significance was achieved at p<0.05 (ANOVA). An asterisk (*) denoted significance compared to 0 ng/ml.

FIG. 7 shows detection of cell-surface legumain activity. 293 cells were infected with adenovirus expressing mouse legumain. Cell-surface legumain activity (Y-axis: “Rate of Reaction”) was determined by incubating legumain-expressing cells in a reaction buffer containing the legumain substrate peptide Z-AAN-MCA (Peptide Institute) in the presence or absence of protease inhibitors cystatin C or E64. Control: “No inhibitor.”

FIG. 8 shows the effect of legumain on HEK293 cell migration in the in vitro wound-healing assay. VEGF (10 ng/mL) and 5% FBS were used as positive controls. The results reveal a significant increase in cell migration in response to stimulation with legumain at 10 ng/mL and 25 ng/mL relative to control, denoted by an asterisk (*). Statistical significance is achieved at p<0.05 (ANOVA).

FIG. 9 shows the effect of legumain on human umbilical vein endothelial cell (HUVEC) migration in the in vitro wound-healing assay. VEGF (10 ng/mL) and 5% FBS were used as positive controls. The results reveal a significant increase in cell migration in response to stimulation with legumain at 25 ng/mL relative to control, denoted by an asterisk (*). Legumain at 25 ng/mL promotes an increase in migration to the same extent as VEGF at 10 ng/mL. Statistical significance is achieved at p<0.05 (ANOVA).

FIG. 10 shows the dose-dependent Matrigel invasion of HUVECs exposed to purified legumain in modified Boyden chambers, measured and quantified using a luminescent cell viability assay (Y-axis: “Luminescence”). VEGF at 10 ng/mL was used as a positive control. Legumain loaded in top and bottom chambers at 25 ng/mL was used as a negative control. The results reveal a significant increase in cell invasion in response to stimulation with legumain at 25 ng/mL relative to control, denoted by an asterisk (*). Legumain at 25 ng/mL promotes an increase in invasion to the same extent as VEGF at 10 ng/mL. Statistical significance is achieved at p<0.05 (ANOVA).

FIG. 11 shows the amino acid sequence of human ZB-1 (bottom sequence (1-377)) aligned with the human legumain sequence (top sequence (1-434)). “Asp²⁵ Cleavage”: N-terminal propeptide cleavage site; “Asn³²³ Cleavage”: C-terminal propeptide cleavage site.

### DETAILED DESCRIPTION OF THE INVENTION

The findings disclosed herein identify legumain as a gene involved in vascular disorders, e.g., cardiovascular disorders, such as atherosclerosis, and inflammatory disorders, e.g., chronic inflammatory disorders, such as arthritis.

Additionally disclosed herein is a novel splice variant of legumain, designated ZB-1, which lacks amino acids 341-397 of full-length human legumain, and which, like legumain, is secreted upon overexpression in cell culture. Thus, ZB-1 may also function in vascular and inflammatory disorders.

Disclosed herein are the findings that: (1) legumain mRNA and protein expression increase dramatically during lesion formation in mouse models of atherosclerosis; (2) legumain mRNA is increased in human atherosclerotic samples compared to nondiseased tissues; (3) legumain protein is detected in the foam cells of atherosclerotic plaques of ApoE−/− mice; (4) legumain is expressed by arterial endothelial cells of aortic sinus in ApoE−/− mice; (5) legumain is highly expressed in activated human macrophages and differentiated macrophages, and is released into the culture media of activated human macrophages; (6) legumain activity is markedly increased in cell culture during macrophage differentiation and macrophage activation; (7) enzymatically active legumain is detected on the cell surface of recombinant legumain-overexpressing cells; (8) legumain is expressed in the kidney, e.g., in endothelial cells of renal arteries, and in proximal tubule cells; (9) legumain is expressed in coronary arteries of an atherosclerotic patient; (10) legumain stimulation induces human monocyte migration; and (11)) legumain stimulation induces endothelial cell migration and proliferation in wound-healing models of HEK293 and HUVEC cultures, as well as endothelial cell invasion in HUVEC culture. Taken together, these findings implicate a functional link between legumain (and ZB-1) and vascular and/or inflammatory diseases, e.g., (i.e., including but not limited to) atherosclerosis.

Also disclosed herein is the finding that legumain expression is increased in the collagen-induced arthritic (CIA) paw in a mouse model of arthritis. As monocyte recruitment and macrophage differentiation, which occur during e.g., atherogenesis (a form of vascular inflammation), are also typical features of diseases characterized by chronic inflammation (e.g., arthritis and tuberculosis), these findings suggest that legumain and ZB-1 may have general roles in inflammatory diseases.

In light of these findings, assaying and/or modulating the secretion, expression, and/or activity of legumain and legumain variants, such as the novel ZB-1, provide excellent tools for diagnosing, prognosing, monitoring, treating, ameliorating and/or preventing vascular disorders and inflammatory disorders, and disorders associated therewith.

As such, the present invention provides legumain and ZB-1 modulators (e.g., legumain and ZB-1 antagonists, and legumain and ZB-1 agonists). Thus, the present invention provides legumain and ZB-1 antagonists, e.g., mammalian (e.g., mouse and human) legumain and ZB-1 inhibitory polynucleotides (i.e., polynucleotides that decrease legumain and/or ZB-1 levels, secretion from cells, and/or activity, either directly or indirectly, e.g., antisense molecules, siRNA molecules, aptamers); legumain and ZB-1 inhibitory polypeptides (i.e., polypeptides that decrease legumain and/or ZB-1 levels, secretion from cells, and/or activity, either directly or indirectly, e.g., fragments of legumain or ZB-1, such as fragments containing an aberrant protease enzymatic domain, and fusion proteins thereof); antagonistic anti-legumain and ZB-1 antibodies or antibody fragments (i.e., antibodies or antibody fragments (i.e., antigen-binding fragments) that decrease legumain and/or ZB-1 levels, secretion from cells, and/or activity, either directly or indirectly, including, e.g., antagonistic antibodies and antibody fragments that bind full-length legumain and/or ZB-1 and/or fragments of legumain and/or ZB-1); and antagonistic small molecules (e.g., siRNA molecules, aptamers, and small organic molecules or compounds), which may be used to suppress legumain and/or ZB-1-mediated hydrolysis of asparaginyl bonds, and consequently, which may be used in the diagnosis, prognosis, monitoring and/or treatment of disorders related to increased legumain and/or ZB-1 activity and/or disorders treatable by decreasing legumain and/or ZB-1 activity or expression, i.e., legumain and/or ZB-1-associated conditions or disorders. As used herein the term “antagonist” refers to both direct antagonists (e.g., molecules that directly interact with legumain and/or ZB-1 polypeptides or polynucleotides) and indirect antagonists (e.g., molecules that decrease the activity, secretion, and/or expression of ZB-1 and/or legumain indirectly (e.g., RGD-containing peptides that block legumain interaction with integrins)).

The present invention also provides legumain and ZB-1 agonists, e.g., mammalian (e.g., mouse and human) legumain and ZB-1 polynucleotides (i.e., polynucleotides that increase legumain and/or ZB-1 levels, secretion from cells, and/or activity, either directly or indirectly, e.g., mRNAs, cDNAs); legumain and ZB-1 polypeptides (i.e., polypeptides that increase legumain and/or ZB-1 levels, secretion from cells, and/or activity, either directly or indirectly, e.g., fragments of legumain or ZB-1, such as fragments containing the protease enzymatic domain, and fusion proteins thereof); agonistic anti-legumain and ZB-1 antibodies or antibody fragments (i.e., antibodies or antibody fragments that increase legumain and/or ZB-1 levels, secretion from cells, and/or activity, either directly or indirectly, including, e.g., agonistic antibodies and antibody fragments that bind full-length legumain and/or ZB-1 and/or fragments of legumain and/or ZB-1); and agonistic small molecules (e.g., small organic molecules or compounds), which may be used to enhance legumain and/or ZB-1-mediated hydrolysis of asparaginyl bonds, and consequently, which may be used in the diagnosis, prognosis, monitoring and/or treatment of disorders related to decreased legumain and/or ZB-1 activity and/or disorders treatable by increasing legumain and/or ZB-1 activity or expression, e.g., disorders that are treatable by or would benefit from increased cell (e.g., endothelial cell) migration, e.g., wound healing (e.g., wounds surgically induced or occurring accidentally or otherwise), or other conditions that are treatable by or would benefit from such increased cell migration, e.g., recovery from transplant surgery. As used herein the term “agonist” refers to both direct agonists (e.g., molecules that directly interact with legumain and/or ZB-1 polypeptides or polynucleotides) and indirect agonists (e.g., molecules that increase the activity, secretion, and/or expression of ZB-1 and/or legumain indirectly (e.g., transcriptional enhancers of legumain and/or ZB-1 expression)).

As legumain is a secreted protein that is believed to interact with matrix molecules and/or cell surfaces, compounds that decrease the amount of legumain and/or ZB-1 present extracellularly are useful to modulate legumain and/or ZB-1 activity in a cell or population of cells that secrete legumain and/or ZB-1, e.g., macrophages, foam cells, vascular and endothelial cells (e.g., arterial endothelial cells), sites of inflammatory cell invasion into vessel walls (e.g., into an arterial intima), neointimal lesional areas, kidney proximal tubule cells, monocytes, etc.

Disorders related to increased legumain and/or ZB-1 activities are described herein as “legumain- and/or ZB-1-associated conditions” or “legumain- and/or ZB-1-associated disorders” or the like, and include, without limitation, atherosclerosis (including, but not limited to, all stages of atherogenesis and atherosclerosis, e.g., endothelial cell activation, formation of fatty streaks, inflammatory cell invasion of vessel walls, endothelial cell migration, formation of foam cells, plaque denudation, atheromatous plaque formation, atheromatous plaque rupture, atherothrombosis, aneurysm, stenosis, etc.), congestive heart failure, myocardial infarction, arrhythmias (e.g., atrial and ventricular arrhythmias), stenosis, aneurysm, peripheral vascular disease, chronic peripheral arterial occlusive disease (CPAOD), peripheral artery occlusive disease (PAOD), thrombosis (including, e.g., acute arterial thrombosis, atherothrombosis, and deep venous thrombosis), embolism, inflammatory vascular disorders, Raynaud's phenomenon, vasculitis and/or arteritis (including, e.g., Bechet's disease, Buerger's disease, central nervous system vasculitis, Churg-Strauss syndrome cryoglobulinemia, giant cell arteritis, Kawasaki disease, microscopic polyangitis, polyarteritis nodosa, polymyalgia rheumatica, rheumatoid vasculitis, Takayasu's arteritis, and Wegener's granulomatosis), venous disorders, hypertensive vascular disease, claudication, anginas (e.g., stable angina, unstable angina), stroke, coronary artery disease (CAD), peripheral arterial disease (PAD), acute coronary syndrome (ACS), metabolic syndrome, ischemia, reperfusion, and exacerbation of various diseases affected by the circulatory system (e.g., diabetic nephropathy, chronic kidney disease, end-stage renal disease (ESRD), hyperlipidemia, hypertension, and diabetes). Additional disorders amenable to diagnosis, prognosis, monitoring, treatment, amelioration and/or prevention using the methods disclosed herein include inflammatory disorders (e.g., chronic inflammatory disorders, including but not limited to arthritis, tuberculosis, and multiple sclerosis, as well as inflammatory bowel diseases, such as Crohn's disease and ulcerative colitis).

The present invention further provides methods of screening for: (I) legumain and/or ZB-1 antagonists, e.g., mouse and human legumain and/or ZB-1 inhibitory polynucleotides (e.g., antisense, siRNA, aptamers); legumain and/or ZB-1 inhibitory polypeptides (e.g., enzymatically inactive fragments of legumain and/or ZB-1); antagonist anti-legumain and/or -ZB-1 antibodies and antibody fragments (including antibodies and antibody fragments that bind legumain and/or ZB-1 fragments); and antagonistic small molecules (e.g., siRNAs, aptamers, and small organic molecules or compounds); and (2) compounds useful for treating, ameliorating, and/or preventing legumain- and/or ZB-1-associated disorders, e.g., vascular disorders and/or inflammatory disorders. Such screening methods may be undertaken by, e.g., measuring changes in the level of expression of legumain and/or ZB-1 (e.g., levels of legumain and/or ZB-1 mRNA, cDNA, protein and/or protein fragments), or by measuring changes in the level of activity of legumain and/or ZB-1 (e.g., changes in levels of the hydrolysis of asparaginyl bonds on substrates), or by measuring changes in the level of legumain and/or ZB-1 secretion (e.g., by using immunohistochemistry or other well known techniques).

The terms “legumain” and “ZB-1” as used herein, where appropriate, refer to mammalian legumain and ZB-1, e.g., primate (including human) and/or rodent (including mouse) legumain and ZB-1, and includes both legumain and ZB-1 polynucleotides and polypeptides.

Accordingly, the present application provides legumain and ZB-1-related polynucleotides and polypeptides. The present invention also provides antibodies and antibody fragments thereof, e.g., intact antibodies and antigen-binding fragments thereof, that bind to legumain and/or ZB-1, e.g., mammalian legumain and/or ZB-1, in particular, human, porcine and/or murine legumain and/or ZB-1. In one embodiment, an anti-legumain or ZB-1 antibody inhibits or antagonizes at least one legumain- and/or ZB-1-associated activity. For example, an anti-legumain antibody may bind legumain and inhibit (e.g., decrease, limit, neutralize, block, interfere with, or otherwise reduce) the interaction between legumain and a protein/peptide substrate. An anti-legumain antibody may also bind legumain and/or ZB-1 and interfere with legumain and/or ZB-1 enzymatic activity (e.g., protease activity, protein/peptide cleavage, protein/peptide activation) by inducing, for example, a conformational change in legumain or ZB-1 amino acid tertiary and/or secondary structure, or by preventing the processing of legumain and/or ZB-1 into a mature peptide (e.g., by preventing N-terminal or C-terminal processing of the propeptide). Thus, the antibodies of the invention may be used to detect, and optionally inhibit, a legumain and/or ZB-1 activity (e.g., interaction of legumain and/or ZB-1 with a protein/peptide substrate, or legumain and/or ZB-1 asparaginyl peptidase/protease activity). Thus, the anti-legumain antibodies and anti-ZB-1 antibodies of the invention may be used to diagnose, prognose, monitor, treat, ameliorate or prevent legumain and/or ZB-1-associated disorders, e.g., vascular and inflammatory disorders, such as atherosclerosis and arthritis.

Legumain and ZB-1 Polynucleotides and Polypeptides

The present invention provides characterization of legumain and ZB-1, e.g., expression profiles in the aorta, kidney, and atherosclerotic plaques, subcellular localization, and enzymatic activity. As such, the present invention relates to legumain and ZB-1 polynucleotides and polypeptides (e.g., full length and fragments of legumain and ZB-1 polynucleotides and polypeptides) and inhibitory legumain and ZB-1 polynucleotides and polypeptides (e.g., full length and fragments of legumain and ZB-1 inhibitory polynucleotides and polypeptides). Human legumain nucleic acid sequences, which correspond to GenBank Accession Nos. NM_{—}001008530 and NM_{—}005606, are set forth in SEQ ID NOs:1 and 3. The corresponding human legumain amino acid sequences are set forth in SEQ ID NOs:2 and 4, respectively. The mouse legumain nucleic acid sequence, which corresponds to GenBank Accession No. NM_{—}011175, is set forth in SEQ ID NO:5. The corresponding mouse legumain amino acid sequence is set forth in SEQ ID NO:6. A Pan troglodytes nucleic acid sequence predicted to be a legumain, which corresponds to GenBank Accession No. XM_{—}510133, is set forth in SEQ ID NO:7. The corresponding Pan troglodytes amino acid sequence is set forth in SEQ ID NO:8. A Macaca mulatta nucleic acid sequence predicted to be a legumain, which corresponds to GenBank Accession No. XM_{—}001092047, is set forth in SEQ ID NO:9. The corresponding Macaca amino acid sequence is set forth in SEQ ID NO:10. Additional legumain nucleotides and nucleotides predicted to be legumain proteins include those from Bos taurus (GenBank Accession No. NM_{—}174101), Ovis aries (GenBank Accession No. DQ152974), Canis familiaris (GenBank Accession Nos. XM_{—}851487 and XM_{—}537355), and Rattus norvegicus (GenBank Accession No. NM_{—}022226). “Legumain polypeptide” refers to mammalian (e.g., human and mouse) legumain proteins (including, but not limited to, allelic variants) and fragments thereof, such as the amino acid sequences set forth in SEQ ID NOs:2, 4, 6, 8 and 10. “Legumain polynucleotide” refers to mammalian (e.g., human and mouse) legumain nucleic acids (including, but not limited to, RNA and DNA, and allelic variants thereof) and fragments thereof, such as the nucleic acid sequences set forth in SEQ ID NOs:1, 3, 5, 7 and 9.

The inventors have also established that there exists high homology between legumain and ZB-1, and that ZB-1, like legumain, is a secreted protein. Thus, the present invention relates to ZB-1 polynucleotides and polypeptides (e.g., full length and active fragments of ZB-1 polynucleotides and polypeptides) and inhibitory ZB-1 polynucleotides and polypeptides (e.g., full length and fragments of ZB-1 inhibitory polynucleotides and polypeptides). The nucleotide sequence of a cDNA encoding this novel protein, designated human ZB-1, is set forth in SEQ ID NO:11. Polynucleotides of the present invention also include polynucleotides that hybridize under stringent conditions to SEQ ID NO:11, or its complement, and/or encode polypeptides that retain substantial biological activity of full-length human ZB-1. Polynucleotides of the present invention also include continuous portions of the sequence set forth in SEQ ID NO:11 comprising at least 21 consecutive nucleotides.

The deduced amino acid sequence of human ZB-1 is set forth in SEQ ID NO:12. Polypeptides of the present invention also include continuous portions of the sequence set forth in SEQ ID NO:12 comprising at least seven consecutive amino acids. A preferred polypeptide of the present invention includes any continuous portion of the sequence set forth in SEQ ID NO:12 that retains substantial biological activity (i.e., an active fragment) of human ZB-1. Polynucleotides of the present invention also include, in addition to those polynucleotides of human origin described above, polynucleotides that encode the amino acid sequence set forth in SEQ ID NO:12 or a continuous portion thereof, and that differ from the polynucleotides of human origin described above due only to the well-known degeneracy of the genetic code.

“ZB-1 polypeptide” refers to mammalian (e.g., human and mouse) ZB-1 proteins (including allelic variants) and fragments thereof, such as the amino acid sequence set forth in SEQ ID NO:12. “ZB-1 polynucleotide” refers to mammalian (e.g., human and mouse) ZB-1 nucleic acids (including RNA and DNA, and allelic variants thereof) and fragments thereof, such as the nucleic acid sequence set forth in SEQ ID NO:11. “Active fragment” refers to a portion of a legumain or ZB-1 polynucleotide or polypeptide that retains, to a substantial degree, a biological activity of a legumain or ZB-1 polynucleotide or polypeptide, e.g., asparaginyl protease/peptidase activity or encoding a polypeptide/peptide that retains asparaginyl protease/peptidase activity. One of ordinary skill in the art will know of several assays for evaluating the biological activity of such fragments.

The isolated polynucleotides of or related to the present invention may be used as hybridization probes and primers to identify and isolate nucleic acids having sequences identical to or similar to those encoding the disclosed polynucleotides. Hybridization methods for identifying and isolating nucleic acids include polymerase chain reaction (PCR), Southern hybridization, in situ hybridization and Northern hybridization, and are well known to those skilled in the art.

Hybridization reactions may be performed under conditions of different stringency. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1
Stringency Conditions

| | | Hybrid | | Wash |
|---|---|---|---|---|
| Stringency | Polynucleotide | Length | Hybridization Temperature and | Temperature and |
| Condition | Hybrid | (bp)¹ | Buffer² | Buffer² |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{F}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | <50 | T_{N}*; 6xSSC | T_{N}*; 6xSSC |
| O | DNA:RNA | >50 | 55° C.; 4xSSC -or- | 55° C.; 2xSSC |
| | | | 42° C.; 6xSSC, 50% formamide | |
| P | DNA:RNA | <50 | T_{P}*; 6xSSC | T_{P}*; 6xSSC |
| Q | RNA:RNA | >50 | 60° C.; 4xSSC -or- | 60° C.; 2xSSC |
| | | | 45° C.; 6xSSC, 50% formamide | |
| R | RNA:RNA | <50 | T_{R}*; 4xSSC | T_{R}*; 4xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ²SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete. | | | | |
| T_{B}* − T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of | | | | |
| # G + C bases). For hybrids between 18 and 49 base pairs in length, Tₘ(° C.) = 81.5 + 16.6(log₁₀Na⁺) + 0.41 (% G + C) − (600/N), where N is the number of bases in the hybrid, and Na⁺ is the concentration of sodium ions in the hybridization buffer (Na⁺ for 1xSSC = 0.165M). | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, NY, chapters 9 and 11, and Current Protocols in Molecular Biology, 1995, F. M. Ausubel et al., eds., John Wiley & Sons, Inc., sections 2.10 and 6.3-6.4, incorporated herein by reference.

The isolated polynucleotides of or related to the present invention may be used as hybridization probes and primers to identify and isolate DNA having sequences encoding allelic variants of the disclosed polynucleotides. Allelic variants are naturally occurring alternative forms of the disclosed polynucleotides that encode polypeptides that are identical to or have significant similarity to the polypeptides encoded by the disclosed polynucleotides. Preferably, allelic variants have at least 90% sequence identity (more preferably, at least 95% identity; most preferably, at least 99% identity) with the disclosed polynucleotides. Alternatively, significant similarity exists when the nucleic acid segments will hybridize under selective hybridization conditions (e.g., highly stringent hybridization conditions) to the disclosed polynucleotides. Such variants are encompassed within the scope of the present invention.

The isolated polynucleotides of or related to the present invention may also be used as hybridization probes and primers to identify and isolate DNAs having sequences encoding polypeptides homologous to the disclosed polynucleotides. These homologs are polynucleotides and polypeptides isolated from a different species than that of the disclosed polypeptides and polynucleotides, or within the same species, but with significant sequence similarity to the disclosed polynucleotides and polypeptides. Preferably, polynucleotide homologs have a high sequence identity, e.g., at least 50% sequence identity (more preferably, at least 75% identity, e.g., 80%, or 85% identity; most preferably, at least 90% identity, e.g., 92%, 94%, 96%, 98%, or 99% identity) with the disclosed polynucleotides, whereas polypeptide homologs have a high sequence identity, e.g., at least 30% sequence identity (more preferably, at least 45% identity, e.g., 50%, or 55% identity; most preferably, at least 60% identity, e.g., 65%, 70%, 75%, 80%, 85%, 90%, 95%, or 99% identity) with the disclosed polypeptides. Preferably, homologs of the disclosed polynucleotides and polypeptides are those isolated from mammalian species. Such homologs are encompassed within the scope of the present invention.

Calculations of “homology” or “sequence identity” between two sequences may be performed by comparison methods well known in the art. For example, regarding identity, the sequences are aligned for optimal comparison purposes (e.g., gaps can be introduced in one or both of a first and a second amino acid or nucleic acid sequence for optimal alignment, and nonhomologous sequences can be disregarded for comparison purposes). In one embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent sequence identity between two sequences may be accomplished using a mathematical algorithm. In one embodiment, the percent identity between two amino acid sequences is determined using the Needleman and Wunsch ((1970) J. Mol. Biol. 48:444-53) algorithm, which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In another embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A preferred set of parameters (and the one that should be used if the practitioner is uncertain about what parameters should be applied to determine whether a molecule is within a sequence identity or homology limitation of the invention) is a Blossum 62 scoring matrix with a gap penalty of 12, a gap extend penalty of 4, and a frameshift gap penalty of 5. The percent identity between two amino acid or nucleotide sequences can also be determined using the algorithm of Meyers and Miller ((1989) CABIOS 4:11-17), which has been incorporated into the ALIGN program (version 2.0), using a PAM120 weight residue table, a gap length penalty of 12 and a gap penalty of 4.

The isolated polynucleotides of or related to the present invention may also be used as hybridization probes and primers to identify cells and tissues that express the polypeptides of or related to the present invention and the conditions under which they are expressed.

Additionally, the function of the polypeptides of or related to the present invention may be directly examined by using the polynucleotides encoding the polypeptides to alter (i.e., enhance, reduce, or modify) the expression of the genes corresponding to the polynucleotides of or related to the present invention in a cell or organism. These “corresponding genes” are the genomic DNA sequences of or related to the present invention that are transcribed to produce the mRNAs from which the polynucleotides of or related to the present invention are derived.

Altered expression of the genes of or related to the present invention may be achieved in a cell or organism through the use of various inhibitory polynucleotides, such as antisense polynucleotides, siRNAs, and ribozymes that bind and/or cleave the mRNA transcribed from the genes of or related to the invention (see, e.g., Galderisi et al. (1999) J. Cell Physiol. 181:251-57; Sioud (2001) Curr. Mol. Med. 1:575-88). Inhibitory polynucleotides to legumain and/or ZB-1 may be useful asparaginyl peptidase antagonists and, as such, may also be useful in treating, ameliorating and/or preventing legumain and/or ZB-1-associated disorders, e.g., vascular and inflammatory disorders (e.g., atherosclerosis and arthritis). Inhibitory polynucleotides may also consist of aptamers, i.e., polynucleotides that bind to and regulate protein activity, e.g., the activity of human legumain and/or ZB-1. Aptamers are described throughout the literature (see, e.g., Nimjee et al. (2005) Annu. Rev. Med. 56:555-83; Patel (1997) Curr. Opin. Chem. Biol. 1:32-46; Pendergrast et al. (2005) J. Biomol. Tech. 16:224-34; Proske et al. (2005) Appl. Microbiol. Biotechnol. 69:367-74; Blank and Blind (2005) Curr. Opin. Chem. Biol. 9:336-42; Tombelli et al. (2005) Biosens. Bioelectron. 20(12):2424-34; and Di Gusto et al. (2006) Chembiochem. 7(3):535-44).

The antisense polynucleotides or ribozymes related to the invention may be complementary to an entire coding strand of a gene of or related to the invention, or to only a portion thereof. Alternatively, antisense polynucleotides or ribozymes can be complementary to a noncoding region of the coding strand of a gene of or related to the invention. The antisense polynucleotides or ribozymes can be constructed using chemical synthesis and enzymatic ligation reactions using procedures well known in the art. The nucleoside linkages of chemically synthesized polynucleotides can be modified to enhance their ability to resist nuclease-mediated degradation, as well as to increase their sequence specificity. Such linkage modifications include, but are not limited to, phosphorothioate, methylphosphonate, phosphoroamidate, boranophosphate, morpholino, and peptide nucleic acid (PNA) linkages (Galderisi et al., supra; Heasman (2002) Dev. Biol. 243:209-14; Micklefield (2001) Curr. Med. Chem. 8:1157-79). Alternatively, these molecules can be produced biologically using an expression vector into which a polynucleotide of or related to the present invention has been subcloned in an antisense (i.e., reverse) orientation.

The inhibitory polynucleotides of the present invention also include triplex-forming oligonucleotides (TFOs) that bind in the major groove of duplex DNA with high specificity and affinity (Knauert and Glazer (2001) Hum. Mol. Genet. 10:2243-51). Expression of the genes of or related to the present invention can be inhibited by targeting TFOs complementary to the regulatory regions of the genes (i.e., the promoter and/or enhancer sequences) to form triple helical structures that prevent transcription of the genes.

In one embodiment of the invention, the inhibitory polynucleotides of the present invention are short interfering RNA (siRNA) molecules. These siRNA molecules are short (preferably 19-25 nucleotides; most preferably 19 or 21 nucleotides), double-stranded RNA molecules that cause sequence-specific degradation of target mRNA. This degradation is known as RNA interference (RNAi) (e.g., Bass (2001) Nature 411:428-29). Originally identified in lower organisms, RNAi has been effectively applied to mammalian cells and has recently been shown to prevent fulminant hepatitis in mice treated with siRNA molecules targeted to Fas mRNA (Song et al. (2003) Nature Med. 9:347-51). In addition, intrathecally delivered siRNA has recently been reported to block pain responses in two models (agonist-induced pain model and neuropathic pain model) in the rat (Dorn et al. (2004) Nucleic Acids Res. 32(5):e49).

The siRNA molecules of the present invention may be generated by annealing two complementary single-stranded RNA molecules together (one of which matches a portion of the target mRNA) (Fire et al., U.S. Pat. No. 6,506,559) or through the use of a single hairpin RNA molecule that folds back on itself to produce the requisite double-stranded portion (Yu et al. (2002) Proc. Natl. Acad. Sci. USA 99:6047-52). The siRNA molecules may be chemically synthesized (Elbashir et al. (2001) Nature 411:494-98) or produced by in vitro transcription using single-stranded DNA templates (Yu et al., supra). Alternatively, the siRNA molecules can be produced biologically, either transiently (Yu et al., supra; Sui et al. (2002) Proc. Natl. Acad. Sci. USA 99:5515-20) or stably (Paddison et al. (2002) Proc. Natl. Acad. Sci. USA 99:1443-48; Cullen (2006) Gene Therapy 13:503-08), using an expression vector(s) containing the sense and antisense siRNA sequences. Recently, reduction of levels of target mRNA in primary human cells, in an efficient and sequence-specific manner, was demonstrated using adenoviral vectors that express hairpin RNAs, which are further processed into siRNAs (Arts et al. (2003) Genome Res. 13:2325-32).

The siRNA molecules targeted to the polynucleotides of or related to the present invention can be designed based on criteria well known in the art (e.g., Elbashir et al. (2001) EMBO J. 20:6877-88; Aronin (2006) Gene Therapy 13:509-16). For example, the target segment of the target mRNA preferably should begin with AA (most preferred), TA, GA, or CA; the GC ratio of the siRNA molecule preferably should be 45-55%; the siRNA molecule preferably should not contain three of the same nucleotides in a row; the siRNA molecule preferably should not contain seven mixed G/Cs in a row; and the target segment preferably should be in the ORF region of the target mRNA and preferably should be at least 75 bp after the initiation ATG and at least 75 bp before the stop codon. Based on these criteria, or on other known criteria (e.g., Reynolds et al. (2004) Nature Biotechnol. 22:326-30), siRNA molecules related to the present invention that target the mRNA polynucleotides of or related to the present invention may be designed by one of skill in the art.

Altered expression of the genes of or related to the present invention in an organism may also be achieved through the creation of nonhuman transgenic animals into whose genomes polynucleotides of or related to the present invention have been introduced. Such transgenic animals include animals that have multiple copies of a gene (i.e., the transgene) of the present invention. A tissue-specific regulatory sequence(s) may be operably linked to the transgene to direct expression of a polypeptide of or related to the present invention to particular cells or a particular developmental stage. Methods for generating transgenic animals via embryo manipulation and microinjection, particularly animals such as mice, have become conventional and are well known in the art (e.g., Bockamp et al. (2002) Physiol. Genomics 11:115-32).

Altered expression of the genes of or related to the present invention in an organism may also be achieved through the creation of animals whose endogenous genes corresponding to the polynucleotides of or related to the present invention have been disrupted through insertion of extraneous polynucleotide sequences (i.e., a knockout animal). The coding region of the endogenous gene may be disrupted, thereby generating a nonfunctional protein. Alternatively, the upstream regulatory region of the endogenous gene may be disrupted or replaced with different regulatory elements, resulting in the altered expression of the still-functional protein. Methods for generating knockout animals include homologous recombination and are well known in the art (e.g., Wolfer et al. (2002) Trends Neurosci. 25:336-40).

The isolated polynucleotides of or related to the present invention also may be operably linked to an expression control sequence and/or ligated into an expression vector for recombinant production of the polypeptides (including active fragments and/or fusion polypeptides thereof) of or related to the present invention. General methods of expressing recombinant proteins are well known in the art.

An expression vector, as used herein, is intended to refer to a nucleic acid molecule capable of transporting another nucleic acid to which it has been linked. One type of vector is a plasmid, which refers to a circular double-stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., nonepisomal mammalian vectors) can be integrated into the genome of a host cell upon introduction into the host cell, and thereby are replicated along with the host genome. Moreover, certain vectors are capable of directing the expression of genes to which they are operably linked. Such vectors are referred to herein as recombinant expression vectors (or simply, expression vectors). In general, expression vectors of utility in recombinant DNA techniques are often in the form of plasmids. In the present specification, plasmid and vector may be used interchangeably, as the plasmid is the most commonly used form of vector. However, the invention is intended to include other forms of expression vectors, such as viral vectors (e.g., replication-defective retroviruses, adenoviruses and adeno-associated viruses) that serve equivalent functions.

In one embodiment, the polynucleotides of or related to the present invention are used to create recombinant legumain and/or ZB-1 antagonists. An example of a legumain antagonist includes enzymatically inactive legumain (polypeptide or polynucleotide) and enzymatically inactive fragments thereof. An example of a ZB-1 antagonist includes enzymatically inactive ZB-1 (polypeptide or polynucleotide) and enzymatically inactive fragments thereof. Enzymatically inactive legumain and/or ZB-1 include molecules that contain all or part of the N-terminal and/or C-terminal propeptides (e.g., the sequence N-terminal to amino acid position 21 or 25 and/or the sequence C-terminal to amino acid position 323). Such antagonists may be useful in regulating asparaginyl protease activity, and consequently, in the treatment of atherosclerosis or other vascular and inflammatory disorders in which it is desirable to decrease asparaginyl bond hydrolysis. In another embodiment, the polynucleotides of or related to the present invention are used to create other legumain and ZB-1 antagonists, e.g., legumain and/or ZB-1 inhibitory polynucleotides, legumain and/or ZB-1 inhibitory polypeptides (including fragments and fusion proteins thereof), antagonistic anti-legumain antibodies and fragment thereof, antagonistic anti-ZB-1 antibodies and fragments thereof, and antagonistic small molecules.

Methods of creating fusion polypeptides, i.e., a first polypeptide moiety linked with a second polypeptide moiety, are well known in the art. For example, a legumain and/or ZB-1 polypeptide may be fused to a second polypeptide moiety, e.g., an immunoglobulin or a fragment thereof (e.g., an Fe fragment). In some embodiments, the first polypeptide moiety includes, e.g., a full-length human legumain or ZB-1 polypeptide. Alternatively, the first polypeptide may comprise less than the full-length legumain or ZB-1 polypeptide (e.g., a substrate binding domain of legumain or ZB-1). Additionally, soluble forms of legumain or ZB-1 may be fused through “linker” sequences to the Fe portion of an immunoglobulin. Other fusions proteins, such as those with glutathione-S-transferase (GST), Lex-A, thioredoxin (TRX) or maltose-binding protein (MBP), may also be used.

The second polypeptide moiety is preferably soluble. In some embodiments, the second polypeptide moiety enhances the half-life, (e.g., the serum half-life) of the linked polypeptide. In some embodiments, the second polypeptide moiety includes a sequence that facilitates association of the fusion polypeptide with a legumain or ZB-1 polypeptide. In one embodiment, the second polypeptide includes at least a region of an immunoglobulin polypeptide. Immunoglobulin fusion polypeptides are known in the art and are described in, e.g., U.S. Pat. Nos. 5,516,964; 5,225,538; 5,428,130; 5,514,582; 5,714,147; and 5,455,165, all of which are hereby incorporated by reference herein in their entireties. The fusion proteins may additionally include a linker sequence joining the first polypeptide moiety, e.g., human legumain or ZB-1, including fragments thereof, to the second moiety. Use of such linker sequences are well known in the art. For example, the fusion protein can include a peptide linker, e.g., a peptide linker of about 2 to 20, more preferably less than 10, amino acids in length. In one embodiment, the peptide linker may be 2 amino acids in length. In other embodiments, a fusion protein of or related to the invention includes more than two polypeptide moieties, e.g., a tripartite fusion protein may comprise two legumain polypeptides (or fragments thereof), two ZB-1 polypeptides (or fragments thereof), or a combination thereof linked by a third polypeptide moiety that facilitates association of the two legumain polypeptides (or fragments thereof), two ZB-1 polypeptides (or fragments thereof), or a combination thereof.

In another embodiment, the recombinant protein includes a heterologous signal sequence (i.e., a polypeptide sequence that is not present in a polypeptide encoded by a legumain or ZB-1 polynucleotide) at its N-terminus. For example, a signal sequence from another protein may be fused with a legumain or ZB-1 polypeptide, including fragments and/or fusion proteins thereof. In certain host cells (e.g., mammalian host cells), expression and/or secretion of recombinant proteins can be increased through use of a heterologous signal sequence. A signal peptide that may be included in the fusion protein is the melittin signal peptide with the sequence: MKFLVNVALVFMVVYISYIYA (SEQ ID NO:13).

A fusion protein of the invention may be produced by standard recombinant DNA techniques. For example, DNA fragments coding for the different polypeptide sequences are ligated together in-frame in accordance with conventional techniques by employing, e.g., blunt-ended or sticky-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments may be carried out using anchor primers that give rise to complementary overhangs between two consecutive gene fragments that can subsequently be annealed and reamplified to generate a chimeric gene sequence (see, for example, Current Protocols in Molecular Biology, Ausubel et al. (eds.), John Wiley & Sons, 1992). Moreover, many expression vectors are commercially available that encode a fusion moiety (e.g., an Fc region of an immunoglobulin heavy chain). A legumain-encoding or a ZB-1-encoding nucleic acid may be cloned into such an expression vector such that the fusion moiety is linked in-frame to the immunoglobulin protein.

The recombinant expression vectors of the invention may carry additional sequences, such as sequences that regulate replication of the vector in host cells (e.g., origins of replication) and selectable marker genes. The selectable marker gene facilitates selection of host cells into which the vector has been introduced. For example, typically the selectable marker gene confers resistance to drugs, such as G418, hygromycin or methotrexate, to a host cell into which the vector has been introduced. Preferred selectable marker genes include the dihydrofolate reductase (DHFR) gene (for use in dhfr⁻ host cells with methotrexate selection/amplification) and the neo gene (for G418 selection).

Suitable vectors can be chosen or constructed, containing appropriate regulatory sequences, including promoter sequences, terminator sequences, polyadenylation sequences, enhancer sequences, marker genes and other sequences, e.g., sequences that regulate replication of the vector in the host cells (e.g., origins of replication) as appropriate. Vectors may be plasmids or viral, e.g., phage, or phagemid, as appropriate. For further details see, for example, Molecular Cloning: a Laboratory Manual: 2nd ed., Sambrook et al., Cold Spring Harbor Laboratory Press, 1989. Many known techniques and protocols for manipulation of nucleic acids, for example, in preparation of nucleic acid constructs, mutagenesis, sequencing, introduction of DNA into cells and gene expression, and analysis of proteins, are described in detail in Current Protocols in Molecular Biology, 2nd ed., Ausubel et al. (eds.) John Wiley & Sons, 1992.

A further aspect of the present invention provides a host cell comprising a nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection, and transduction using retrovirus or other viruses, e.g., vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g., by culturing host cells under conditions for expression of the gene.

A number of cell lines may act as suitable host cells for recombinant expression of the polypeptides of or related to the present invention. Mammalian host cell lines include, for example, COS cells, CHO cells, 3T3-L1, 293 cells, A431 cells, 3T3 cells, CV-1 cells, HeLa cells, L cells, BHK21 cells, HL-60 cells, U937 cells, HaK cells, Jurkat cells, THP-1 cells as well as cell strains derived from in vitro culture of primary tissue and primary explants.

Alternatively, it may be possible to recombinantly produce the polypeptides of or related to the present invention in lower eukaryotes, such as yeast, or in prokaryotes. Potentially suitable yeast strains include Saccharomyces cerevisiae, Schizosaccharomyces pombe, Kluyveromyces strains, and Candida strains. Potentially suitable bacterial strains include Escherichia coli, Bacillus subtilis, and Salmonella typhimurium. If the polypeptides of or related to the present invention are made in yeast or bacteria, it may be necessary to modify them by, for example, phosphorylation or glycosylation of appropriate sites, in order to obtain functionality. Such covalent attachments may be accomplished using well-known chemical or enzymatic methods.

Expression in bacteria may result in formation of inclusion bodies incorporating the recombinant protein. Thus, refolding of the recombinant protein may be required in order to produce active or more active material. Several methods for obtaining correctly folded heterologous proteins from bacterial inclusion bodies are known in the art. These methods generally involve solubilizing the protein from the inclusion bodies, then denaturing the protein completely using a chaotropic agent. When cysteine residues are present in the primary amino acid sequence of the protein, it is often necessary to accomplish the refolding in an environment that allows correct formation of disulfide bonds (a redox system). General methods of refolding are disclosed in, e.g., Kohno (1990) Meth. Enzymol. 185:187-95. Other appropriate methods are disclosed in, e.g., EP 0433225 and U.S. Pat. No. 5,399,677.

The polypeptides of or related to the present invention may also be recombinantly produced by operably linking the isolated polynucleotides of the present invention to suitable control sequences in one or more insect expression vectors, such as baculovirus vectors, and employing an insect cell expression system. Materials and methods for baculovirus/Sf9 expression systems are commercially available in kit form (e.g., BAC-TO-BAC® and MAXBAC® kits, Invitrogen, Carlsbad, Calif.).

Following recombinant expression in the appropriate host cells, the recombinant polypeptides of the present invention may then be purified from cell extracts using known purification processes, such as immunoprecipitation, gel filtration and ion exchange chromatography. For example, membrane-bound forms of a legumain and/or ZB-1 polypeptide may be purified by preparing a total membrane fraction from the expressing cell and extracting the membranes with a nonionic detergent such as Triton X-100. A polypeptide of or related to the present invention may be concentrated using a commercially available protein concentration filter, for example, an AMICON® or Millipore PELLICON® ultrafiltration unit (Millipore, Billerica, Mass.). Following the concentration step, the concentrate can be applied to a purification matrix such as a gel filtration medium. Alternatively, an anion exchange resin can be employed, for example, a matrix or substrate having pendant diethylaminoethyl (DEAE) or polyetheyleneimine (PEI) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred (e.g., S-SEPHAROSE® columns, Sigma-Aldrich, St. Louis, Mo.). The purification of recombinant proteins from culture supernatant may also include one or more column steps over such affinity resins as concanavalin A-agarose, heparin-TOYOPEARL® (Toyo Soda Manufacturing Co., Ltd., Japan) or Cibacrom blue 3GA SEPHAROSE® (Tosoh Biosciences, San Francisco, Calif.); or by hydrophobic interaction chromatography using such resins as phenyl ether, butyl ether, or propyl ether; or by immunoaffinity chromatography. Finally, one or more reverse-phase high performance liquid chromatography (RP-HPLC) steps employing hydrophobic RP-HPLC media, e.g., silica gel having pendant methyl or other aliphatic groups, can be employed to further purify the recombinant protein. Affinity columns including antibodies (e.g., those described using the methods herein) to the recombinant protein may also be used in purification in accordance with known methods. Some or all of the foregoing purification steps, in various combinations or with other known methods, may also be employed to provide a substantially purified isolated recombinant protein. Preferably, the isolated recombinant protein is purified so that it is substantially free of other mammalian proteins. Additionally, these purification processes may also be used to purify the polypeptides of the present invention from other sources, including natural sources. For example, polypeptides of or related to the invention, e.g., mouse and human legumain and ZB-1 (e.g., full length or fragments of legumain or ZB-1, and fusions thereof), which are expressed as a product of transgenic animals, e.g., as a component of the milk of transgenic animals, e.g., transgenic cows, goats, pigs, or sheep, may be purified as described above.

Alternatively, the polypeptides may also be recombinantly expressed in a form that facilitates purification. For example, the polypeptides may be expressed as fusions with proteins such as maltose-binding protein (MBP), glutathione-S-transferase (GST), or thioredoxin (TRX). Kits for expression and purification of such fusion proteins are commercially available from New England BioLabs (Beverly, Mass.), Pharmacia (Piscataway, N.J.), and Invitrogen, respectively. Recombinant proteins can also be tagged with a small epitope and subsequently identified or purified using a specific antibody to the epitope. A preferred epitope is the FLAG epitope, which is commercially available from Eastman Kodak (New Haven, Conn.).

The polypeptides of or related to the present invention, including legumain and/or ZB-1 antagonists, may also be produced by known conventional chemical synthesis. Methods for chemically synthesizing such polypeptides are well known to those skilled in the art. Such chemically synthetic polypeptides may possess biological properties in common with the natural, purified polypeptides, and thus may be employed as biologically active or immunological substitutes for the natural polypeptides.

The polypeptides of or related to the present invention, including legumain and/or ZB-1 antagonists, also encompass molecules that are structurally different from the disclosed polypeptides (e.g., which have a slightly altered sequence), but have substantially the same biochemical properties as the disclosed polypeptides (e.g., are changed only in functionally nonessential amino acid residues). Such molecules include naturally occurring allelic variants and deliberately engineered variants containing alterations, substitutions, replacements, insertions, or deletions. Techniques for such alterations, substitutions, replacements, insertions, or deletions are well known to those skilled in the art. In some embodiments, the polypeptide moiety is provided as a variant polypeptide having mutations in the naturally occurring sequence (wild type) that results in a sequence more resistant to proteolysis (relative to the nonmutated sequence).

Some amino acid sequences of legumain and ZB-1 can be varied without significantly modifying legumain or ZB-1 structure or function. To retain a particular structure or function, it is possible to replace residues that form legumain or ZB-1 protein tertiary structure, provided that the substituting residue performs a similar function. In other instances, the type of residue may be completely irrelevant if an alteration occurs in a noncritical area. Thus, the invention further includes legumain or ZB-1 variants. Such variants include deletions, insertions, inversions, repeats, and type substitutions (for example, substituting one hydrophilic residue for another, but not a strongly hydrophilic residue for a strongly hydrophobic residue). Small changes or “neutral” amino acid substitutions will often have little impact on protein function (Taylor (1986) J. Theor. Biol. 119:205-18). Conservative substitutions may include, but are not limited to, replacements among the aliphatic amino acids, exchange of acidic residues, substitution between amide residues, exchange of basic residues, and replacements among the aromatic residues. Further guidance concerning what amino acid change is likely to be phenotypically silent or noisy can be found in Bowie et al. ((1990) Science 247:1306-10) and Zvelebil et al. ((1987) J. Mol. Biol. 195:957-61). Thus, legumain and/or ZB-1 polynucleotides and polypeptides may be naturally occurring or may be produced by altering various residues without changing substrate specificity and enzymatic activity. Alternatively, one of skill in the art would be able to produce legumain and/or ZB-1 polynucleotides and polypeptides with altered substrate specificity and enzymatic activity using the disclosed polynucleotide and polypeptide sequences.

Mammalian legumain is highly conserved and contains the pfam01650.12 “Peptidase_C13” conserved domain, which may be used as a guide to design and construct recombinant legumain and ZB-1 polynucleotides and polypeptides that display different substrate specificity, substrate affinity, and enzymatic activity. For example, Chen et al. ((2000) supra) report that an inactive legumain may be produced by mutating the active site cysteine, i.e., residue Cys(189). In addition, mammalian legumain undergoes both C- and N-terminal processing of propeptides to induce activation (id.; Li et al., supra). It has been shown that an inactive legumain may be produced by simply replacing the C-terminal cleavage site, i.e., Asn(323), with various residues such as aspartate, serine, alanine, or glutamate (Chen et al. (2000) supra). Additionally, legumain activity may be reduced by mutating the N-terminal cleavage sites, i.e., Asp(21) or Asp(25), e.g., via alanine replacement (Li et al., supra). Thus, as used herein “legumain” and “ZB-1” additionally refer to these and other derivative and variant polypeptide and polynucleotide sequences. Such derivatives and variants are deemed to be within the scope and knowledge of those skilled in the art.

Legumain and/or ZB-1 polypeptides, fragments and/or fusion polypeptides thereof, recombinant and/or natural forms thereof, variant and/or naturally occurring forms thereof, may be used to screen for agents (e.g., other legumain and/or ZB-1 antagonists, e.g., anti-legumain antibodies) that are capable of binding legumain and/or ZB-1 and/or regulating legumain and/or ZB-1 activity, as described further herein. Binding assays utilizing a desired binding protein, immobilized or not, are well known in the art and may be used for this purpose with the polypeptides of or related to the present invention, including the legumain and/or ZB-1 antagonists of the invention, e.g., legumain polynucleotides and polypeptides. Purified cell-based or protein-based (cell-free) screening assays may be used to identify such agents. For example, legumain and/or ZB-1 polypeptides may be immobilized in purified form on a carrier, and binding of potential substrates/ligands/antagonists to purified legumain and/or ZB-1 may be measured.

Antibodies

In other embodiments, the invention provides legumain and/or ZB-1 antagonists as antibodies and antibody fragments thereof, i.e., intact antibodies and antigen-binding fragments thereof, that specifically bind to legumain and/or ZB-1 and/or fragments thereof, preferably mammalian (e.g., human or mouse) legumain and/or ZB-1. In one embodiment, the antibodies are inhibitory antibodies, i.e., they inhibit or reduce at least one legumain and/or ZB-1 activity (e.g., hydrolysis of asparaginyl bonds) and may be useful in diagnosing, prognosing, monitoring, treating, ameliorating and/or preventing legumain- and/or ZB-1-associated disorders, e.g., vascular and/or inflammatory disorders. Additionally, the invention provides anti-legumain antibodies and anti-ZB-1 antibodies that specifically bind to legumain and/or ZB-1 (respectively or concurrently, as appropriate throughout), but do not inhibit legumain and/or ZB-1 activity (i.e., detecting antibodies); such antibodies may be used to detect the presence of, e.g., legumain or ZB-1 protein, e.g., as part of a kit for diagnosing, prognosing, and/or monitoring legumain- and/or ZB-1-associated disorders, e.g., vascular and/or inflammatory disorders. In one embodiment, the antibody is directed to legumain, preferably mammalian legumain, more preferably human legumain. In another embodiment, the antibody is directed to ZB-1, preferably mammalian ZB-1, more preferably human ZB-1. In another embodiment, the antibody is a monoclonal or single specificity antibody. The antibodies may also be human, humanized, chimeric, or in vitro-generated antibodies against, e.g., human or mouse legumain and/or human or mouse ZB-1.

One of skill in the art will recognize that, as used herein, the term “antibody” refers to a protein comprising at least one, and preferably two, heavy (H) chain variable regions (abbreviated herein as VH), and at least one and preferably two light (L) chain variable regions (abbreviated herein as VL). The antibody may further include a heavy and light chain constant region to thereby form a heavy and light immunoglobulin chain, respectively. In one embodiment, the antibody is a tetramer of two heavy immunoglobulin chains and two light immunoglobulin chains, wherein the heavy and light immunoglobulin chains are interconnected, e.g., by disulfide bonds.

The “antigen binding fragment,” e.g., of an antibody (or simply “antibody portion,” or “fragment”), as used herein, refers to one or more fragments, e.g., of a full-length antibody, that retain the ability to specifically bind to an antigen. Examples of binding fragments encompassed within the term “antigen binding fragment” of an antibody include, but are not limited to: (i) an Fab fragment, a monovalent fragment consisting of the VL, VH, CL and CH1 domains; (ii) an F(ab′)₂ fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody; (v) a dAb fragment, which consists of a VH domain; and (vi) an isolated complementarity determining region (CDR). Furthermore, although the two domains of the Fv fragment, VL and VH, are encoded by separate genes, they may be joined, using recombinant methods, by a synthetic linker that enables their production as a single protein chain in which the VL and VH regions pair to form monovalent molecules (known as single chain Fv (scFv)). Such single chain antibodies are also intended to be encompassed within the term “antigen binding fragment.” These antibody fragments, or antigen binding fragments, are obtained using conventional techniques known to those skilled in the art, and the fragments are screened for utility in the same manner as are intact antibodies.

In some embodiments, the invention provides single domain antibodies. Single domain antibodies can include antibodies whose CDRs are part of a single domain polypeptide. Examples include, but are not limited to, heavy chain antibodies, antibodies naturally devoid of light chains, single domain antibodies derived from conventional four-chain antibodies, engineered antibodies and single domain scaffolds other than those derived from antibodies. Single domain antibodies may be any of those known in the art, or any future single domain antibodies. Single domain antibodies may be derived from any species including, but not limited to, mouse, human, camel, llama, goat, rabbit, bovine. According to one aspect of the invention, a single domain antibody as used herein is a naturally occurring single domain antibody known as heavy chain antibody devoid of light chains. Such single domain antibodies are disclosed in, e.g., WO 94/04678. This variable domain derived from a heavy chain antibody naturally devoid of light chain is known herein as a VHH or nanobody, to distinguish it from the conventional VH of four-chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHH molecules are within the scope of the invention.

Antibody molecules to the polypeptides of or related to the present invention, e.g., antibodies to legumain and/or ZB-1, may be produced by methods well known to those skilled in the art. Legumain and/or ZB-1 proteins of the invention may also be used to immunize animals to obtain polyclonal and monoclonal antibodies that react with the legumain and/or ZB-1 protein and which may inhibit the interaction of a substrate with legumain and/or ZB-1. A full-length polypeptide of the present invention may be used as the immunogen, or, alternatively, antigenic peptide fragments of the polypeptides may be used. An antigenic peptide of a polypeptide of the present invention comprises at least 7 continuous amino acid residues and encompasses an epitope such that an antibody raised against the peptide forms a specific immune complex with the polypeptide. Preferably, the antigenic peptide comprises at least 10 amino acid residues, more preferably at least 15 amino acid residues, even more preferably at least 20 amino acid residues, and most preferably at least 30 amino acid residues.

In a further improvement on the procedure for producing antibodies, a method for identifying a clinically relevant epitope on an immunogen, and a correlative method for selecting an antibody that binds immunospecifically to the relevant epitope with high affinity, are disclosed in, e.g., U.S. Published Patent Application No. 2002/0029391, which is hereby incorporated by reference herein in its entirety. Exemplary epitopes generally useful for targeting lipid asparaginyl peptidases and other cysteine peptidases are discussed in Coleman and Lee (2004) supra.

Monoclonal antibodies may be generated by other methods known to those skilled in the art of recombinant DNA technology. As an alternative to preparing monoclonal antibody-secreting hybridomas, a monoclonal antibody to a polypeptide of the present invention may be identified and isolated by screening a recombinant combinatorial immunoglobulin library (e.g., an antibody phage display library) with a polypeptide of or related to the present invention (e.g., mouse and human legumain and/or ZB-1 and fragments thereof) to thereby isolate immunoglobulin library members that bind to the polypeptides of or related to the present invention. The “combinatorial antibody display” method is well known and was developed to identify and isolate antibody fragments having a particular antigen specificity, and can be utilized to produce monoclonal antibodies.

Polyclonal sera and antibodies may be produced by immunizing a suitable subject with a polypeptide of or related to the present invention. The antibody titer in the immunized subject may be monitored over time, and the antibody molecules directed against a polypeptide of the present invention may be isolated from the subject or culture media and further purified by well-known techniques.

Fragments of antibodies to the polypeptides of the present invention may be produced by cleavage of the antibodies in accordance with methods well known in the art. For example, immunologically active Fab and F(ab′)₂ fragments may be generated by treating the antibodies with an enzyme such as pepsin.

Additionally, chimeric, humanized, and single-chain antibodies to the polypeptides of the present invention, comprising both human and nonhuman portions, may be produced using standard recombinant DNA techniques and/or a recombinant combinatorial immunoglobulin library. For example, human monoclonal antibodies (mAbs) directed against, e.g., human legumain and/or ZB-1, may be generated using transgenic mice carrying the human immunoglobulin genes rather than murine immunoglobulin genes.

Monoclonal, chimeric, human and humanized antibodies that have been modified by, e.g., deleting, adding, or substituting other portions of the antibody, e.g., the constant region, are also within the scope of the invention. As nonlimiting examples, an antibody can be modified by deleting the constant region, by replacing the constant region with another constant region, e.g., a constant region meant to increase half-life, stability, or affinity of the antibody, or a constant region from another species or antibody class, and by modifying one or more amino acids in the constant region to alter, for example, the number of glycosylation sites, effector cell function, Fc receptor (FcR) binding, complement fixation, etc.

Antibodies with altered function, e.g., altered affinity for an effector ligand, such as FcR on a cell, or the C1 component of complement, can be produced by replacing at least one amino acid residue in the constant portion of the antibody with a different residue (see, e.g., EP 388,151, U.S. Pat. Nos. 5,624,821 and 5,648,260, the contents of all of which are hereby incorporated by reference herein in their entireties).

In addition to antibodies for use in the instant invention, other molecules may also be employed to modulate the activity of legumain and/or ZB-1. Such molecules include small modular immunopharmaceutical (SMIP™) drugs (Trubion Pharmaceuticals, Seattle, Wash.). SMIPs are single-chain polypeptides composed of a binding domain for a cognate structure such as an antigen, a counter receptor or the like, a hinge-region polypeptide having either one or no cysteine residues, and immunoglobulin CH2 and CH3 domains (see also www.trubion.com). SMIPs and their uses and applications are disclosed in, e.g., U.S. Published Patent Appln. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

Anti-legumain antibodies and/or anti-ZB-1 antibodies of the invention may be useful for isolating, purifying, and/or detecting legumain and/or ZB-1 polypeptides and legumain and/or ZB-1 polypeptide fragments (or fusions thereof), in supernatant, in cellular lysate, on a cell surface, or within the extracellular matrix. Antibodies disclosed in this invention also may be used diagnostically to monitor, e.g., legumain and/or ZB-1 polypeptide levels, as part of a clinical testing procedure, or clinically to target a therapeutic modulator to a cell or tissue comprising the antigen of the antibody. For example, a therapeutic, such as a small molecule or other therapeutic of the invention, may be linked to an anti-legumain antibody and/or an anti-ZB-1 antibody in order to target the therapeutic to the cell or tissue expressing legumain and/or ZB-1. Antagonistic antibodies (including, but not limited to, monoclonal antibodies) that bind to legumain and/or ZB-1 polypeptides may also be useful in the treatment of a disease(s) related to legumain and/or ZB-1 activity, or legumain- and/or ZB-1-associated conditions. Thus, the present invention further provides compositions comprising an inhibitory (antagonist) antibody that specifically binds to legumain and/or ZB-1 and which decreases, limits, blocks, or otherwise reduces legumain and/or ZB-1 activity. Similarly, anti-legumain and/or anti-ZB-1 antibodies may be useful in isolating, purifying, detecting, and/or diagnostically monitoring legumain and/or ZB-1, and/or clinically targeting a therapeutic modulator to a cell or tissue comprising legumain and/or ZB-1.

Screening Assays

The legumain and ZB-1 polynucleotides and polypeptides may be used in screening assays to identify pharmacological agents or lead compounds for agents that are capable of modulating the activity of legumain and/or ZB-1 in a cell or organism, and are thereby potential regulators of vascular and inflammatory disorders and disorders associated with, e.g., dysregulation of asparaginyl peptidase activity. For example, samples containing legumain and/or ZB-1 may be contacted with one of a plurality of test compounds (either biological agents or small organic molecules), and the activity of legumain and/or ZB-1 in each of the treated samples can be compared with the activity of legumain and/or ZB-1 in untreated samples or in samples contacted with different test compounds. Such comparisons will determine whether any of the test compounds results in: (1) a substantially decreased level of expression and/or activity (and/or secretion, if the sample consists of an intact cell(s)) of legumain and/or ZB-1, thereby indicating an antagonist of legumain and/or ZB-1; or (2) a substantially increased level of expression and/or activity (and/or secretion, if the sample consists of an intact cell(s)) of legumain or ZB-1, thereby indicating an agonist of legumain and/or ZB-1. In one embodiment, the identification of test compounds capable of modulating legumain and/or ZB-1 activity is performed using high-throughput screening assays, such as BIACORE® (Biacore International AB, Uppsala, Sweden), BRET (bioluminescence resonance energy transfer), and FRET (fluorescence resonance energy transfer) assays, as well as ELISA and cell-based assays.

As legumain hydrolyzes asparaginyl bonds (and ZB-1 is predicted similarly to hydrolyze such bonds), screens for antagonists of legumain and/or ZB-1 activity may employ well-established methods for analyzing the activity of a cysteine protease, or may follow the protocols described in the Examples. Thus, one may contact a cell or sample containing legumain and/or ZB-1 with a test compound, and determine if the test compound modulates legumain and/or ZB-1 expression by, e.g., Western or Northern Analysis, PCR, immunohistochemistry, in situ hybridization, differential display, etc. Alternatively, one may contact a cell or sample containing legumain and/or ZB-1 with a test compound and determine if the test compound modulates legumain and/or ZB-1 activity (and/or secretion, if the sample consists of an intact cell(s)). Legumain and/or ZB-1 activity may be measured by a variety of methods, including those disclosed in Chen et al. ((2006) supra), Li et al. (supra), and Kato et al. (supra). As shown in the examples, using, e.g., the peptide substrate Z-AAN-MCA (Peptide Institute), one may determine whether legumain or ZB-1 have increased or decreased protease activity in the presence of a particular test compound. Various direct and indirect legumain regulators are well known in the art, and may be used for comparative measurements (see, e.g., Vigreswaran et al., supra and Yamane et al., supra). In addition, activity-based probes for legumain are disclosed in Kato et al., supra.

Small Molecules

Decreasing legumain and/or ZB-1 activity, expression and/or secretion in an organism (or subject) afflicted with (or at risk for) a disorder related to enhanced legumain and/or ZB-1 expression and/or activity or a disorder related to, e.g., increased asparaginyl protease activity, e.g., atherosclerosis, arthritis, etc., or decreasing legumain and/or ZB-1 activity, expression, and/or secretion in a cell involved in such disorders from such an organism, may also be achieved through the use of small molecules (usually organic small molecules) that antagonize, i.e., decrease or inhibit the activity of, legumain and/or ZB-1. Novel antagonistic small molecules may be identified by the screening methods described herein and may be used in the methods of the present invention described herein. Additional small molecule regulators of legumain activity are well known in the art and may be used for comparative measurements or in the methods disclosed herein (see, e.g., Vigreswaran et al., supra; Niestro, et al., supra; and Götz, supra).

The term small molecule refers to compounds that are not macromolecules (see, e.g., Karp (2000) Bioinformatics Ontology 16:269-85; Verkman (2004) AJP-Cell Physiol. 286:465-74). Thus, small molecules are often considered those compounds that are, e.g., less than one thousand daltons (e.g., Voet and Voet, Biochemistry, 2^{nd} ed., ed. N. Rose, Wiley and Sons, New York, 14 (1995)). For example, Davis et al. ((2005) Proc. Natl. Acad. Sci. USA 102:5981-86) use the phrase small molecule to indicate folates, methotrexate, and neuropeptides, whereas Halpin and Harbury ((2004) PLos Biology 2:1022-30) use the phrase to indicate small molecule gene products, e.g., DNAs, RNAs and peptides. Examples of natural small molecules include, but are not limited to, cholesterols, neurotransmitters, aptamers, and siRNAs (see, Dykxhoom et al. (2006) Gene Therapy 13:541-52); synthesized small molecules include, but are not limited to, various chemicals listed in numerous commercially available small molecule databases, e.g., FCD (Fine Chemicals Database), SMID (Small Molecule Interaction Database), ChEBI (Chemical Entities of Biological Interest), and CSD (Cambridge Structural Database) (see, e.g., Alfarano et al. (2005) Nuc. Acids Res. Database Issue 33:D416-24).

Methods for Diagnosing, Prognosing, and Monitoring the Progress of Disorders and Conditions Related to Legumain and ZB-1 Activity

The present invention provides methods for diagnosing, prognosing, and monitoring the progress of disorders and conditions related to legumain and/or ZB-1 activity in a subject (e.g., conditions such as vascular and inflammatory disorders, which directly or indirectly involve increases in the activity of legumain and/or ZB-1) by detecting, e.g., an upregulation of legumain and/or ZB-1 activity, expression, and/or secretion, including, but not limited to, the use of such methods in human subjects. These methods may be performed by utilizing prepackaged diagnostic kits comprising at least one of the group comprising a legumain and/or a ZB-1 polynucleotide or fragments thereof, a legumain and/or a ZB-1 polypeptide or fragments thereof (including fusion proteins thereof), antibodies or antibody fragments to a legumain and/or a ZB-1 polypeptide, or small molecule modulators of legumain and/or a ZB-1 activity, expression, and/or secretion, as described herein, which may be conveniently used, for example, in a clinical setting. A skilled artisan will recognize that other indirect methods may be used to confirm, e.g., the upregulation of, e.g., human legumain and/or ZB-1, including, but not limited to, measuring changes in the mass or dimensions of an atherosclerotic plaque.

“Diagnostic” or “diagnosing” means identifying the presence or absence of a pathologic condition. Diagnostic methods include detecting a test amount of: 1) the level of expression of legumain and/or ZB-1; 2) the level of activity of legumain and/or ZB-1; and/or 3) the level of secretion of legumain and/or ZB-1, by determining a test amount of the level of expression of legumain and/or ZB-1 (e.g., the level of mRNA, cDNA, and/or polypeptide, including fragments thereof), level of activity of legumain and/or the ZB-1 (e.g., the level of asparaginyl protease/peptidase activity), and/or the level of secretion of legumain and/or ZB-1 (e.g., the level of legumain and/or ZB-1 found extracellularly) in a biological sample from a subject (human or nonhuman mammal), and comparing the test level/activity/secretion of legumain and/or ZB-1 with a normal level/activity/secretion of legumain and/or ZB-1 or range thereof (e.g., a reference amount, such as an amount or range from an individual(s) known not to suffer from disorders related to legumain and/or ZB-1 activity, etc.). Although a particular diagnostic method may not provide a definitive diagnosis of disorders related to legumain and/or ZB-1 activity, etc. it suffices if the method provides a positive indication that aids in diagnosis.

The present invention also provides methods for prognosing such disorders by detecting changes in legumain and/or ZB-1 expression, secretion, and/or activity. “Prognostic” or “prognosing” means predicting the probable development and/or severity of a pathologic condition. Prognostic methods include determining the test amount of: 1) the level of expression of a legumain and/or ZB-1 gene product; 2) the level of activity of legumain and/or ZB-1; and/or 3) the level of secretion of legumain and/or ZB-1 in a biological sample from a subject, and comparing the test level/activity/secretion of legumain and/or ZB-1 to a prognostic level/activity/secretion of legumain and/or ZB-1 or range thereof (e.g., an amount or range from individuals with varying severities of disorders related to legumain and/or ZB-1 activity, etc. and/or disorders associated with asparaginyl peptidase/protease dysregulation). In one embodiment, the prognostic level/activity/secretion of legumain and/or ZB-1 or range thereof may be a measurement from an individual at an earlier time point than the test level/activity/secretion of legumain and/or ZB-1. Various amounts of legumain and/or ZB-1 activity, secretion and/or expression in a test sample are consistent with certain prognoses for disorders related to legumain and/or ZB-1 activity and/or disorders associated with asparaginyl peptidase/protease dysregulation. The detection of an amount of legumain and/or ZB-1 activity, secretion and/or expression at a particular prognostic level provides a prognosis for the subject.

The present invention also provides methods for monitoring the progress or course of disorders, or the progress or course of treatment of disorders, related to legumain and/or ZB-1 activity (and/or disorders associated with asparaginyl peptidase/protease dysregulation, e.g., vascular disorders and inflammatory disorders) by detecting, e.g., the upregulation or downregulation of legumain and/or ZB-1 activity, secretion and/or expression. Monitoring methods include determining a test amount of: 1) the level of a gene product of legumain and/or ZB-1; 2) the level of activity of legumain and/or ZB-1; and/or 3) the level of secretion of legumain and/or ZB-1 in biological samples taken from a subject at a first and second time, and comparing the amounts. A change in the amount of legumain and/or ZB-1 activity, secretion, and/or expression between the first and second times indicates a change in the course of the legumain and/or ZB-1-related conditions or disorders. Such monitoring assays are also useful for evaluating the efficacy of a particular therapeutic intervention in patients being treated for legumain and/or ZB-1-associated conditions, and/or conditions resulting in asparaginyl peptidase/protease dysregulation.

Increased legumain and/or ZB-1 activity, secretion, and/or expression in the methods outlined above may be detected in a variety of biological samples, including bodily fluids (e.g., whole blood, plasma, and urine), cells (e.g., whole cells, cell fractions, and cell extracts), and other tissues. Biological samples also include sections of tissue, such as biopsies and frozen sections taken for histological purposes. Preferred biological samples include artery, kidney, blood vessels, endothelial cells, monocytes, and macrophages. It will be appreciated that analysis of a biological sample need not necessarily require removal of cells or tissue from the subject. For example, appropriately labeled agents that bind legumain and/or ZB-1 gene products (e.g., antibodies, nucleic acids) can be administered to a subject and visualized (when bound to the target) using standard imaging technology (e.g., CAT, NMR (MRI), and PET).

In the diagnostic and prognostic assays of the present invention, the level of legumain and/or ZB-1 activity, secretion, and/or expression is detected and quantified to yield a test amount. The test amount is then compared with a normal amount or range. Particular methods of detection and quantitation of the level of legumain and ZB-1 activity, secretion, and/or expression are described below.

Normal amounts or baseline levels of legumain or ZB-1 activity, secretion (i.e., location of gene products, e.g., secreted or extracellular versus intracellular), and/or expression may be determined for any particular sample type and population. Generally, baseline (normal) levels or the baseline locale of legumain and/or ZB-1 protein or mRNA are determined by measuring respective amounts or locale of legumain and/or ZB-1 protein or mRNA in a biological sample from normal (i.e., healthy) subjects. Alternatively, normal values of legumain and/or ZB-1 gene products or locale may be determined by measuring the level or locale in healthy cells or tissues taken from the same subject from which the diseased (or possibly diseased) test cells or tissues were taken. The amount of legumain and/or ZB-1 gene products (either the normal amount or the test amount) or the locale of such gene products (i.e., extracellular or intracellular) may be determined or expressed on a per cell, per total protein, or per volume basis. To establish a standard/control cell level or locale of legumain or ZB-1 in a sample, one can measure the level or locale of a constitutively expressed gene product or other gene product expressed at a known level or locale in cells of the type from which the biological sample was derived.

It will be appreciated that the assay methods of the present invention do not necessarily require measurement of absolute values of legumain and/or ZB-1 activity, secretion, and/or expression because relative values are sufficient for many applications of these methods. It will also be appreciated that in addition to the quantity or abundance of legumain and/or ZB-1 gene products, variant or abnormal legumain and/or ZB-1 gene products (e.g., mutated transcripts, truncated polypeptides) or their expression patterns may be identified by comparison to normal gene products and expression patterns.

Whether the expression or location of a particular gene product in two samples is significantly similar or significantly different, e.g., significantly above or significantly below a given level, depends on the gene itself and, inter alia, its variability in expression or localization between different individuals or different samples. It is within the skill of those in the art to determine whether expression levels or localization are significantly similar or different. Factors such as genetic variation, e.g., in legumain and/or ZB-1 expression levels or localization, between individuals, species, organs, tissues, or cells may be taken into consideration (if necessary) when determining whether the level of expression, activity, and/or secretion, e.g., of human legumain and/or ZB-1, between two samples is significantly similar or significantly different, e.g., significantly above or below a given level. As a result of the natural heterogeneity in gene expression between individuals, species, organs, tissues, or cells, phrases such as “significantly similar,” “significantly greater,” “significantly lower,” “significantly above” and the like cannot be defined as a precise percentage or value, but rather can be ascertained by one skilled in the art upon practicing the invention.

The diagnostic, prognostic, and monitoring assays of the present invention involve, e.g., detecting and quantifying, legumain and/or ZB-1 gene products in biological samples. Legumain and ZB-1 gene products include, but are not limited to, mRNAs and polypeptides; both can be measured using methods well known to those skilled in the art. For example, mRNA can be directly detected and quantified using hybridization-based assays, such as Northern hybridization, in situ hybridization, dot and slot blots, and oligonucleotide arrays. Hybridization-based assays refer to assays in which a probe nucleic acid is hybridized to a target nucleic acid. In some formats, the target, the probe, or both target and probe are immobilized. The immobilized nucleic acid may be DNA, RNA, or another oligonucleotide or polynucleotide, and may comprise naturally or normaturally occurring nucleotides, nucleotide analogs, or backbones. Methods of selecting nucleic acid probe sequences for use in the present invention are based on the nucleic acid sequences of legumain and ZB-1, and the methods are well known in the art.

Alternatively, mRNA can be amplified before detection and quantitation. Such amplification-based assays are well known in the art and include polymerase chain reaction (PCR), reverse-transcription-PCR (RT-PCR), quantitative or real time PCR (Q-PCR), PCR-enzyme-linked immunosorbent assay (PCR-ELISA), and ligase chain reaction (LCR). Primers and probes for producing and detecting amplified legumain and/or ZB-1 gene products (e.g., mRNA or cDNA) may be readily designed and produced without undue experimentation by those of skill in the art based on the nucleic acid sequences of legumain and ZB-1 provided herein and known in the art. As a nonlimiting example, amplified legumain or ZB-1 gene products may be directly analyzed, for example, by gel electrophoresis; by hybridization to a probe nucleic acid; by sequencing; by detection of a fluorescent, phosphorescent, or radioactive signal; or by any of a variety of well-known methods. In addition, methods are known to those of skill in the art for increasing the signal produced by amplification of target nucleic acid sequences. One of skill in the art will recognize that whichever amplification method is used, a variety of quantitative methods known in the art (e.g., quantitative PCR) may be used if quantitation of gene products is desired.

Legumain and/or ZB-1 polypeptides (or fragments thereof) may be detected using various well-known immunological assays employing the anti-legumain antibodies and anti-ZB-1 antibodies that may be generated as described herein. Anti-legumain antibodies have also been described in the literature (Choi et al. (1999) supra). Immunological assays refer to assays that utilize an antibody (e.g., polyclonal, monoclonal, chimeric, humanized, scFv, and/or fragments thereof) that specifically binds to, e.g., a human legumain or ZB-1 polypeptide (or a fragment thereof). Such well-known immunological assays suitable for the practice of the present invention include ELISA, radioimmunoassay (RIA), immunoprecipitation, immunofluorescence, fluorescence-activated cell sorting (FACS), and Western blotting. A legumain and/or ZB-1 polypeptide may also be detected using a labeled substrate for the protease, e.g., Z-AAN-MCA as shown in the examples, or the activity-based probes disclosed in Kato et al., supra. One of skill in the art will understand that the aforementioned methods may be applied to disorders and conditions related to legumain and/or ZB-1 activity, e.g., vascular and inflammatory disorders.

Uses of Molecules Related to Legumain and ZB-1 Activity in Therapy

The inventors have demonstrated the following: (1) legumain is highly expressed in human atherosclerotic samples relative to healthy arterial samples; (2) legumain expression in the aortic sinus and aortic arch of ApoE KO mice increases during atherosclerotic disease progression; (3) legumain is strongly positive in atherosclerotic lesions of the aortic arch and aortic sinus of ApoE−/− mice; (4) legumain expression in atherosclerotic lesions of the aortic sinus of ApoE−/− mice occurs in areas of inflammatory cell infiltration; (5) legumain expression in the coronary arteries of ApoE−/− mice increases in atherosclerotic plaques; (6) legumain expression is found within neointimal lesional areas of the carotid arteries in an ApoE−/− mouse model of accelerated atherosclerosis; (7) legumain is expressed in arterial endothelial cells of aortic sinus of ApoE−/− mice; (8) legumain is expressed in the kidney, e.g., in endothelial cells of renal arteries, and in proximal tubule cells; (9) legumain protein levels, mRNA levels, and activity is increased in differentiated THP-1 macrophages and M-CSF activated primary human macrophages; (10) legumain is found in the conditioned media of M-CSF-activated primary human macrophages; (11) legumain protein is present on the cell surface upon recombinant overexpression in CHO or HEK293 cells, and cell-surface legumain expressed by HEK293 cells is enzymatically active; (12) legumain is expressed in coronary arteries of an atherosclerotic patient; (13) legumain stimulation induces human monocyte migration; (14) legumain stimulation induces endothelial cell migration and proliferation in wound-healing models of HEK293 and HUVEC cultures, as well as endothelial cell invasion in HUVEC culture; (15) legumain expression is increased in the diseased paw of the collagen-induced arthritis (CIA) mouse model of arthritis; and (16) a novel splice variant of legumain, ZB-1, is secreted into the culture medium of recombinant ZB-1-overexpressing HEK293 cells.

The above results indicate that the disclosed methods for using molecules related to legumain and/or ZB-1 activity, e.g., antagonists of legumain and/or ZB-1, may be employed to treat legumain- and/or ZB-1-associated conditions and disorders, e.g., vascular and inflammatory disorders, such as atherosclerosis and arthritis. These methods will be particularly useful for treating such disorders in humans.

The legumain- and ZB-1-related molecules disclosed herein, including modulators of mouse and/or human legumain and/or ZB-1 polynucleotide and/or polypeptide activity, expression, and/or secretion, which may be identified using the methods described herein, may be used in vitro, ex vivo, or incorporated into pharmaceutical compositions and administered to individuals (e.g., human subjects) in vivo to treat, ameliorate, or prevent disorders related to legumain and/or ZB-1 activity and disorders related to asparaginyl peptidase/protease activity (e.g., vascular disorders and inflammatory disorders), by administration of a legumain and/or a ZB-1 antagonist(s) (e.g., legumain inhibitory polynucleotides (i.e., polynucleotides that decrease legumain levels, activity, and/or secretion either directly or indirectly), such as antisense molecules, siRNA molecules, and aptamers; legumain inhibitory polypeptides (i.e., polypeptides that decrease legumain and/or ZB-1 levels, activity, and/or secretion either directly or indirectly, e.g., fragments of legumain, such as fragments containing an inactive enzymatic domain, and fusion proteins thereof); antagonist anti-legumain and/or anti-ZB-1 antibodies or antibody fragments (i.e., antibodies or antibody fragments that decrease legumain and/or ZB-1 activity, expression, and/or secretion either directly or indirectly, including antibodies and antibody fragments that bind legumain and/or ZB-1 fragments); and antagonistic small molecules (e.g., siRNAs, aptamers, and small organic molecules or compounds)). Several pharmacogenomic approaches to consider in determining whether to administer a legumain and/or ZB-1 antagonist(s) are well known to one of skill in the art and include genome-wide association, candidate gene approach, and gene expression profiling. A pharmaceutical composition of the invention is formulated to be compatible with its intended route of administration (e.g., oral compositions generally include an inert diluent or an edible carrier). Other nonlimiting examples of routes of administration include parenteral (e.g., intravenous), intradermal, subcutaneous, oral (e.g., inhalation), transdermal (topical), transmucosal, and rectal administration. The pharmaceutical compositions compatible with each intended route are well known in the art.

A legumain and/or ZB-1 antagonist(s) may be used as a pharmaceutical composition when combined with a pharmaceutically acceptable carrier. Such a composition may contain, in addition to a legumain and/or a ZB-1 antagonist(s) (e.g., a human legumain antagonist), carriers, various diluents, fillers, salts, buffers, stabilizers, solubilizers, and other materials well known in the art. The term “pharmaceutically acceptable” means a nontoxic material that does not interfere with the effectiveness of the biological activity of the active ingredient(s). The characteristics of the carrier will depend on the route of administration.

The pharmaceutical composition of the invention may be in the form of a liposome in which a legumain and/or a ZB-1 antagonist(s) is combined, in addition to other pharmaceutically acceptable carriers, with amphipathic agents such as lipids that exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, etc.

As used herein, the term “therapeutically effective amount” means the total amount of each active component of the pharmaceutical composition or method that is sufficient to show a meaningful patient benefit, e.g., amelioration of symptoms of, healing of, or increase in rate of healing of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the methods of treatment or use of the present invention, a therapeutically effective amount of a legumain and/or ZB-1 antagonist(s) is administered to a subject, e.g., a mammal (e.g., a human). A legumain and/or ZB-1 antagonist(s) may be administered in accordance with the method of the invention either alone or in combination with other therapies, such as, e.g., in combination with additional therapies for arthritis or atherosclerosis. When coadministered with one or more agents, a legumain and/or a ZB-1 antagonist(s) may be administered either simultaneously with the second agent, or sequentially. If administered sequentially, the attending physician will decide on the appropriate sequence of administering the legumain and/or ZB-1 antagonist(s) in combination with other agents.

When a therapeutically effective amount of a legumain and/or ZB-1 antagonist(s) is administered orally, the binding agent will be in the form of a tablet, capsule, powder, solution or elixir. When administered in tablet form, the pharmaceutical composition of the invention may additionally contain a solid carrier such as a gelatin or an adjuvant. The tablet, capsule, and/or powder contain from about 5 to 95% binding agent, and preferably from about 25 to 90% binding agent. When administered in liquid form, a liquid carrier such as water, petroleum, oils of animal or plant origin such as peanut oil (exercising caution in relation to peanut allergies), mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol, or polyethylene glycol. When administered in liquid form, the pharmaceutical composition contains from about 0.5 to 90% by weight of the binding agent, and preferably from about 1 to 50% by weight of the binding agent.

When a therapeutically effective amount of a legumain and/or a ZB-1 antagonist(s) is administered by intravenous, cutaneous or subcutaneous injection, the legumain and/or ZB-1 antagonist(s) will be in the form of a pyrogen-free, parenterally acceptable aqueous solution. The preparation of such parenterally acceptable protein solutions, having due regard for pH, isotonicity, stability, and the like, is within the skill of those in the art. A preferred pharmaceutical composition for intravenous, cutaneous, or subcutaneous injection should contain, in addition to the legumain and/or ZB-1 antagonist(s), an isotonic vehicle such as sodium chloride injection, Ringer's injection, dextrose injection, dextrose and sodium chloride injection, lactated Ringer's injection, or other vehicle as known in the art. The pharmaceutical composition of the present invention may also contain stabilizers, preservatives, buffers, antioxidants, or other additive known to those of skill in the art.

The amount of a legumain and/or a ZB-1 antagonist(s) in the pharmaceutical composition of the present invention will depend upon the nature and severity of the condition being treated, and on the nature of prior treatments that the patient has undergone. Ultimately, the attending physician will decide the amount of legumain and/or ZB-1 antagonist(s) with which to treat each individual patient. Initially, the attending physician will administer low doses of legumain and/or ZB-1 antagonist(s) and observe the patient's response. Larger doses of legumain and/or ZB-1 antagonist(s) may be administered until the optimal therapeutic effect is obtained for the patient, and at that point the dosage is not generally increased further. It is contemplated that the various pharmaceutical compositions used to practice the method of the present invention should contain about 0.1 μg to about 100 mg of legumain and/or ZB-1 antagonist(s), e.g., human legumain polypeptides (including fusion proteins thereof), per kg body weight.

The duration of intravenous (i.v.) therapy using a pharmaceutical composition of the present invention will vary, depending on the severity of the disease being treated and the condition and potential idiosyncratic response(s) of each individual patient. It is contemplated that the duration of each application of the legumain and/or ZB-1 antagonist(s) may be within the range of, e.g., 1-12, 6-18, or 12-24 hrs of continuous or intermittent i.v. administration. Also contemplated is subcutaneous (s.c.) therapy using a pharmaceutical composition of the present invention. These therapies can be administered daily, weekly, or, more preferably, biweekly, or monthly. It is also contemplated that where the legumain and/or ZB-1 antagonist(s) is a small molecule (e.g., for oral delivery), the therapies may be administered daily, twice a day, three times a day, etc. Ultimately the attending physician will decide on the appropriate duration of i.v. or s.c. therapy, or therapy with a small molecule, and the timing of administration of the therapy, using the pharmaceutical composition of the present invention.

The polynucleotides and proteins of the present invention are expected to exhibit one or more of the uses or biological activities identified herein (including those associated with assays cited herein). Uses or activities described for proteins of the present invention may be provided by administration or use of such proteins or by administration or use of polynucleotides encoding such proteins (such as, for example, in gene therapies or vectors suitable for introduction of DNA).

Uses of Legumain and/or ZB-1 Antagonists

In one aspect, the invention features a method of regulating asparaginyl peptidase/protease activity in a cell or sample of interest (e.g., a monocyte, a foam cell, a macrophage, a kidney proximal tubule cell, a site of inflammatory cell invasion into a vessel, an atherosclerotic plaque intima, a kidney, or an artery). One such method comprises contacting a cell or population of cells with a legumain and/or a ZB-1 antagonist(s) (e.g., a human legumain and/or ZB-1 inhibitory polynucleotide or polypeptide (e.g., siRNA, aptamers, antisense, or antagonistic legumain and/or ZB-1 soluble proteins, including fusion proteins); or anti-legumain or -ZB-1 antibodies (i.e., antagonistic antibodies)) in an amount sufficient to modulate the level of asparaginyl peptidase/protease activity in the cell or sample of interest. In another embodiment of the invention, a legumain and/or ZB-1 antagonist(s) is used, such that the level of secretion or expression of legumain and/or ZB-1 is decreased in the cell or sample of interest. Modulation of asparaginyl peptidase/protease activity, expression and/or secretion is expected to be beneficial for individuals suffering from legumain-associated conditions, ZB-1-associated conditions, and/or conditions accompanied by asparaginyl peptidase/protease dysregulation, e.g., vascular and inflammatory disorders, such as atherosclerosis and arthritis.

Thus, antagonists of legumain and/or ZB-1 are believed to be useful to treat subjects afflicted with a condition such as atherosclerosis (including, but not limited to, all stages of atherogenesis and atherosclerosis, e.g., endothelial cell activation, formation of fatty streaks, inflammatory cell invasion of vessel walls, endothelial cell migration, formation of foam cells, plaque denudation, atheromatous plaque formation, atheromatous plaque rupture, atherothrombosis, aneurysm, stenosis, etc.), congestive heart failure, myocardial infarction, atrial and ventricular arrhythmias, stenosis, aneurysm, peripheral vascular disease, chronic peripheral arterial occlusive disease (CPAOD), peripheral artery occlusive disease (PAOD), thrombosis (including, e.g., acute arterial thrombosis, atherothrombosis, and deep venous thrombosis), embolism, inflammatory vascular disorders, Raynaud's phenomenon, vasculitis and/or arteritis (including, e.g., Bechet's disease, Buerger's disease, central nervous system vasculitis, Churg-Strauss syndrome cryoglobulinemia, giant cell arteritis, Kawasaki disease, microscopic polyangitis, polyarteritis nodosa, polymyalgia rheumatica, rheumatoid vasculitis, Takayasu's arteritis, and Wegener's granulomatosis), venous disorders, hypertensive vascular disease, claudication, stable angina, unstable angina, stroke, coronary artery disease (CAD), acute coronary syndrome (ACS), metabolic syndrome, ischemia, reperfusion, and exacerbation of various diseases affected by the circulatory system (e.g., chronic kidney disease, end-stage renal disease (ESRD), hyperlipidemia, hypertension, and diabetes). Additional disorders amenable to diagnosis, prognosis, monitoring, treatment, amelioration and/or prevention using the methods disclosed herein include inflammatory disorders (e.g., chronic inflammatory disorders, such as arthritis and tuberculosis).

The methods of the present invention are based, at least in part, on the finding that legumain expression is increased in atherosclerotic samples from the aortic arch, aortic sinus, carotid arteries foam cells, and sites of inflammatory cell infiltration into vessels, that legumain levels and activity are increased in activated macrophages, and that ZB-1 is a splice variant of legumain, which is secreted from ZB-1-overexpressing cells. Accordingly, legumain and/or ZB-1 antagonists, i.e., molecules that inhibit legumain and/or ZB-1 activity, expression and/or secretion (e.g., antagonist anti-legumain antibodies), may be used to decrease the asparaginyl peptidase/protease activity associated with vascular and inflammatory disorders, e.g., for treating, ameliorating, or preventing disorders such as atherosclerosis and arthritis.

By using a legumain and/or ZB-1 antagonist(s), it is possible to modulate asparaginyl protease/peptidase in a number of ways. For example, decreasing activity, expression and/or secretion may be undertaken by inhibiting or blocking an established legumain and/or ZB-1-associated condition or disorder, or may involve preventing the induction of a legumain and/or ZB-1-associated conditions or disorders.

Pharmaceutical compositions of the invention containing a legumain and/or ZB-1 antagonist(s) may also contain additional therapeutic agents for treatment of the particular targeted disorder. For example, a pharmaceutical composition for treatment of atherosclerosis may also include anti-hypertensive agents, cholesterol-reducing drugs, statins, and/or inflammatory cytokine mediators, such as HUMIRA® or ENBREL®. The pharmaceutical composition may contain thrombolytic or antithrombotic factors such as plasminogen activator and Factor VIII. The pharmaceutical composition may further contain additional anti-inflammatory agents. Such additional factors and/or agents may be included in the pharmaceutical composition to produce a synergistic effect with legumain and/or ZB-1 antagonist(s), or to minimize side effects caused by the legumain and/or ZB-1 antagonist(s).

In one embodiment, a legumain and/or ZB-1 antagonist(s), including pharmaceutical compositions thereof, is administered in combination therapy, i.e., combined with other agents, e.g., therapeutic agents, that are useful for treating pathological conditions or disorders, such as disorders of the cardiovascular system. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

Preferred therapeutic agents used in combination with a legumain and/or ZB-1 antagonist(s) are those agents that modulate different aspects of vascular and inflammatory disorders, e.g., agents that interfere with the activity of proinflammatory cytokines.

Thus, agents useful in combination with a legumain and/or an ZB-1 antagonist(s) include, without limitation, agents that stimulate cholesterol efflux from cholesterol containing cells, e.g., macrophages and foam cells, such as modulators of PPARs (i.e., PPARα, PPARβ, and PPARγ), modulators of LXR (Liver X Receptor, e.g., oxysterols), and modulators of ABC (ATP-binding cassette transporters, e.g., ABCA, ABCG, and ABC8). Such agents include thiazolidinediones (e.g., glitazones, such as rosiglitazone and troglitazone), fatty acids (including polyunsaturated fatty acids), fibrates (e.g., fenofibrate, gemfibrozil, clofibrate, Wy-14,643), GW1516, GW764, GW7845, GWO742, GW7647, eicosapentaenoic acid, xanthohumols, roselipins, prenylflavonoids, polyacetylenes, tanshinones and derivatives thereof (see, e.g., Coleman and Lee (2004) Prog. Lipid Res. 43:134-76; Chen and Farse (2005) Atheroscler. Thromb. Vasc. Biol. 25:482-86; Rustan et al. (1988) J. Lipid Res. 29:1417-26; Tabata et al. (1997) Phytochemistry 46:683-87; Tomoda (1999) J. Antibiotics 52:689-94; Chung et al. (2004) Planta Med. 70:258-60; Lee et al. (2004) Planta Med. 70:197-200; Ko et al. (2002) Arch. Pharm. Res. 25:446-48; Li et al. (2004) J. Clin. Invest. 1564-76; Castrillo and Tontonz (2004) J. Clin. Invest. 114:1538-40; Marx et al. (2004) Circ. Res. 94:1168-78; Chawla et al. (2001) Mol. Cell. 7:161-71; Lie et al. (2000) J. Clin. Invest. 106:523-31; Collins et al. (2001) Artheroscler. Thromb. Vasc. Biol. 21:365-71; Lee et al. (2003) Science 302:453-57; Duez et al. (2002) J. Biol. Chem. 277:48051-57; Rubins et al. (1999) N. Eng. J. Med. 341:410-18; Oliver et al. (2001) Proc. Natl. Acad. Sci. USA 98:5306-11; Chinetti et al. (2001) Nat. Med. 7:53-58; Ricotte et al. (1998) Nature 391:79-82; Joseph et al. (2002) Proc. Natl. Acad. Sci. USA 99:7604-09; Tangirala et al. (2002) Proc. Natl. Acad. Sci. USA 99:11896-901; Venkateswaran et al. (2000) J. Biol. Chem. 275:14700-07; and Wang et al. (2004) Proc. Natl. Acad. Sci. USA 101:9774-79).

Additionally, combination therapy with legumain and/or ZB-1 antagonist(s) may utilize statins (e.g., mevastatin, lovastatin, simvastatin, pravastatin, fluvastatin, atorvastatin, cerivastatin and rosuvastatin), cystatins (e.g., ovocystatin, cystatin C, and cystatin M), and/or agents that augment statin activity and/or bioavailability, such as inhibitors of cytochromes P 450 (CYP 450), CYP 3A4, and CYP 2C8/9 (e.g., macrolide antibiotics, azoles, protease inhibitors, verapamil, diltiazem, amiodarone, warfarin, grapefruit juice, gemfibrozil, phenyloin, losartan, diclofenac, ibuprofen, and dolbutamide), and inhibitors of the hepatic statin transporter, OATP-C (e.g., cyclosporine A and gemfibrozil) (see Rutishauser (2006) Swiss Med. Wkly. 136:41-49). Additional agents useful in combination with legumain and/or ZB-1 antagonist(s) include agents that reduce hyperglycemia (e.g., repaglinide, see, Schmoelzer and Wascher (2006) Cardiovasc. Diabetol. 5:9); antagonists of leukotrienes (e.g., antagonists of proteins involved in leukotriene biosynthesis, such as 5-lipoxyegenase (5-LO), 5-LO-activating protein (FLAP), and leukotriene hydrolases (e.g., LTA4 hydrolase)); agents that lower cholesterol, LDL, and triglyceride levels (e.g., fibrinates, HMG-CoA reductase inhibitors, nicotinic acid derivatives); anti-hypertensive agents; anti-platelet agents; anti-coagulants; inhibitors of cholesterol acyltransferase enzymes (see, Krause et al. (1995) Inflammation Mediators and Pathways, pp. 173-98 CRC Press, Boca Raton, Fla.); agents for the treatment of diabetes (e.g., insulin; insulin sensitizers such as metformin; Glp-1 mimetics, such as exenatide (BYETTA®); insulin secretagogues, such as sulfonylureas (e.g., tolazamide, glyburide and others) and metiglinides (e.g., nateglinide (STARLIX®)); modulators of sterol regulatory element-binding protein (SREBP), such as atorvastatin and simvastatin (e.g., LIPITOR® and CADUET®); modulators of farnesoid X receptor (FXR) (e.g., bile acids); and other modulators of tissue lipid and cholesterol levels.

Another aspect of the present invention accordingly relates to kits for carrying out the administration of a legumain and/or ZB-1 antagonist(s) with other therapeutic compounds. In one embodiment, the kit comprises one or more legumain and/or ZB-1 antagonists formulated with one or more binding agents in a pharmaceutical carrier, and at least one other agent, e.g., another therapeutic agent, formulated as appropriate, in one or more separate pharmaceutical preparations.

Involvement of Legumain in Vascular and Inflammatory Disorders

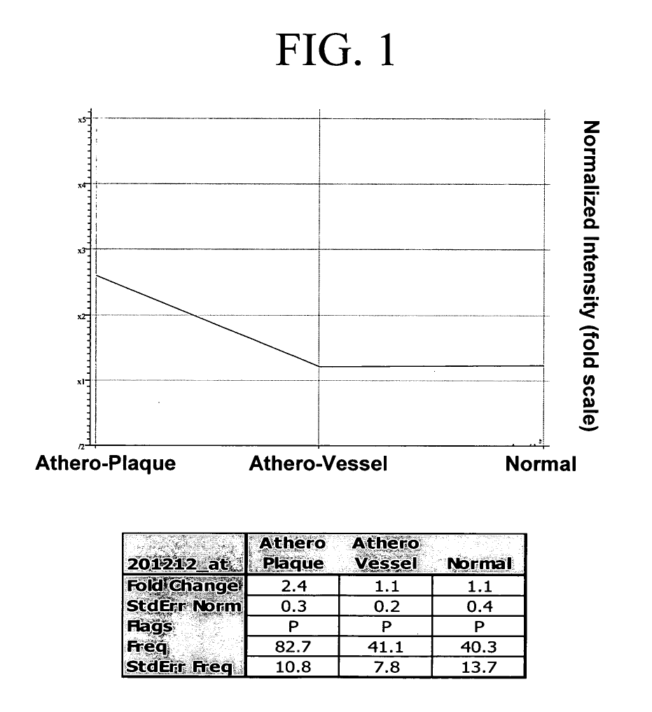

The present invention provides methods of treating, ameliorating, or preventing vascular disorders, e.g., atherosclerosis, comprising contacting a cell or cell population with a modulator of the lysosomal cysteine protease legumain and/or ZB-1, and/or comprising administering such a modulator to a subject. Lysosomal cysteine protease-mediated proteolysis is associated with multiple pathological aspects of atherogenesis. The inventors have shown that the cysteine protease legumain is highly expressed in the atherosclerotic plaques in ApoE−/− mice, as well as in lesions formed in ligated mouse carotid arteries. In the atherosclerosis-prone ApoE−/− mice, progressive increases in legumain expression in lesioned areas correlated with disease advancement. Legumain protein was not observed in normal vascular tissues, and was first detected in the developing lesion in 2-month old ApoE−/− mice. Prominent legumain expression was found in the advanced atherosclerotic lesions in 6-month and 1-year old ApoE−/− mice. Consistent with the results from ApoE−/− mice, data mining using the GENELOGIC® database revealed a 2.4-fold increase in legumain mRNA expression in human atherosclerotic plaques (FIG. 1) compared with either asymptomatic blood vessels from the same patient, or blood vessels from asymptomatic patients. Thus, the pattern of legumain expression suggested its involvement in vascular inflammatory disorders, e.g., atherosclerosis.

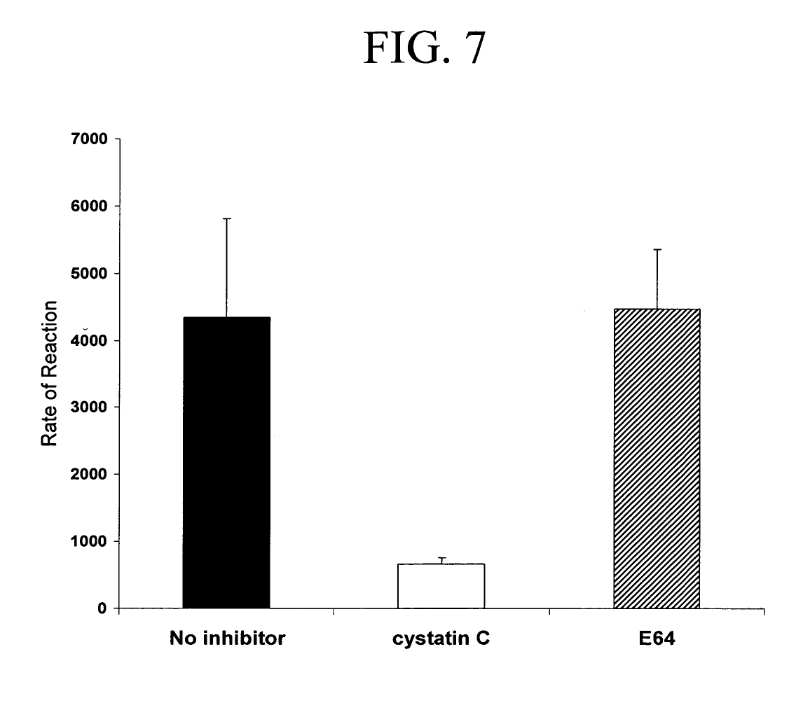

Also disclosed herein is the finding that macrophages are the major cell type expressing disease-associated legumain in atherosclerotic plaques. Macrophages are a rich source of extracellular lysosomal proteases with implications in extracellular matrix remodeling and plaque destabilization. Reddy et al. have shown that monocyte-derived macrophages secrete active cathepsin S and L into the extracellular milieu that can participate in vascular remodeling (Reddy et al. (1995) Proc. Natl. Acad. Sci. U.S.A. 92:3849-53). Others have confirmed that cathepsins S and L can functionally promote atherosclerosis (Liu et al. (2006) Atherosclerosis 184:302-11; Sukhova et al., supra). Disclosed herein are the findings that differentiated macrophages express high levels of legumain and are capable of secreting legumain into the extracellular space. Although the secreted legumain was found as the pro-form, it may become active in the extracellular acidic microenvironment where it activates other macrophage-derived lysosomal proteases, including cathepsins B, L, and S (e.g., Reddy et al., supra). Alternatively, legumain may be activated in intracellular compartments and then become associated with the cell surface. Indeed, disclosed herein is the finding that 293 cells overexpressing legumain exhibited cell-surface legumain activity (e.g., FIG. 7; see also Liu et al. (2003) Cancer Res. 63:2957-64). This activity may result from active legumain in endosomes/lysosomes being presented on the cell surface as a result of endosomal/lysosomal membranes fusing with the plasma membrane (Andrews (2000) Trends Cell Biol. 10:316-21). The cell-membrane association may be mediated by legumain binding to integrins via the RGD sequence in the mature legumain enzyme.

Endogenous cell-surface legumain activity can be difficult to detect, and may only be present in the lesion microenvironment. For example, tumor cell surface-associated legumain has only been found in the tumor microenvironment, but not on tumor cells in culture (Liu et al., supra; Wu et al. (2006) Cancer Res. 66:970-80). Furthermore, legumain was detected on the surface of tumor-associated macrophages, but not on circulating monocytes (Wu et al., supra).

A proposed role for other extracellular lysosomal cysteine proteases in atherosclerosis is extracellular matrix (ECM) degradation and tissue remodeling. Cathepsins L and S possess collagenolytic and elastolytic activities that are directly involved in ECM degradation (Liu et al. (2006) supra; Reddy et al., supra). In addition, several cathepsins, including cathepsins B and L, may process caspases and induce apoptosis, leading to atherosclerotic lesion evolution (Guicciardi et al. (2000) J. Clin. Invest. 106:1127-37; Ishisaka et al. (1999) Cell Struct. Funct. 24:465-70; Vancompemolle et al. (1998) FEBS Lett. 438:150-58). ECM components and caspases are not currently known to be legumain substrates. However, legumain may indirectly contribute to ECM degradation by processing and activating other collagenolytic/elastolytic enzymes, such as MMP-2 and cathepsins B, H, and L. The presence of legumain at atherosclerotic plaques together with its proteolytic function suggest that modulators of legumain expression and activity are useful in methods of treating, preventing, or ameliorating atherosclerosis.

Macrophages are postulated to damage host tissues in chronic inflammatory disease states by causing degradation of the surrounding tissue (Reddy et al., supra). The results provided herein indicate that legumain is expressed in the arthritic paw of the mouse CIA model. As rheumatoid arthritis is a chronic inflammatory disease, the discovery that legumain is expressed in arthritic joints in the CIA model suggests a role for legumain-mediated joint degradation, possibly via macrophage activity. In addition, it is important to note that several vascular disorders are also inflammatory disorders, i.e., the inflammation of, e.g., the intima of the vessel is at least in part responsible for the damage to the vasculature. Thus, modulators of legumain can be used in methods of treating, preventing, or ameliorating inflammatory disorders, e.g., rheumatoid arthritis.

Cell migration is a major feature of inflammatory diseases, including but not limited to vascular inflammatory disorders, e.g., atherosclerosis. For instance, migration of monocytes into the arterial wall is an early event of the atherosclerotic process leading to the formation of foam cells and ultimately to the development of advanced atherosclerotic lesions. As disclosed herein, legumain can induce migration of monocytes in nanomolar concentrations, and the chemoattractant activity of legumain can be as potent as VEGF. This suggests legumain possesses chemoattractant properties.

Monocyte invasion of the endothelial cell layer is thought to occur by the process known as extravasation (e.g., leukocyte extravasation), in which monocytes first adhere to the endothelial cell layer of the blood vessel, and then squeeze between endothelial cells towards the basement membrane and the vessel neointima (Janeway et al. (1999) Immunobiology, 4^{th} Edition, p. 607, Elsevier Science Ltd./Garland Publishing). Because the present invention discloses that legumain induces monocyte recruitment to sites of atherosclerotic lesions, antagonists of legumain and/or ZB-1 will be useful in preventing both monocyte recruitment and monocyte extravasation. Monocytes that have successfully extravasated into the neointima usually differentiate into macrophages; thus, legumain and/or ZB-1 antagonists will also prevent monocyte differentiation at the sites of atherosclerotic plaques.

Furthermore, these same data suggest that legumain and/or ZB-1 agonists and antagonists will be useful in promoting and inhibiting, respectively, other forms of extravasation, e.g., cancer metastasis (see further below), other forms of leukocyte extravasation, etc. One of ordinary skill in the art will know of several established assays for evaluating the biological effects of, e.g., legumain agonists or antagonists on cellular processes/activities such as cell migration, extravasation, etc., in addition to such related assays presented in the Examples (below).

The chemoattractant function of legumain appears to be independent from its protease activity because both the purified form of legumain and heat-denatured form retain the chemoattractant function (data not shown), which suggests that a linear peptide sequence mediates the chemotactic function of legumain. Interestingly, a protease-independent biological activity has been described for legumain. The inactive proform of legumain contains a 17-kDa C-terminal peptide, OIP-2 (osteoclast inhibitory peptide 2) that is cleaved during autocatalytic activation of legumain. Legumain/OIP-2 has been shown to inhibit the differentiation of monocytes into osteoclasts, as well as inhibit bone resorption (Choi et al. (2001) supra; Choi et al. (1999) supra). Thus, legumain receptor may be present on the surface of monocytes, and the C-terminal peptide of legumain may be sufficient to mediate chemoattraction. As such, legumain may exhibit dual functions in atherogenesis, e.g., as a protease and as a chemoattractant. Legumain protease function may lead to extracellular matrix degradation, whereas the chemoattractant function may contribute to monocyte recruitment into atherosclerotic lesions as well as macrophage retention in the plaque. Because of the dual function of legumain, antagonists of legumain will inhibit both proteolytic and chemotactic functions of legumain, and thus will be useful in the methods of treatment of, e.g., atherosclerosis. In addition, OIP-2 and related agonists and/or antagonists may be useful in treating, ameliorating, or preventing various vascular disorders or inflammatory disorders in, e.g., a mammal.

In addition, the invention teaches that legumain is expressed by endothelial cells of ApoE−/− mice, and this result is consistent with detection of legumain in the surface of tumor vascular endothelial cells (Wu et al., supra). As disclosed herein, endothelial cells, e.g., HEK293 cells and HUVECs, have increased migratory properties in response to legumain. This result, combined with the detection of a high concentration of inflammatory cells expressing legumain in regions of plaque neovascularization in human coronary arteries, suggests a role of legumain in angiogenesis. Intra-plaque angiogenesis is believed to be a critical pathological feature of atherosclerosis, enhancing plaque growth and vulnerability (Moulton et al (2003) Proc. Natl. Acad. Sci. 100:4736-41; Moulton et al. (1999) Circulation 99:1726-32). Legumain secreted by macrophages may contribute to neovessel formation by promoting endothelial cell migration, invasion and proliferation; therefore modulators of legumain may be useful in the methods of treating, ameliorating, or preventing angiogenesis, e.g., angiogenesis associated with atherosclerosis and tumor growth, as well as methods of inhibiting or promoting proliferation of endothelial cells (e.g., promoting proliferation, and angiogenesis, in revascularization of tissue).

Interestingly, legumain was recently found to be present and active in the microenvironment of tumor cells, in association with the extracellular matrix and cell surface. It has been suggested that extracellular legumain activity may functionally contribute to the metastatic behavior of tumor cells. Indeed, researchers have shown that legumain is found in membrane-associated vesicles at the invadopodia of tumor cells, as well as on the surface of tumor cells where it colocalizes with integrins (Liu et al. (2005) Cancer Res. 63:2957-64; Wu et al. (2006) Cancer Res. 66:970-80). Legumain has also been reported to play a role in tumor pathology (Murthy et al. (2005) Clin. Cancer Res. 11:2293-99). As disclosed herein, legumain plays a key role in angiogenesis, by promoting endothelial cell migration. Thus, legumain activity will be important in promoting angiogenesis related to metastatic cancers; and antagonists of legumain and/or ZB-1 may be useful in the methods of treating, ameliorating, and preventing tumor metastasis.

Conversely, agonists of legumain and/or ZB-1 may be useful in methods of treating, ameliorating, or preventing conditions requiring increased vessel formation. For example, agonists of legumain may be used in methods of promoting, e.g., revascularization, wound healing, transplant surgery recovery, etc.

In summary, the data provided herein establishes a novel link between the lysosomal protease legumain and vascular and inflammatory disorders. These results also show that monocytes/macrophages are a major source of atherosclerosis-associated legumain, and that cell surface/extracellular legumain may functionally contribute to disease formation, e.g., through stimulation of cell migration.

The entire contents of all publications, patents, patent applications, and other references cited throughout this application are hereby incorporated by reference herein in their entireties.

### EXAMPLES

The following Examples provide illustrative embodiments of the invention and do not in any way limit the invention. One of ordinary skill in the art will recognize that numerous other embodiments are encompassed within the scope of the invention.

The Examples do not include detailed descriptions of conventional methods, such as methods employed in the construction of vectors, the insertion of genes encoding polypeptides into such vectors and plasmids, the introduction of such vectors and plasmids into host cells, and the expression of polypeptides from such vectors and plasmids in host cells. Such methods are well known to those of ordinary skill in the art.

### Example 1

### Legumain is Highly Expressed in Human Atherosclerotic Samples and During Atherosclerosis Disease Progression in ApoE−/− Mice

To determine whether cysteine proteases might be involved in atherosclerotic lesions, the expression pattern of legumain was analyzed in human atherosclerotic arterial samples.

### Example 1.1

### Expression Profiling of Legumain in Human Atherosclerosis

Expression data of human atherosclerotic plaques and human plaque-free arterial samples was downloaded from the GENELOGIC™ database. The data were generated from hybridization of RNA to the AFFYMETRIX® (Santa Clara, Calif.) Hg_{—}133A GENECHIP™ oligonucleotide microarrays. Data analysis was performed using GENESPRING™. The normalized data were filtered for gene transcripts that had either increased or decreased levels of expression relative to the average of the controls. Gene transcripts with increased levels of expression had to have a call of “Present,” a frequency >5, and a change in expression of at least 2-fold in at least 70% of the samples. Decreasing gene transcripts had to have a call of “Present,” a frequency >5, and a change in expression of at least 2-fold in at least 70% of the samples. The statistical analyses were performed using GENESPRING™ v6.1.

### Example 1.2

### Results

Legumain expression was increased in human atherosclerotic arterial samples containing plaques relative to plaque-free segments or nondiseased arterial samples (FIG. 1).

### Example 2

### Legumain is Highly Expressed in Atherosclerotic Lesions of the Aortic Arch, Coronary Arteries, and the Aortic Sinus in ApoE−/− Mice

Gene expression associated with disease progress in atherosclerosis-prone Apolipoprotein E-deficient (ApoE−/−) mice was determined by microarray analysis. The ApoE−/− mice develop severe hypercholesterolemia that induces the formation of atherosclerotic lesions at specific locations in the vasculature, including the aortic sinus, the aortic arch and the proximal portion of the coronary arteries (Nakashima et al. (1994) Arterioschler. Thromb. 14:133-40; Reddick et al. (1994) Arterioscler. Thromb. 14:141-47).

### Example 2.1

### Animals and Tissue Preparation

All animal studies were approved by the Institutional Animal Care and Use Committee. Male ApoE KO (ApoE−/−) and C57BL/6 mice from Taconic Farms (Germantown, N.Y.) were maintained on a normal chow diet and euthanized at selected time points. All mice were sacrificed by inhalation of 100% CO₂ and perfused with saline solution injected through the left ventricle. Heart and aortic arch were further perfused with RNALATER® (Ambion Inc., Austin, Tex.) collected and frozen in preparation for gene expression studies. For histological studies, perfusion with saline through the left ventricle was followed by perfusion with 4% paraformaldehyde. Heart, aortic arch, and kidneys (see below) were stored overnight in 4% paraformaldehyde, switched to 70% ethanol, dehydrated and embedded in paraffin blocks in preparation for in situ hybridization or immunohistochemistry. Paraffin embedded heart samples were sectioned as previously described in order to collect tissue sections located within the aortic sinus (Paigen et al. (1987) Atherosclerosis 68:231-40).

### Example 2.2

### Affymetrix Hybridization and Analysis

### Example 2.2.1

### RNA Analysis

Total RNA was isolated and purified from pooled aortic arches (n=3-5) collected at selected time points in ApoE KO (ApoE−/−) and C57BL/6 mice. Total RNA was isolated using RNAEASY™ minikit sample lysis buffer (RLT), and RNA was purified according to the manufacturer's recommendations (Qiagen, Valencia, Calif.). For each sample, total RNA was quantitated from a measure of UV absorption at 260 nm, and an aliquot was analyzed with an Agilent® 2100 BIOANALYZER™ (Agilent Technologies, Palo Alto, Calif.) to determine RNA integrity.

### Example 2.2.2

### Preparation of Hybridization Solutions for Oligonucleotide Array Analysis

Double-stranded cDNA was prepared from 3-5 μg of total RNA using the SUPERSCRIPT™ Choice kit (Invitrogen, Carlsbad, Calif.) and 33 pmol of oligo-dT primer containing a T7 RNA polymerase promoter (Proligo, LLC, Boulder, Colo.). First strand cDNA synthesis was initiated with the addition of the following kit components: first strand buffer at 1×, DTT at 10 mM, dNTPs at 500 mM, SUPERSCRIPT™ RT II at 400 U, and RNAse inhibitor at 40 U. The reaction proceeded at 47° C. for 1 hour. Second strand synthesis proceeded with the addition of the following kit components: second strand buffer at 1×, additional dNTPs at 200 mM, E. coli DNA polymerase I at 40 U, E. coli RnaseH at 2 U, E. coli DNA ligase at 10 U. The reaction proceeded at 15.8° C. for 2 hr. T4 DNA polymerase (New England Biolabs, Beverly, Mass.), at a final concentration of 6 U (2 μl of 3000 Upper ml stock), was added for the last five minutes of the second strand reaction. Double stranded cDNA was purified using the GENECHIP® Sample Cleanup Module as described by the manufacturer (Affymetrix, Santa Clara, Calif.). Purified cDNA (10 μl) was transcribed with the Bioarray High Yield RNA TRANSCRIPT LABELING KIT (T7)™, following the manufacturer's protocol (Enzo, Farmingdale, N.Y.). Biotin-labeled, antisense cRNA was purified using the GENECHIP® Sample Cleanup Module as described by the manufacturer (Affymetrix, Santa Clara, Calif.). The cRNA yield was determined from a measure of UV absorption at 260 nm.

### Example 2.2.3

### Oligonucleotide Microarray Hybridization Procedures

Fragmented cRNA (15 μg) was prepared as previously described (Byrne et al. (2000) Preparation of mRNA for expression monitoring, In: Ausubel et al. (eds.). Current Protocols in Molecular Biology, John Wiley and Sons, Inc., New York) and used to create an oligonucleotide microarray hybridization solution as suggested by the manufacturer (Affymetrix, Santa Clara, Calif.). Hybridization solutions contained a mix of eleven prokaryotic RNAs (Hill et al., supra), each at a different known concentration, which were used to create an internal standard curve for each microarray and interpolated to determine the frequencies of detected genes. The hybridization solution was heated for 1-2 min at 95° C. and microcentrifuged at maximum speed for 2 minutes to pellet insoluble debris. Labeled cRNA solutions were hybridized to AFFYMETRIX® (Santa Clara, Calif.) Mouse Genome 430 2.0 GENECHIP™ oligonucleotide microarrays. Following hybridization, cRNA solutions were recovered and microarrays were washed and prepared for scanning according to Affymetrix protocols. Raw fluorescence data were collected and reduced with the use of the GENECHIP™ MAS 5.0 software application (Affymetrix, Santa Clara, Calif.). Frequency was determined using a bacterial RNA standard curve (Hill et al. (2001) Genome Biol. 2(12):researchO055.1-0055.13).

### Example 2.2.4

### Analysis of Expression Profiling Data

Data were reduced by filtering for gene transcripts that had either increased or decreased levels of expression relative to the average of the controls. Gene transcripts with increased levels of expression had to have a call of “Present,” a frequency >5, and a change in expression of at least 2-fold in at least 70% of the samples. Decreasing gene transcripts had to have a call of “Present” and a frequency >5 in at least 70% of the controls, and a change in expression of at least 2-fold in at least 70% of the samples. The statistical analyses were analyzed by GENESPRING™ v6.1 (Agilent Technologies, Palo Alto, Calif.) using a Welch ANOVA and several multiple testing corrections (Benjamini and Hochberg False Discovery Rate and Bonferroni Multiple Testing Correction—Family-wise error rate (FWER) p<0.05). The legumain qualifier was chosen for additional analysis.

### Example 2.3

### TAQMAN® Real-time Quantitative PCR

RNA was isolated and purified from mouse tissues (or THP-1 cells; see below) using RNEASY® kit (Qiagen, Valencia, Calif.) according to the manufacturer's instructions. Using an ABI PRISM® 7000 Sequence Detection System (PE Applied Biosystems, Foster City, Calif.), legumain mRNA levels were measured by TAQMAN® real-time quantitative PCR as previously described (Lake et al. (2005) J. Lipid Res. 46:2477-87) with Assay-on-Demand TAQMAN® reagents (PE Applied Biosystems, Foster City, Calif. or Eurogentec, San Diego, Calif.). The following primers were used: CCAGGÀGGCTGTAACCCACTT (forward primer; SEQ ID NO:14) and GCAAGGCATGCTCGTACGT (reverse primer; SEQ ID NO:15). Data were analyzed according to the manufacturer's instructions.

### Example 2.4

### In Situ Hybridization

Murine legumain sense and antisense riboprobes were produced by generating two independent PCR products with T7 RNA polymerase binding sites at either the 5′ end of the PCR product for sense riboprobe or the 3′ end of the PCR product for antisense riboprobe. Digoxigenin-labeled probes were prepared as described by the manufacturer using DIG RNA labeling mix and T7 RNA polymerase (Roche Diagnostics, Mannheim, Germany). Primer and probe sequences are described in Table 2 (below).

Sections of paraffin-embedded tissue were deparaffinized with xylene (2 changes, 3 minutes each) and rehydrated in water. After a rinse in RNase-free water and phosphate buffered saline (PBS), permeabilization was performed by incubation with 0.2% Triton-X 100/PBS for 15 minutes. After 2 washes with PBS, each at 3 minutes, the sections were ready for proteinase K (PK) (Sigma, St. Louis, Mo.) treatment. Sections were immersed in 0.1M Tris and 50 mM EDTA (Sigma) (pH 8.0) prewarmed at 37° C. containing 5 mg/ml PK for 15 minutes. PK activities were stopped by 0.1M glycine/PBS for 5 minutes followed by a post-fixation with 4% paraformaldehyde for 3 minutes and a PBS rinse. To prevent nonspecific electrostatic binding of the probe, sections were immersed in 0.25% acetic anhydride and 0.1M triethanolamine solution (pH 8.0) for 10 minutes, followed by 15 seconds in 20% acetic acid at 4° C. After 3 changes in PBS, 5 minutes each, sections were dehydrated through 70%, 90% and 100% ethanol, each at 3 minutes. The sections were completely air dried before 40 ml of prehybridization buffer was applied, covered with Parafilm and incubated at 52° C. for 30 minutes to reduce nonspecific binding. Parafilm was removed and 40 ml of hybridization buffer containing 5 ng/ml of digoxigenin-labeled probe was applied to each section, recovered with Parafilm and incubated overnight at 52° C. in a humid chamber.

The Parafilm was carefully removed and the sections were placed in a GENOMX™ i6000 (Biogenex, San Ramon, Calif.) automatic staining system. Sections were washed in 2× saline sodium citrate (SSC)/0.1% lauryl sulphate (SDS) (both Sigma) at room temperature, 4 changes, at 5 min each. To ensure only specific hybridization signal remains, sections were washed in a high stringency solution containing 0.1×SSC/0.1% SDS at 52° C., 2 changes, 5 minutes each. To reduce endogenous peroxidase staining, the sections were incubated in 3% H₂O₂ for 15 minutes followed by 3 washes in buffer. The labeled probe was detected with anti-digoxigenin antibody conjugated to horseradish peroxidase complex (Roche, Nutley, N.J.) diluted 1:500 in 2% normal sheep serum/0.1% Triton X-100 for 1 hours. The biotinylated complex was amplified using a Tyramide Amplification System (TSA™) (Biogenex, San Ramon, Calif.). Sections were incubated with TSA for 5 minutes, followed by 5 washes in buffer. The TSA complex was then amplified further by incubation with horseradish peroxidase for 15 minutes, followed by 5 washes in buffer. The amplified product was developed with 3,3′-diaminobenzidine (Vector Laboratory, Burlingame, Calif.) for 10 minutes, washed in water, stained briefly with Mayers' hematoxylin (Sigma), dehydrated through graded alcohol into xylene, and mounted in a DPX mountant before microscopic examination.

TABLE 2
Sense riboprobe

Forward Primer

(SEQ ID NO:16)

5′GACTGATAATACGACTCACTATAGGGCGAACACCAACACCAGCCATGT
C3′
Reverse Primer

(SEQ ID NO:17)

5′CTCTCAGCAGTTTCCCCAAATC3′
Sequence, 313NTs

(SEQ ID NO:18)

acaccaacac cagccatgtc atgcaatatg ggaacaaatc
tatctctacc atgaaagtga tgcagtttca gggaatgaag
cacagagcca gttcccccat ctccctgcct ccggtcacac
accttgacct cacccccagc cctgacgtgc ccctgaccat
cttgaagagg aagctgctga gaaccaacga cgtgaaggaa
tcccagaatc tcattgggca gatccagcaa tttctggatg
ccaggcacgt cattgagaag tctgtgcaca agatcgtttc
cctgctggcg ggatttgggg aaactgctga gag
Antisense riboprobe
Forward Primer

(SEQ ID NO:19)

5′ACACCAACACCAGCCATGTC3′
Reverse Primer

(SEQ ID NO:20)

5′GACTGATAATACGACTCACTATAGGGCGACTCTCAGCAGTTTCCCCAA
ATC3′
Sequence, 313NTs

(SEQ ID NO:21)

ctctcagcag tttccccaaa tcccgccagc agggaaacga
tcttgtgcac agacttctca atgacgtgcc tggcatccag
aaattgctgg atctgcccaa tgagattctg ggattccttc
acgtcgttgg ttctcagcag cttcctcttc aagatggtca
ggggcacgtc agggctgggg gtgaggtcaa ggtgtgtgac
cggaggcagg gagatggggg aactggctct gtgcttcatt
ccctgaaact gcatcacttt catggtagag atagatttgt
tcccatattg catgacatgg ctggtgttgg tgt

### Example 2.5

### Immunohistochemistry and Histology

Four μm thick sections of paraffin-embedded tissue were deparaffinized and rehydrated. Masson's trichrome staining was performed according to the manufacturer's instructions (American MasterTech Scientific, Lodi, Calif.). For immunohistochemistry, tissue sections were subjected to heat-mediated antigen retrieval treatment (Target Retrieval Solution, DAKO, Carpinteria, Calif.; DECLOAKING CHAMBER™, Biocare Medical, Concord, Calif.) according to the manufacturer's instructions, followed by blocking of nonspecific background staining (Serum-Free Protein Block, DAKO, Carpinteria, Calif.). Immunofluorescent detection of legumain was achieved using a sheep anti-mouse legumain primary antibody (R&D Systems, Minneapolis, Minn.) and a donkey anti-sheep Alexa594 secondary antibody or a donkey anti-sheep Alexa488 secondary antibody (Molecular Probes, Eugene, Oreg.). Chromogenic detection of legumain was performed using a sheep anti-mouse legumain primary antibody (R&D Systems, Minneapolis, Minn.) and a rabbit anti-sheep IgG conjugated to alkaline phosphatase secondary antibody. Detection of the signal was obtained using 5-bromo-4-chloro-3-indolyl phosphate/nitroblue tetrazolium substrate (BCIP/NBT, Vector Laboratories, Burlingame, Calif.) with added levamisole solution (Vector Laboratories) and nuclear Fast Red counterstaining. Immunodetection of CD68 was obtained using a rat anti-mouse CD68 primary antibody (Serotec, Raleigh, N.C.) and a rabbit anti-rat Alexa488 secondary antibody (Molecular Probes, Eugene, Oreg.). For double immunofluorescent staining of CD68 and legumain, a cocktail of CD68 and legumain primary antibodies was used before appropriate secondary antibodies were applied as described above.

Immunodetection of P-selectin was obtained using a goat anti-mouse P-selectin primary antibody conjugated to biotin (R&D Systems, Minneapolis, Minn.) that was applied to tissue sections in which endogenous biotin and avidin had been blocked using a commercial kit (#X0590, DAKO, Carpinteria, Calif.). The immunofluorescent signal was then detected using streptavidin conjugated to Alexafluor488 (S32354, Molecular Probes, Eugene, Oreg.) in slides that were mounted in VECTASHIELD® Hardset Mounting Medium with DAPI (Vector Laboratories, Burlingame, Calif.). For double immunofluorescent staining of P-selectin and legumain, a cocktail of the P-selectin primary antibody described above and a rat anti-mouse legumain (R&D Systems, Cat# MAB2058, Clone 301417, Minneapolis, Minn.) primary antibody was used. Detection of P-selectin was obtained as described herein and the secondary antibody used to detect legumain in that case was a rabbit anti-rat antibody conjugated to Alexafluor594.

### Example 2.6

### Results

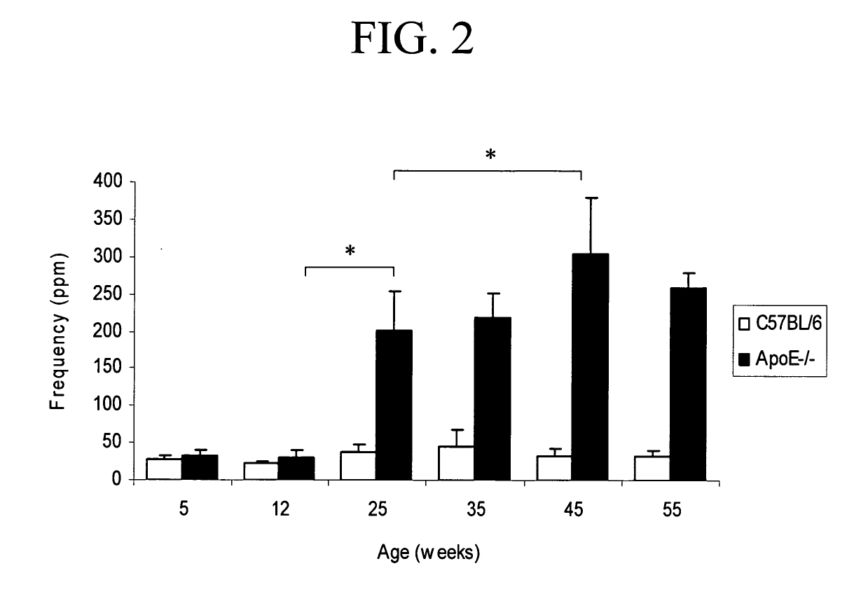

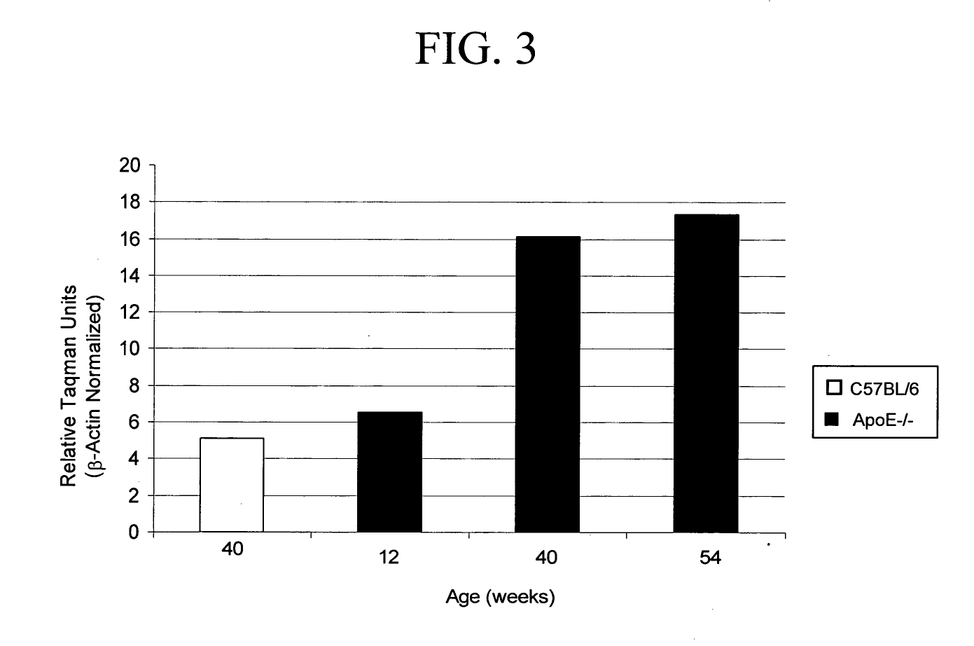

Legumain mRNA was identified as one of the genes upregulated in the aortic arch of ApoE−/− mice beginning at 25 weeks of age (statistical significance is denoted by asterisks), but was expressed at a low level that remained unchanged over time in the C57BL/6 (wild type (WT)) control animals (FIG. 2). TAQMAN® real-time PCR analysis confirmed the increase in legumain mRNA in adult ApoE−/− mice (at 40 and 54 weeks) compared to either 12-week old ApoE−/− mice or control 40-week old C57BL/6 (WT) control mice (FIG. 3). In situ hybridization detected legumain mRNA expressed in 55-week old ApoE−/− aortic arch, but not in a 45-week old C57BL/6 control section or sections stained with control probes (data not shown).

Immunohistochemical staining demonstrated that legumain protein was expressed in lesions at the aortic sinus in ApoE−/− mice (data not shown). In the aortic sinus, legumain expression was first detectable in 2-month old ApoE−/− mice (data not shown). Increased expression was detected in older mice in the developing atherosclerotic plaques. In 1-year old ApoE−/− mice, legumain was found within the atherosclerotic lesions in the areas of infiltrated inflammatory cells (data not shown). Legumain was not detected in the aortic sinus of adult C57BL/6 control mice (data not shown).

### Example 3

### Legumain is Expressed in the ApoE−/− Mouse Model of Accelerated Atherosclerosis

To determine whether legumain expression was limited to atherosclerotic lesions developing spontaneously in ApoE−/− mice, the expression pattern of legumain was analyzed in a model of accelerated atherosclerosis following vascular injury.

### Example 3.1

### Preparation of a Mouse Model of Accelerated Atherosclerosis

A subset of animals underwent left carotid artery ligation as previously described (A. Kumar and V. Lindner (1997) Arterioscler. Thromb. Vasc. Biol. 17:2238-44). Briefly, 8-10 week old ApoE KO mice were anesthetized with a solution of ketamine (100 mg/kg body wt) and xylazine (20 mg/kg) injected intraperitoneally. The left common carotid artery was exposed through a small midline incision in the neck. The artery was completely ligated just proximal to the carotid bifurcation to disrupt blood flow. The animals were allowed to recover for 4 weeks. At the end of the recovery period, animals were euthanized, perfused with saline and 4% paraformaldehyde as described herein and 5 mm-long segments of the left and right carotid arteries were collected to be embedded in paraffin blocks for analysis by immunohistochemistry.

### Example 3.2

### Results

Immunohistochemical staining demonstrated that legumain expression was detected in the neointimal lesions in the injured carotid arteries at four weeks after ligation (data not shown). Control staining using normal IgG did not detect any signals (data not shown). Staining of uninjured carotid artery with anti-legumain antibody did not reveal any signals (data not shown).

### Example 4

### Legumain is Expressed in Foam Cells of Atherosclerotic Lesions

To identify the type of cell associated with legumain in atherosclerotic lesions, cells were immunostained for both legumain and the macrophage marker CD68.

### Example 4.1

### Immunostaining of Atherosclerotic Lesions for Legumain and Macrophage Markers

Immunodetection of CD68 was obtained using a rat anti-mouse CD68 primary antibody (Serotec, Raleigh, N.C.) and a rabbit anti-rat Alexa488 secondary antibody (Molecular Probes, Eugene, Oreg.). For double immunofluorescent staining, a cocktail of CD68 and legumain primary antibodies was used before appropriate secondary antibodies were applied as described above. Chromogenic detection of legumain was performed as described herein.

### Example 4.2

### Results

Chromogenic and immunofluorescent staining revealed that legumain protein localized to atherosclerotic plaques within the coronary arteries (data not shown). Moreover, legumain colocalized with the macrophage marker CD68 in lesions developing at the aortic sinus, indicating that the major cell types expressing legumain in atherosclerotic plaques were macrophages and foam cells (data not shown).

### Example 5

### Legumain is Expressed by Arterial Endothelial Cells of Aortic Sinus

To determine whether legumain is expressed by arterial endothelial cells, cells were immunostained for both legumain and the endothelial marker, P-selectin.

### Example 5.1

### Immunostaining of Atherosclerotic Lesions for Legumain and Endothelial Cell Markers

Immunodetection of P-selectin was performed using a goat anti-mouse P-selectin primary antibody conjugated to biotin (R&D Systems), followed by streptavidin conjugated to Alexafluor488 (Molecular Probes, Eugene, Oreg.). For double immunofluorescent staining, a cocktail of legumain and P-selectin primary antibodies was used before the secondary antibody and streptavidin were applied.

### Example 5.2

### Results

Immunofluorescent staining revealed that legumain was expressed by arterial endothelial cells of the aortic sinus of ApoE−/− mice aged 2 months to 1 year, including endothelial cells overlaying early inflammatory lesions (data not shown).

### Example 6

### Legumain is Expressed within Kidney Proximal Tubule Cells and Endothelial Cells of the Renal Arteries of ApoE−/− Mice

Due to the involvement of vascular dysfunction and inflammatory processes in renal pathologies, the expression of legumain and was measured in kidneys of ApoE KO and C57BL/6 mice.

### Example 6.1

### Materials

Kidneys from male ApoE KO and C57BL/6 mice were harvested, prepared, and subjected to immunohistochemistry as described above in order to assess the expression of legumain in kidneys and renal arteries. P-selectin was used as a marker for endothelial cells.

### Example 6.2

### Results

Immunodetection of legumain protein in kidneys isolated from ApoE KO (ApoE−/−) mice revealed that legumain was predominantly expressed in kidney proximal tubule cells (data not shown). Double immunostaining for legumain and p-selectin in C57BL/6 mice revealed that the two proteins colocalize and that legumain is consistently expressed by endothelial cells in mouse renal arteries (data not shown).

### Example 7

### Expression of Legumain in Human Atherosclerotic Lesions

To determine whether legumain was also expressed in human atherosclerotic lesions, human coronary artery sections were immunostained for legumain expression.

### Example 7.1

### Human Coronary Artery Immunostaining

Human coronary arteries from a 57 year old female with advanced atherosclerotic plaques were stained for legumain using a goat anti-human legumain primary antibody (R&D Systems) and a biotinylated rabbit anti-goat IgG secondary antibody.

### Example 7.2

### Results

Immunohistochemistry experiments indicated that legumain protein was not expressed in normal human coronary arteries. In contrast, legumain protein was mainly expressed by inflammatory cells in advanced coronary atherosclerotic plaques, in regions of foam cell overlaying fibrotic and calcified portions of the plaques, as well as in sites of neovascularization (data not shown).

### Example 8

### Legumain Expression and Activity Increases in Differentiated THP1 Monocytes and Activated Primary Human Macrophages

The expression of legumain and the enzymatic activity of legumain were measured in differentiated THP-1 macrophages and CSF-stimulated human macrophages.

### Example 8.1

### Cell Culture

Human monocytic THP-1 cells were obtained from ATCC and maintained in RPMI 1640 medium (ATCC) containing 10% fetal bovine serum and β-mercaptoethanol. THP-1 monocytes were differentiated into macrophages over three days in the growth medium containing 100 μg/ml phorbol 12-myristate 13-acetate (PMA) (Sigma).

### Example 8.2

### Human Primary Cell Culture Conditions

Human monocytes were isolated from the buffy coat byproduct of a volunteer blood donor using Rosettesep Human Monocyte Enrichment cocktail (StemCell Technologies, Vancouver, BC) following the manufacturer's protocol. Monocyte purity was determined to be 88% by Celldyne clinical cell counter (Abbott, Alameda, Calif.). Monocytes were suspended to 2×10⁶ cells/ml in RPMI supplemented with penicillin, streptomycin, L-glutamine, 2.5 mM Hepes (Sigma), and 10% heat-inactivated fetal bovine serum (Hyclone, Logan, Utah) and further enriched by a 2 hour adherence to plastic in 10 cm tissue culture dishes (Falcon, BD Biosciences, Rockville, Md.) at 37° C.

Adherent cells were washed vigorously and cultured for 72 hours in RPMI containing 0.25% FBS in the presence or absence of 20 ng/ml recombinant human macrophage colony stimulating factor (M-CSF) (R&D Systems, Minneapolis, Minn.). Supernatant was harvested, clarified by centrifugation, and stored at −80° C. Cells were scraped into 0.5 ml modified RIPA (50 mM Tris, 150 mM NaCl, 1 mM EDTA, 1% NP40, 0.25% deoxycholic acid) containing Complete Mini protease inhibitor (Roche, Nutley, N.J.) and incubated 15 minutes on ice. Insoluble material was removed by centrifugation and the supernatants were stored at −80° C.

### Example 8.3

### Western Blot Analysis

Protein extracts were prepared from THP-1 cells or human macrophages by lysing cells in lysis buffer (50 mM Tris-HCl, pH 7.5, 150 mM NaCl, 5 mM EDTA, 1% Triton-X, 5 mM DTT) supplemented with protease inhibitor tablet (Roche Diagnostics, Nutley, N.J.). Cell lysates were subsequently cleared by centrifugation. The supernatant was collected and resolved on SDS-PAGE gel. The proteins were transferred to PVDF membranes (Bio-Rad, Hercules, Calif.) and incubated with primary and secondary antibodies before ECL detection (Roche Diagnostics, Nutley, N.J.). Antibodies used include an anti-actin polyclonal antibody (Santa Cruz Biotechnology, Santa Cruz, Calif.), and an anti-legumain polyclonal antibody (R&D Systems, Minneapolis, Minn.).

### Example 8.4

### Legumain Activity Assay

A fluorimetric assay measuring legumain protease activity was performed as previously described with some modifications (Johansen et al. (1999) Anal. Biochem. 273: 278-83). Cell extract (20-50 μl) was added to each well of 96-well plate, to which 150 μl of assay buffer containing 10 nM of the substrate Z-AAN-MCA (Peptide Institute) was added. The plate was incubated at room temperature for 5 min and measured for fluorescence in a VICTOR 3™ fluorescence plate reader (PerkinElmer, Wellesley, Mass.) using an excitation filter of 360 nm and an emission filter of 460 nm. Repeated measurements were carried out once every 5 min over a period of 20 min at room temperature. The increase of fluorescence over time was plotted.

### Example 8.5

### TAQMAN™ Real-time Quantitative PCR

RNA was isolated and purified from mouse tissues or THP-1 cells using RNEASY™ kit (Qiagen, Valencia, Calif.) according to the manufacturer's instructions. Using an ABI PRISM™ 7000 Sequence Detection System (PE Applied Biosystems, Foster City, Calif.), legumain mRNA levels were measured by TAQMAN™ real-time quantitative PCR as previously described (Lake et al. (2005) J. Lipid Res. 46:2477-87) with custom-made TAQMAN™ reagents (PE Applied Biosystems, Foster City, Calif. or Eurogentec, San Diego, Calif.). The following primers were used: forward primer (SEQ ID NO:14) and reverse primer (SEQ ID NO:15). Data were analyzed according to manufacturer's instructions.

### Example 8.6

### Results

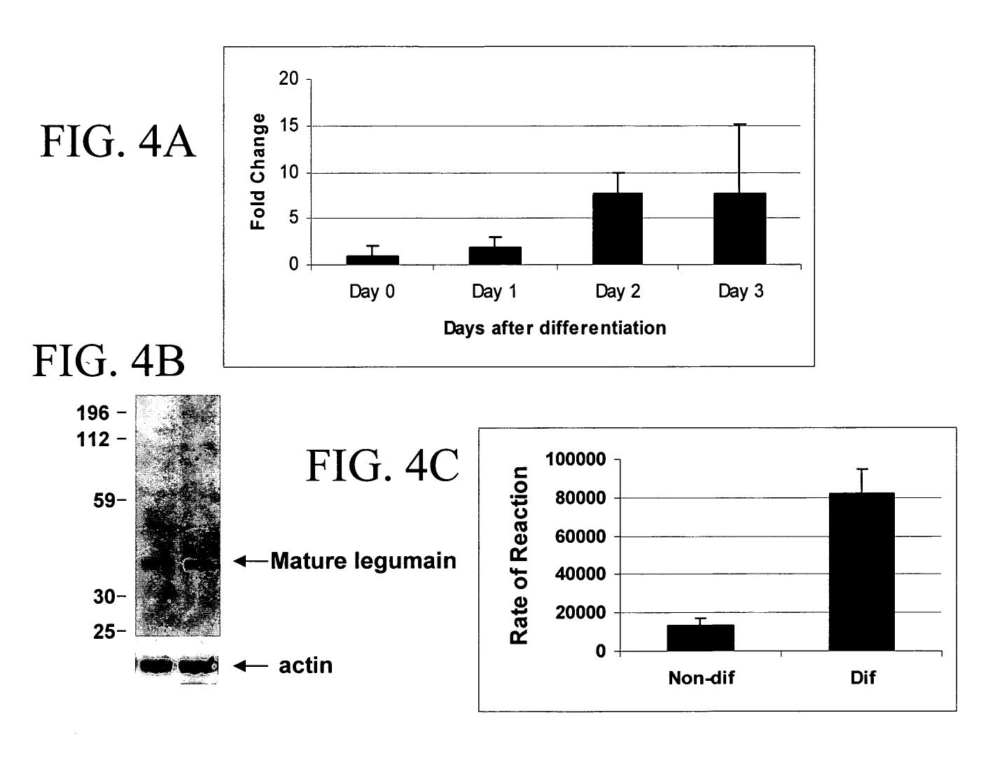

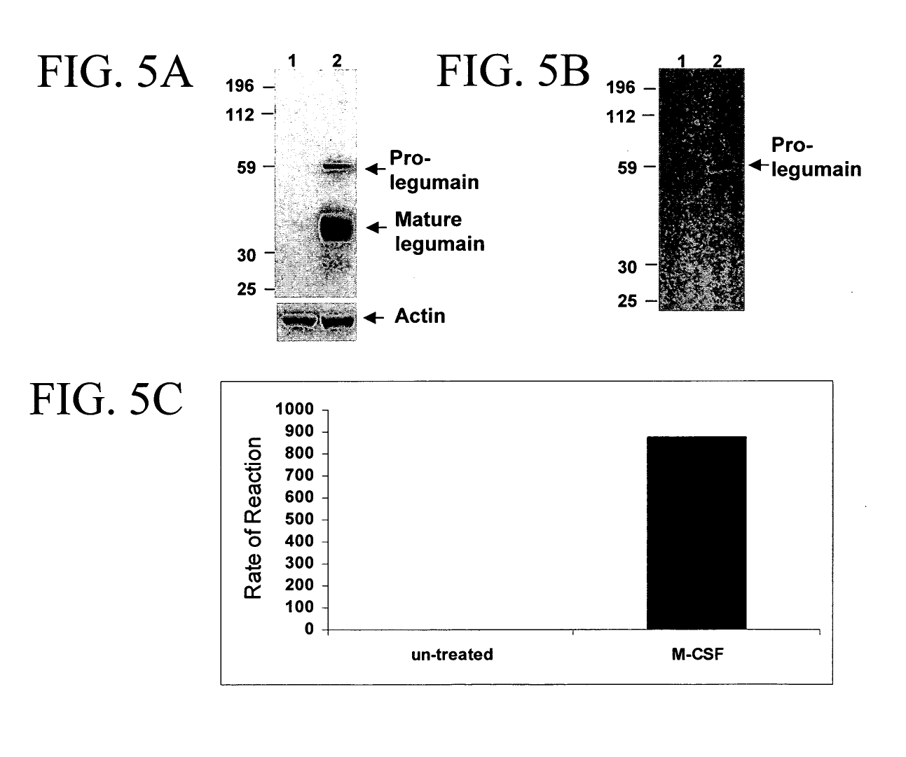

Compared with undifferentiated THP-1 monocytes, legumain mRNA (FIG. 4A), protein (FIG. 4B), and protease activity (FIG. 4C) were markedly increased in the differentiated THP-1 macrophages. In addition, human macrophages differentiated with M-CSF also exhibited increased legumain protein (FIG. 5A) and activity (FIG. 5C). By Western analysis, secreted legumain (as pro-legumain) was detected in the conditioned media of M-CSF-treated human macrophages (FIG. 5B). These results demonstrate that differentiated macrophages express high levels of legumain and are capable of secreting legumain into the extracellular environment.

### Example 9

### Legumain Chemoattractive Properties Towards Differentiated Human Monocytes In Vitro

To determine whether the legumain released into the extracellular environment acts as a chemoattractant molecule that contributes to monocyte recruitment, migration of primary human monocytes towards the purified proform of legumain was tested in modified Boyden chambers.

### Example 9.1

### Human Monocytes Migration Assay

Human monocytes were isolated from the blood of healthy donors using the negative selection method with the Monocyte Isolation Kit (Miltenyi Biotech) according to manufacturer's instructions. The purity of isolated monocytes was confirmed by flow cytometry on CD14-stained cells. Cells were tested in a modified Boyden chamber assay using Multi Screen 96-well filtration plate (Millipore, 5.0 μm pore size). Serum-starved monocytes were added to the top chamber (20,000 cells/well), and serum-free culture medium was added to the bottom chamber with or without purified legumain (R&D Systems). VEGF (R&D Systems) was used as a positive control. Cells that migrated into the bottom chamber were quantified using a luminescent cell viability assay (CellTiter-Glo® Assay, Promega, Madison, Wis.) at 2 hours.

### Example 9.2

### Results

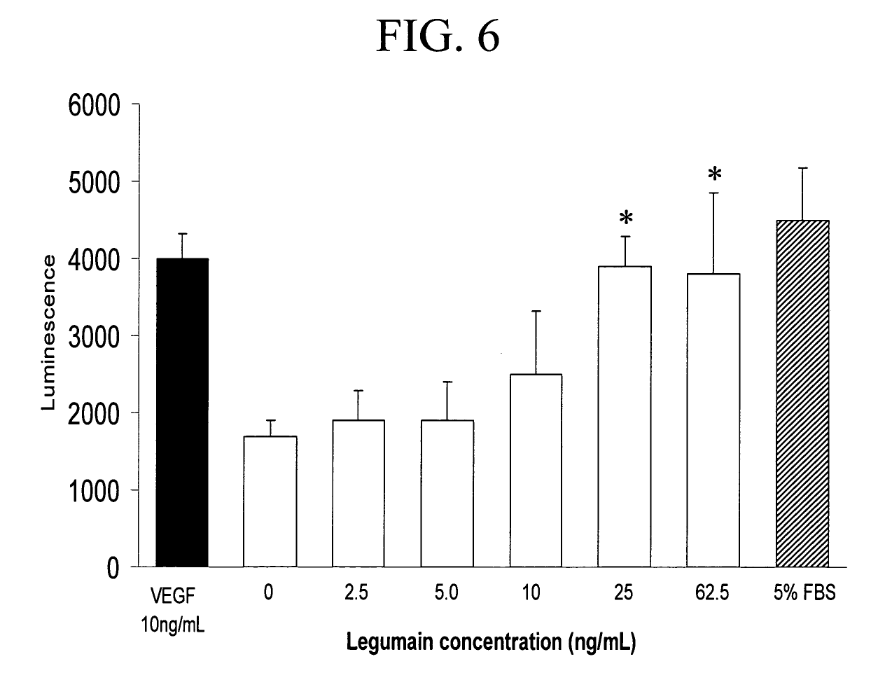

A dose-dependent increase in the migration of human monocytes towards legumain was observed (FIG. 6). A dose of 25 ng/mL was found to be the minimal effective concentration of legumain, inducing a 2.3 fold increase in migration of monocytes relative to control. The number of migrated cells in response to legumain at 25 ng/mL and VEGF at 10 ng/mL was similar.

### Example 10

### Active Legumain is Expressed on the Cell Surface of Recombinant Overexpressing Cells

In order to determine if legumain is expressed on cell surfaces, CHO cells overexpressing mouse legumain were generated and assayed for cell surface legumain. To determine whether such cell surface legumain is enzymatically active, HEK293 cells overexpressing legumain were assayed for legumain protease activity.

### Example 10.1

### Immunofluorescence Staining of CHO Cells

CHO cells were infected with adenovirus expressing mouse legumain at MOI of 500. Forty-eight hours post infection, cells were fixed with 2% paraformaldehyde in PBS at 4° C. for 20 minutes. After blocking cells with blocking buffer (10% FBS, 3% BSA in PBS) at room temperature for 30 minutes, the cells were incubated with sheep anti-mouse legumain antibody (R&D Systems, Minneapolis, Minn.) or control normal sheep IgG (Santa Cruz Biotechnology, Santa Cruz, Calif.) at room temperature for 1 hour. The cells were washed three times with PBS and incubated with Alexa488-conjugated donkey anti-sheep antibody (Molecular Probes, Eugene, Oreg.) at room temperature for 1 hour. After three washes with PBS, the cells were counterstained with Hoechst dye (Molecular Probes, Eugene, Oreg.) for 5 min at room temperature. The cells were subsequently photographed under fluorescent microscope at 40× magnification.

### Example 10.2

### HEK293 Cell-Surface Legumain Activity Assay

HEK293 cells were plated in 96-well black tissue culture plate (BD Biosciences) and infected with adenovirus expressing mouse legumain at MOI of 10. 24 hours post infection, the cells were washed once with legumain assay buffer [39.5 mM citric acid, 125 mM Na₂HPO₄ (pH 5.8), 1 mM EDTA, 0.8% NaCl], and 50 μl of assay buffer containing 10 nM of the substrate Z-AAN-MCA (Peptide Institute, Louisville, Ky.) was added to each well. The protease inhibitors cystatin C(R&D Systems, Minneapolis, Minn.) or E64 (Sigma) were included in the assay buffer at a concentration of 100 nM. The plates were incubated at 37° C. for 10 min and measured for fluorescence in a VICTOR 3™ fluorescence plate reader (PerkinElmer, Wellesley, Mass.) using an excitation filter of 360 nm and an emission filter of 460 nm. Repeated measurements were carried out once every 5 min over a period of 20 min at room temperature. The increase of fluorescence over time was plotted.

### Example 10.3

### Results

By immunofluorescent staining, legumain expression was detected on the surface of CHO cells infected with adenovirus expressing mouse legumain (data not shown). Cells stained with legumain antibody were positive for the protein, whereas the control normal sheep IgG stained cells only displayed background staining (data not shown).

By using nonpermeable assay conditions, the cell surface legumain activity in HEK293 cells infected with adenovirus expressing mouse legumain was directly measured on live HEK293 cells overexpressing mouse legumain. As shown in FIG. 7, the measured activity could be inhibited with cystatin C but not E64, indicating the activity was specific for legumain. Mock-infected HEK293 cells did not exhibit measurable activity in this assay (data not shown). Thus, cells are capable of expressing legumain on their surfaces in an enzymatically active form.

### Example 11

### Legumain-Mediated Endothelial Cell Migration and Proliferation

In order to determine if legumain is involved in cell migration and proliferation, e.g., endothelial cell migration as occurs during atherogenesis, the influence of legumain on wound healing was studied in HEK293 and HUVEC cultures.

### Example 11.1

### In Vitro Wound-Healing Assay

HEK293 cells (ATCC, Manassas, Va.) used at passage 10 and HUVECs (Cambrex, Walkersville, Md.) used at passage 3 were seeded onto single-chamber slides (Lab-Tek cat# 177410, Campbell, Calif.). HEK293 cells were cultured to >80% confluence in DMEM supplemented with 10% FBS and 1% penicillin/streptomycin/L-glutamine (Cat# 10378-016, Gibco, Invitrogen, Carlsbad, Calif.), and HUVECs were grown to >90% confluence in EGM (Cat# CC-3124, Cambrex, East Rutherford, N.J.). After washing the cells in serum-free medium, the monolayers were mechanically wounded using a cell scraper (Cat# 3010, Costar [Corning], Fisher Scientific, Pittsburgh, Pa.) to obtain a rectangular denuded area of 2.0×1.7 cm². Wounded cells were then incubated in serum-free medium supplemented with 0.05% delipidized BSA (BD Biosciences, Bedford, Mass.) and with VEGF (10 ng/mL, R&D Systems, Minneapolis, Minn.) or legumain (10 ng/mL or 25 ng/mL, R&D Systems, Minneapolis, Minn.) for 24 hours (HEK293 cells) or 20 hours (HUVECs). Wound healing was quantified by measuring the number of cells present in the area of the initial wound using a luminescent cell viability assay (CellTiter-Glo® Assay, Promega, Madison, Wis.).

### Example 11.2

### Results

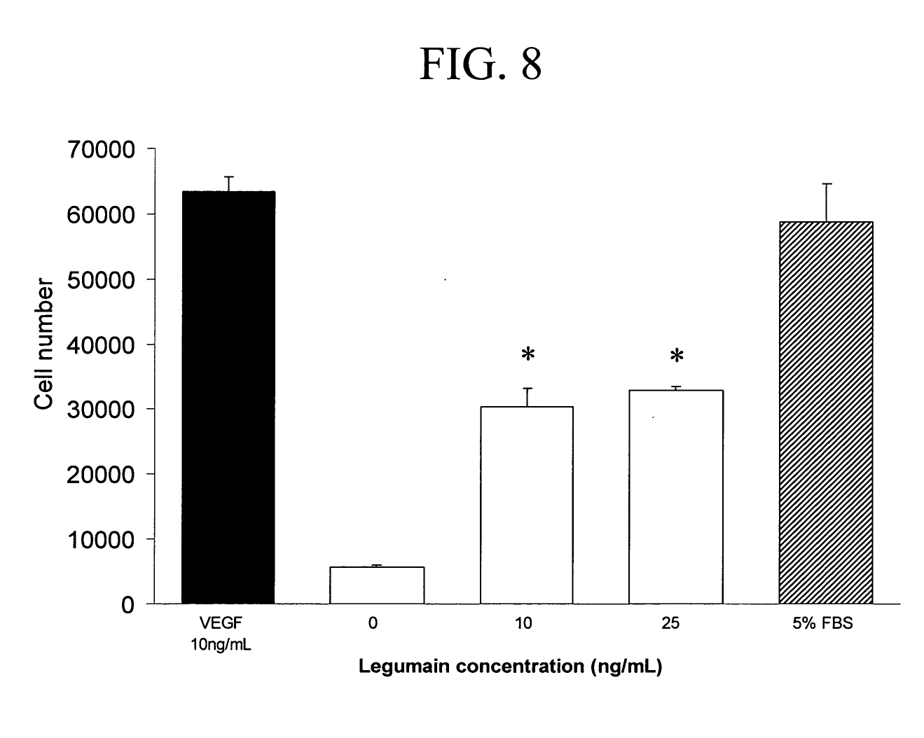

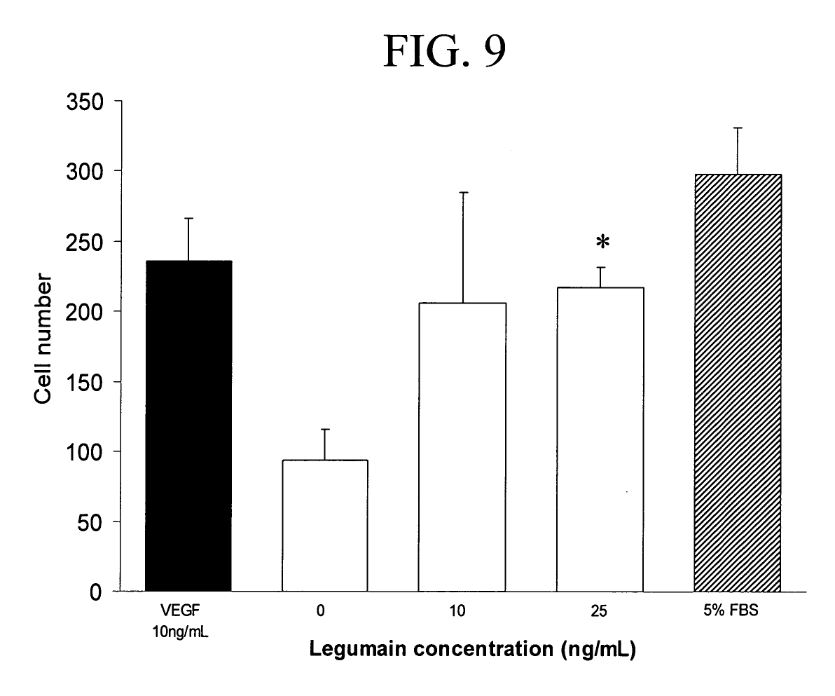

The data presented in FIGS. 8 and 9 reveal an increase in cell migration in response to stimulation with legumain at 10 ng/mL and 25 ng/mL relative to control (5% FBS and VEGF). Thus, legumain appears to be involved in endothelial cell migration; such migration occurs during atherogenesis, and may be involved in angiogenesis. Methods, therapeutics, etc. designed to diagnose, prognose, monitor, treat, ameliorate and/or prevent disorders and/or conditions involving angiogenesis (including, but not limited to, cancer and inflammatory disorders) are contemplated in the present invention.

### Example 12

### Legumain-Mediated Invasion of Endothelial Cells

In order to determine if legumain is also involved in endothelial cell invasion, e.g., as related to angiogenesis, HUVECs were tested in a modified Boyden chamber assay.

### Example 12.1

### Modified Boyden Chamber Assay

In a modified Boyden chamber assay, 25,000 serum starved HUVECs loaded into the top chamber were allowed to invade a Matrigel coated PET membrane (3.0 μm pore size, Becton Dickinson, BioCoat Angiogenesis System: Endothelial Cell Invasion) in response to purified legumain added to the bottom chamber. Cells that invaded through the Matrigel matrix were quantified using a luminescent cell viability assay (CellTiter-Glo® Assay, Promega, Madison, Wis.). VEGF was used a positive control.

### Example 12.2

### Results

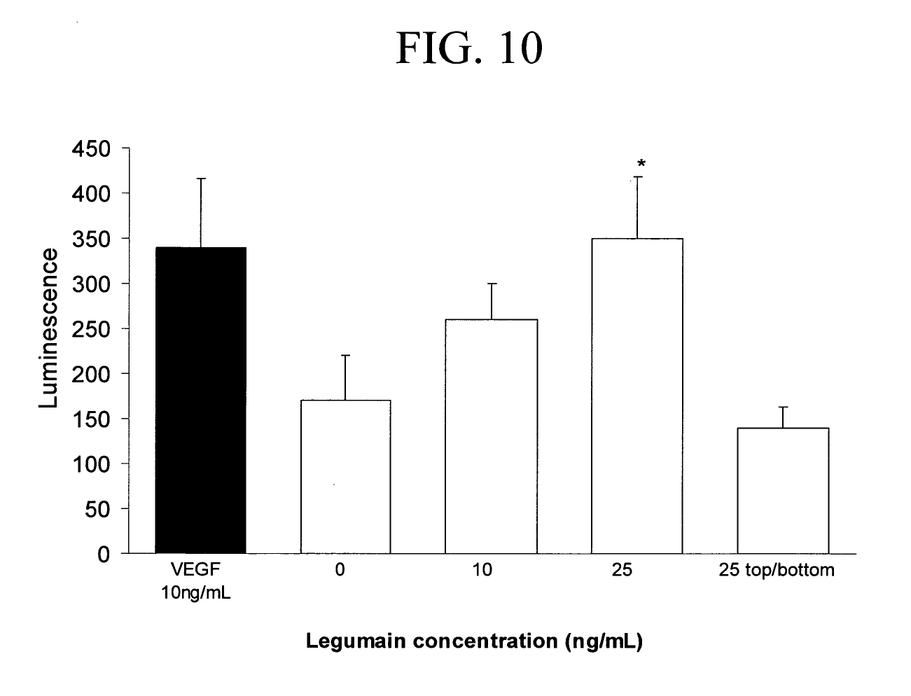

When HUVECs were tested for their invasive properties in a modified Boyden chamber assay, a dose-dependent increase in the number of cells invading a Matrigel coated membrane was observed in response to legumain, with 25 ng/mL legumain inducing a response similar to 10 ng/mL VEGF during a 22 hour period (FIG. 10).

### Example 13

### Legumain is Highly Expressed in Diseased Paws of the Collagen-Induced Arthritis (CIA) Mouse Model of Arthritis

To determine if legumain plays a role in other disorders marked by increased macrophage and monocyte activity, e.g., tuberculosis and rheumatoid arthritis, the expression of legumain in the Collagen-Induced Arthritis (CIA) model of arthritis was examined.

### Example 13.1

### Collagen-Induced Arthritis (CIA) Model

Male DBA/1 mice were obtained from Jackson Laboratories, Bar Harbor, Me. Arthritis was induced using bovine collagen type II (Chondrex, Redmond, Wash.) dissolved in 0.1 M acetic acid and emulsified in an equal volume of Complete Freund's Adjuvant (Sigma) containing 1 mg/ml Mycobacterium tuberculosis (strain H37RA). Mice were injected subcutaneously with a 100 μg of the collagen mixture in the base of the tail. On day 21 mice received an additional subcutaneous injection in the base of the tail with 100 μg of bovine collagen II in 0.1M acetic acid mixed with an equal volume of Incomplete Freund's Adjuvant (Sigma). Naïve animals received no collagen. Mice were monitored at least three times a week for disease severity. Limbs were assigned a clinical score based on the index:0=normal; P=prearthritic characterized by focal erythema on the tips of digits; 1=visible erythema accompanied by 1-2 swollen digits; 2=pronounced erythema, paw swelling and/or multidigit swelling; 3=massive swelling extending into ankle or wrist joint; 4=difficulty in using limb or joint rigidity; resulting in a maximum total body score of 16. At various intervals post onset of disease animals were sacrificed, tissues were harvested, paws were fixed in 4% paraformaldehyde pH 7.47, decalcified in 20% EDTA (pH 8.0) and embedded in paraffin for in situ hybridization.

### Example 13.2

### Results

Legumain signal was strongly positive in diseased paws (clinical score 3) in CIA mice, but absent in normal control paws, indicating that legumain expression was upregulated with disease in this arthritis model (data not shown). These results suggest the involvement of legumain in inflammatory disorders in which macrophages and monocytes are chronically involved.

### Example 14

### Identification and Characterization of the Legumain Splice Variant ZB-1

To determine if additional legumain proteins or transcripts may contribute to vascular and inflammatory disorders, cDNAs from human adrenal glands were screened to identify proteins with high homology to human legumain.

### Example 14.1

### Isolation of ZB-1 from Human Adrenal Gland

cDNAs from human adrenal gland were subcloned into the Adori expression vector. With the Adori vector, expression is controlled by the cytomegalovirus (CMV) immediate early promoter and enhancer. Ad5 E1a deleted recombinant adenovirus was generated by homologous recombination in a human embryonic kidney cell line 293 (HEK293, ATCC, Manassas, Va.). Recombinant adenovirus was isolated and subsequently amplified on 293 cells. The virus was released from infected 293 cells by three cycles of freeze thawing. The virus was further purified by two cesium chloride centrifugation gradients and dialyzed against phosphate buffered saline (PBS) pH 7.2 at 4° C. Following dialysis, glycerol was added to a concentration of 10% and the virus was stored at −80° C. until use. The virus was characterized by assessing the following parameters; expression of the transgene, plaque forming units on 293 cells, particles/ml, endotoxin measurements, PCR analysis of the virus and sequence analysis of the legumain or ZB-1 coding region in the virus. Adenoviruses were tested for expression of recombinant protein by radiolabeling virus infected HEK293 cells and monitoring protein expression.

In order to monitor protein synthesis, cells were labeled for 6 hours with ³⁵S-methionine and ³⁵S-cysteine. HEK293 cells were plated in P60 culture plates at a density of 7.5×10⁵ cells/plate in 4 ml of complete medium (Dulbecco's Modified Eagle's Media (DME)+10% heat inactivated fetal bovine serum (FBS)+Penicillin/Streptomycin (Penn/Strep)+Glutamine at 2 mM). Twenty-four hours later the media was replaced with 2 ml of reduced serum medium (DME+2% FBS+Penn/Strep+Glutamine) containing adenovirus at an MOI of 20-100. Plates were incubated for 2 hours and then fed 3 ml of complete media and incubated for an additional 24 hours. The following day medium was removed and replaced with 2 mls of serum free-, methionine/cysteine free-DME. Cells were incubated for one hour, then the medium was removed and replaced with 1 ml of serum free-DME supplemented with ³⁵S-methionine and ³⁵S-cysteine. Cells were incubated for 15 minutes and 1 ml of DME containing methionine and cysteine+2% FBS+aprotonin (1:100 aprotonin; Sigma-6279) was added. Cells were incubated for an additional 4 hours, after which the 2 ml of medium was collected and centrifuged at a low speed to remove cells that may have detached during labeling. The cleared medium was transferred to a clean tube containing soybean trypsin inhibitor (1 mg/ml) and 20 μl phenylmethylsulphonylfluoride (1 mM). Radiolabeled conditioned media was stored at −2° C. for later analysis by SDS polyacrylamide gel electrophoresis and autoradiography.

### Example 14.2

### Results

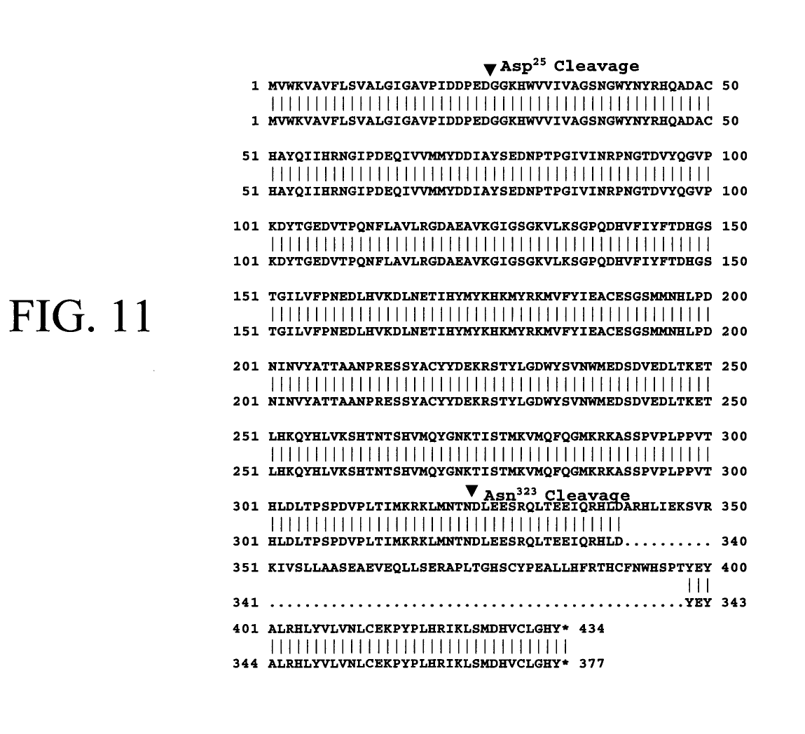

As shown in FIG. 11, ZB-1 encodes a novel secreted protein expressed in human adrenal gland. ZB-1 appears to be a splice variant of human legumain, with 100% identity to human legumain, except for the 57 amino acid residues deleted (equivalent to amino acids 341-397 of legumain). ZB-1 is a secreted protein that was found in the medium of HEK293 cell cultures infected with adenovirus overexpressing ZB-1 (data not shown).

What is claimed is:

1. A polynucleotide comprising the nucleic acid sequence set forth in SEQ ID NO:11.

2. A polypeptide comprising the amino acid sequence set forth in SEQ ID NO:12, amino acids 21 to 323 of SEQ ID NO:12, or amino acids 25 to 323 of SEQ ID NO:12.

3. An antibody or antigen binding fragment thereof that specifically binds a mammalian ZB-1 polypeptide or a fragment of a mammalian ZB-1 polypeptide.

4. The antibody or antigen binding fragment thereof as in claim 3, wherein the mammalian ZB-1 polypeptide or the fragment of a mammalian ZB-1 polypeptide is derived from a human.

5. Use of a legumain antagonist and/or a ZB-1 antagonist for the preparation of a pharmaceutical composition for use in a method of treating, ameliorating, or preventing a vascular disorder or an inflammatory disorder, wherein the pharmaceutical composition comprises a therapeutically effective amount of the legumain antagonist and/or the ZB-1 antagonist, and a pharmaceutically acceptable carrier.

6. The use of a legumain antagonist and/or a ZB-1 antagonist of claim 5, wherein the legumain antagonist and/or ZB-1 antagonist is selected from the group consisting of inhibitory polynucleotides, inhibitory polypeptides, small molecules, antagonistic antibodies and antigen binding fragments thereof.

7. A method for treating, ameliorating, or preventing a vascular disorder or an inflammatory disorder in a mammal comprising administering to the mammal a therapeutically effective amount of a legumain antagonist and/or a ZB-1 antagonist.

8. The method of claim 7, wherein the legumain antagonist and/or ZB-1 antagonist is selected from the group consisting of inhibitory polynucleotides, inhibitory polypeptides, small molecules, antagonistic antibodies, and antigen binding fragments thereof.

9. A method for treating, ameliorating, or preventing a vascular disorder or an inflammatory disorder in a mammal comprising contacting a cell or cell population of the mammal with a therapeutically effective amount of a legumain antagonist and/or a ZB-1 antagonist.

10. The method claim 9, wherein the cell or cell population comprises a macrophage, a monocyte, a vascular endothelial cell, a foam cell, or a mixture of monocytes, macrophages, vascular endothelial cells and/or foam cells.

11. The method of claim 10, wherein the cell or cell population secretes legumain and/or ZB-1.

12. A method for decreasing the level of legumain and/or ZB-1 activity, expression, and/or secretion in a mammal comprising administering to the mammal a legumain antagonist and/or a ZB-1 antagonist in an amount sufficient to decrease the level of activity, expression, and/or secretion of legumain and/or ZB-1 in the mammal.

13. A method for monitoring the course of a treatment for a vascular disorder or inflammatory disorder in a patient, comprising:
(a) measuring the level of activity, expression and/or secretion of legumain and/or ZB-1 in a cell or cell population from the patient;
(b) administering a legumain antagonist and/or a ZB-1 antagonist to the patient; and
(c) measuring the level of activity, expression and/or secretion of legumain and/or ZB-1 in a cell or cell population from the patient following administration of the legumain antagonist and/or ZB-1 antagonist,
wherein a lower level of activity, expression and/or secretion of legumain and/or ZB-1 in the cell or cell population from the patient following administration of the legumain antagonist and/or ZB-1 antagonist, in comparison to the level of activity, expression and/or secretion of legumain and/or ZB-1 in the cell or cell population from the patient prior to administration of the legumain antagonist and/or ZB-1 antagonist, provides a positive indication of the effect of the treatment for the vascular disorder or inflammatory disorder in the patient.

14. A method for inhibiting cell migration in a mammal comprising administering to the mammal a legumain antagonist and/or a ZB-1 antagonist.

15. The method of claim 14, wherein the legumain antagonist and/or ZB-1 antagonist is selected from the group consisting of inhibitory polynucleotides, inhibitory polypeptides, small molecules, antagonistic antibodies, and antigen binding fragments thereof.

16. A method for promoting wound healing in a mammal comprising administering to the mammal a legumain agonist and/or a ZB-1 agonist.

17. A method for inhibiting angiogenesis in a mammal comprising administering to the mammal a legumain antagonist and/or a ZB-1 antagonist.

18. The method of claim 17, wherein the legumain antagonist and/or ZB-1 antagonist is selected from the group consisting of inhibitory polynucleotides, inhibitory polypeptides, small molecules, antagonistic antibodies, and antigen binding fragments thereof.

19. A method for inhibiting proliferation of endothelial cells in a mammal comprising administering to the mammal a legumain antagonist and/or a ZB-1 antagonist.

20. A method for inhibiting tumor metastasis in a mammal comprising administering to the mammal a legumain antagonist and/or ZB-1 antagonist.
